(19) United States
(12) Patent Application Publication
(10) Pub. No.: US 2013/0060019 A1
(43) Pub. Date: Mar. 7, 2013

(54) Compounds for Use in Treating Neurodegenerative Disorders, Synthesis Thereof, and Intermediates Thereto

(71) Applicants: Brian Scott Bronk, East Lyme, CT (US); Wesley Francis Austin, Cambridge, MA (US); Steffen Phillip Creaser, Cambridge, MA (US); Nathan Oliver Fuller, Somerville, MA (US); Jed Lee Hubbs, Cambridge, MA (US); Ruichao Shen, West Roxbury, MA (US)

(72) Inventors: Brian Scott Bronk, East Lyme, CT (US); Wesley Francis Austin, Cambridge, MA (US); Steffen Phillip Creaser, Cambridge, MA (US); Nathan Oliver Fuller, Somerville, MA (US); Jed Lee Hubbs, Cambridge, MA (US); Ruichao Shen, West Roxbury, MA (US)

(73) Assignee: SATORI PHARMACEUTICALS, INC., Cambridge, MA (US)

(21) Appl. No.: 13/605,961

(22) Filed: Sep. 6, 2012

Related U.S. Application Data
(60) Provisional application No. 61/532,051, filed on Sep. 7, 2011.

(51) Int. Cl. C07J 71/00

(52) U.S. Cl. 540/48

(57) ABSTRACT

The present invention provides methods of making a compound of formula II:

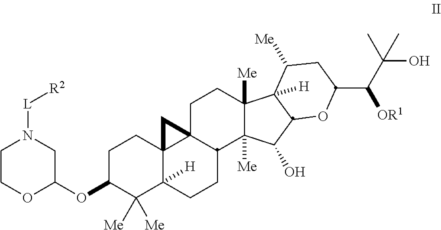

or a pharmaceutically acceptable salt thereof, wherein R¹, R², and L are as defined and described herein.

### RELATED APPLICATION

This application claims the benefit of U.S. Provisional Application Ser. No. 61/532,051, filed Sep. 7, 2011, the disclosure of which is incorporated in its entirety herein by reference.

### FIELD OF THE INVENTION

The present invention relates to methods for synthesizing compounds useful for treating neurodegenerative disorders, derivatives thereof, and to intermediates thereto.

### BACKGROUND OF THE INVENTION

The central role of the long form of amyloid beta-peptide, in particular Aβ(1-42), in Alzheimer's disease has been established through a variety of histopathological, genetic and biochemical studies. See Selkoe, D J, Physiol. Rev. 2001, 81:741-766, Alzheimer's disease: genes, proteins, and therapy, and Younkin S G, J. Physiol. Paris. 1998, 92:289-92, The role of A beta 42 in Alzheimer's disease. Specifically, it has been found that deposition in the brain of Aβ(1-42) is an early and invariant feature of all forms of Alzheimer's disease. In fact, this occurs before a diagnosis of Alzheimer's disease is possible and before the deposition of the shorter primary form of A-beta, Aβ(1-40). See Parvathy S, et al., Arch. Neurol. 2001, 58:2025-32, Correlation between Abetax-40-, Abetax-42-, and Abetax-43-containing amyloid plaques and cognitive decline. Further implication of Aβ(1-42) in disease etiology comes from the observation that mutations in presenilin (gamma secretase) genes associated with early onset familial forms of Alzheimer's disease uniformly result in increased levels of Aβ(1-42). See Ishii K., et al., Neurosci. Lett. 1997, 228:17-20, Increased A beta 42(43)-plaque deposition in early-onset familial Alzheimer's disease brains with the deletion of exon 9 and the missense point mutation (H163R) in the PS-1 gene. Additional mutations in the amyloid precursor protein APP raise total Aβ and in some cases raise Aβ(1-42) alone. See Kosaka T, et al., Neurology, 48:741-5, The beta APP717 Alzheimer mutation increases the percentage of plasma amyloid-beta protein ending at A beta42(43). Although the various APP mutations may influence the type, quantity, and location of Aβ deposited, it has been found that the predominant and initial species deposited in the brain parenchyma is long Aβ (Mann). See Mann D M, et al., Am. J. Pathol. 1996, 148:1257-66, “Predominant deposition of amyloid-beta 42(43) in plaques in cases of Alzheimer's disease and hereditary cerebral hemorrhage associated with mutations in the amyloid precursor protein gene”.

In early deposits of Aβ, when most deposited protein is in the form of amorphous or diffuse plaques, virtually all of the Aβ is of the long form. See Gravina S A, et al., J. Biol. Chem., 270:7013-6, Amyloid beta protein (A beta) in Alzheimer's disease brain. Biochemical and immunocytochemical analysis with antibodies specific for forms ending at A beta 40 or A beta 42(43); Iwatsubo T, et al., Am. J. Pathol. 1996, 149:1823-30, Full-length amyloid-beta (1-42(43)) and amino-terminally modified and truncated amyloid-beta 42(43) deposit in diffuse plaques; and Roher A E, et al., Proc. Natl. Acad. Sci. USA. 1993, 90:10836-40, beta-Amyloid-(1-42) is a major component of cerebrovascular amyloid deposits: implications for the pathology of Alzheimer disease. These initial deposits of Aβ(1-42) then are able to seed the further deposition of both long and short forms of Aβ. See Tamaoka A, et al., Biochem. Biophys. Res. Commun. 1994, 205:834-42, Biochemical evidence for the long-tail form (A beta 1-42/43) of amyloid beta protein as a seed molecule in cerebral deposits of Alzheimer's disease.

In transgenic animals expressing Aβ, deposits were associated with elevated levels of Aβ(1-42), and the pattern of deposition is similar to that seen in human disease with Aβ(1-42) being deposited early followed by deposition of Aβ(1-40). See Rockenstein E, et al., J. Neurosci. Res. 2001, 66:573-82, Early formation of mature amyloid-beta protein deposits in a mutant APP transgenic model depends on levels of Abeta(1-42); and Terai K, et al., Neuroscience 2001, 104:299-310, beta-Amyloid deposits in transgenic mice expressing human beta-amyloid precursor protein have the same characteristics as those in Alzheimer's disease. Similar patterns and timing of deposition are seen in Down's syndrome patients in which Aβ expression is elevated and deposition is accelerated. See Iwatsubo T., et al., Ann. Neurol. 1995, 37:294-9, Amyloid beta protein (A beta) deposition: A beta 42(43) precedes A beta 40 in Down syndrome.

Accordingly, selective lowering of Aβ(1-42) thus emerges as a disease-specific strategy for reducing the amyloid forming potential of all forms of Aβ, slowing or stopping the formation of new deposits of Aβ, inhibiting the formation of soluble toxic oligomers of Aβ, and thereby slowing or halting the progression of neurodegeneration.

### DETAILED DESCRIPTION OF CERTAIN EMBODIMENTS

As described herein, the present invention provides methods for preparing compounds useful as modulators of amyloid-beta production. Such compounds are useful for treating or lessening the severity of a neurodegenerative disorder. The present invention also provides intermediates useful in carrying out such synthetic methods.

In certain embodiments, the present invention provides methods for preparing a compound of Formula II depicted below:

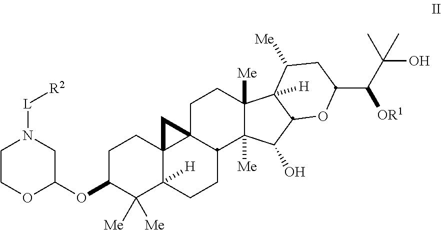

or a pharmaceutically acceptable salt thereof, wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R.

### DEFINITIONS

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-6 aliphatic carbon atoms. In yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Exemplary monocyclic hydrocarbons include, for example, cyclopropyl, cyclobutyl, cyclopentyl, and the like. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. In other embodiments, an aliphatic group may have two geminal hydrogen atoms replaced with oxo (a bivalent carbonyl oxygen atom ═O), or a ring-forming substituent, such as —O-(straight or branched alkylene or alkylene)-O— to form an acetal or ketal. The term “alkylene,” as used herein, refers to a bivalent straight or branched saturated or unsaturated hydrocarbon chain. In some embodiments, an alkylene group is saturated.

In certain embodiments, exemplary aliphatic groups include, but are not limited to, ethynyl, 2-propynyl, 1-propenyl, 2-butenyl, 1,3-butadienyl, 2-pentenyl, vinyl (ethenyl), allyl, isopropenyl, methyl, ethyl, propyl, isopropyl, butyl, isobutyl, sec-butyl, tert-butyl, pentyl, isopentyl, sec-pentyl, neo-pentyl, tert-pentyl, cyclopentyl, hexyl, isohexyl, sec-hexyl, cyclohexyl, 2-methylpentyl, tert-hexyl, 2,3-dimethylbutyl, 3,3-dimethylbutyl, 1,3-dimethylbutyl, and 2,3-dimethyl but-2-yl.

The term “alkylidene,” as used herein, refers to a divalent group formed from an alkane by removal of two hydrogen atoms from the same carbon atom, the free valencies of which are part of a double bond. By way of nonlimiting example, an alkylidene may be of the formula ═C(R^{q})₂, ═CHR^{q}, or ═CH₂, wherein R^{q} represents any suitable substituent other than hydrogen.

The terms “haloalkyl,” “haloalkenyl” and “haloalkoxy” means alkyl, alkenyl or alkoxy, as the case may be, substituted with one or more halogen atoms. The term “halogen” means F, Cl, Br, or I. Such “haloalkyl,” “haloalkenyl” and “haloalkoxy” groups may have two or more halo substituents which may or may not be the same halogen and may or may not be on the same carbon atom. Examples include chloromethyl, periodomethyl, 3,3-dichloropropyl, 1,3-difluorobutyl, trifluoromethyl, and 1-bromo-2-chloropropyl.

The term “heterocycle,” “heterocyclyl,” “heterocycloaliphatic,” or “heterocyclic” as used herein means non-aromatic, monocyclic, bicyclic, or tricyclic ring systems in which one or more ring members is an independently selected heteroatom. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 7 ring members.

A heterocyclic ring can be attached to its pendant group at any heteroatom or carbon atom that results in a stable structure and, when specified, any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “alkoxy,” or “thioalkyl,” as used herein, refers to an alkyl group, as previously defined, attached to the principal carbon chain through an oxygen (“alkoxy”) or sulfur (“thioalkyl”) atom.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein one or more ring in the system is aromatic and wherein each ring in the system contains 3 to 7 ring members. The term “aryl” may be used interchangeably with the term “aryl ring”. The term “aryl” also refers to heteroaryl ring systems as defined hereinbelow. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The term “heteroaryl,” used alone or as part of a larger moiety as in “heteroaralkyl” or “heteroarylalkoxy,” refers to monocyclic, bicyclic, and tricyclic ring systems having a total of five to fourteen ring members, wherein one or more ring in the system is aromatic, one or more ring in the system contains one or more heteroatoms, and wherein each ring in the system contains 3 to 7 ring members. The term “heteroaryl” may be used interchangeably with the term “heteroaryl ring” or the term “heteroaromatic”. Heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl.

The terms “heteroaryl” and “heteroar-,” as used herein, also include groups in which a heteroaromatic ring is fused to one or more aryl, cycloaliphatic, or heterocyclyl rings. Exemplary heteroaryl rings include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄—R^{o}; —(CH₂)₀₋₄OR^{o}; —O(CH₂)₀₋₄R^{o}, —O—(CH₂)₀₋₄—C(O)OR^{o}; —(CH₂)₀₋₄—CH(OR^{o})₂; —(CH₂)₀₋₄SR^{o}; —(CH₂)₀₋₄Ph, which may be substituted with R^{o}; —(CH₂)₀₋₄—O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R., -(haloR.), —(CH₂)₀₋₂OH, —(CH₂)₀₋₂OR., —(CH₂)₀₋₂CH(OR.)₂; —O(haloR.), —CN, —N₃, —(CH₂)₀₋₂C(O)R., —(CH₂)₀₋₂C(O)OH, —(CH₂)₀₋₂C(O)OR., —(CH₂)₀₋₂SR., —(CH₂)₀₋₂SH, —(CH₂)₀₋₂NH₂, —(CH₂)₀₋₂NHR., —(CH₂)₀₋₂NR.₂, —NO.₂, —SiR.₁₃, —OSiR.₁₃, —C(O)SR., —(C₁₋₄ straight or branched alkylene)C(O)OR., or —SSR. wherein each R. is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, and ═C(R*)₂, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C— or ¹³C— or ¹⁴C-enriched carbon are within the scope of this invention. Such compounds are useful, for example, as analytical tools or probes in biological assays.

### BRIEF DESCRIPTION OF THE DRAWING

FIG. 1. LC-MS of compound C.

FIG. 2. LC-MS of E-1.

FIG. 3. ¹H NMR of compound E-2.

FIG. 4. ¹H NMR of compound E-2.

FIG. 5. LC-MS of compound E-2.

FIG. 6. a) ¹H NMR of compound E-3; b) ¹H NMR of compound E-3 (close-up).

FIG. 7. a) ¹H NMR of compound E-4; b) ¹H NMR of compound E-4 (close-up).

FIG. 8. a) ¹H NMR of compound E-5; b) ¹H NMR of compound E-5 (close-up).

FIG. 9. a) ¹H NMR of compound E-6; b) ¹H NMR of compound E-6 (close-up).

FIG. 10. a) ¹H NMR of compound E-7; b) ¹H NMR of compound E-7 (close-up).

FIG. 11. LC-MS of compound E-8.

FIG. 12. a) LC-MS of compound E-9; b) ¹H NMR of compound E-9.

FIG. 13. LC-MS of compound E-10.

FIG. 14. a) LC-MS of compound E-11; b) ¹H NMR of compound E-11.

FIG. 15. a) LC-MS of compound E-12; b) ¹H NMR of compound E-12.

FIG. 16. a) LC-MS of compound E-13; b) ¹H NMR of compound E-13.

FIG. 17. a) LC-MS of compound E-14; b) ¹H NMR of compound E-14.

FIG. 18. Exemplary synthesis.

FIG. 19. Exemplary synthesis.

FIG. 20. Exemplary synthesis.

FIG. 21. Exemplary synthesis.

### GENERAL METHODS

The compounds of this invention may be prepared or isolated in general by synthetic and/or semi-synthetic methods known to those skilled in the art for analogous compounds and by methods described in detail in the Examples, herein. Methods and intermediates of the present invention are useful for preparing compounds as described in, e.g. U.S. patent application Ser. No. 13/040,166, filed Mar. 3, 2011, in the name of Bronk et al., the entirety of which is incorporated herein by reference.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group of the R¹⁰ moiety is phthalimido. In still other embodiments, the amino protecting group of the R¹⁰ moiety is a tert-butyloxycarbonyl (BOC) group. In certain embodiments, the amino protecting group is a sulphone (SO₂R).

Each of R, R¹, R², L, PG¹, PG², and PG⁴ in the below Schemes is as defined and described in classes and subclasses herein.

Isolation of Material from Biomass

Certain compounds used in methods of the present invention are isolated from black cohosh root, also known as cimicifuga racemosa or actaea racemosa. Commercial extracts, powders, and capsules of black cohosh root are available for treating a variety of menopausal and gynecological disorders. However, it has been surprisingly found that certain compounds present in black cohosh root are useful for modulating and/or inhibiting amyloid-beta peptide production. In particular, certain compounds have been isolated from black cohosh root and identified, wherein these compounds are useful as syntheteic precursors en route to compounds useful for modulating and/or inhibiting amyloid-beta peptide production, and in particular amyloid-beta peptide (1-42). These compounds may be isolated and utilized in a form substantially free of other compounds normally found in the root.

In some embodiments, methods of the present invention for use in preparing a compound of formula II use compounds found in extracts of black cohosh and related cimicifuga species, whether from roots and rhizome or aerial parts of these plants. One of ordinary skill in the art will recognize that synthetic precursors may be obtained from one or more cimicifuga species including, but not limited to, Cimicifuga racemosa, Cimicifuga dahurica, Cimicifuga foetida, Cimicifuga heracleifolia, Cimicifuga japonica, Cimicifuga acerina, Cimicifuga acerima, Cimicifuga simplex, and Cimicifuga elata, Cimicifuga calthaefolia, Cimicifuga frigida, Cimicifuga laciniata, Cimicifuga mairei, Cimicifuga rubifolia, Cimicifuga americana, Cimicifuga biternata, and Cimicifuga bifida or a variety thereof. This may be accomplished either by chemical or biological transformation of an isolated compound or an extract fraction or mixture of compounds. Chemical transformation may be accomplished by, but not limited to, manipulation of temperature, pH, and/or treatment with various solvents. Biological transformation may be accomplished by, but not limited to, treatment of an isolated compound or an extract fraction or mixture of compounds with plant tissue, plant tissue extracts, other microbiological organisms or an isolated enzyme from any organism.

In some embodiments, a precursor compound is extracted from a sample of biomass to provide a compound of formula A, as depicted in Scheme I below.

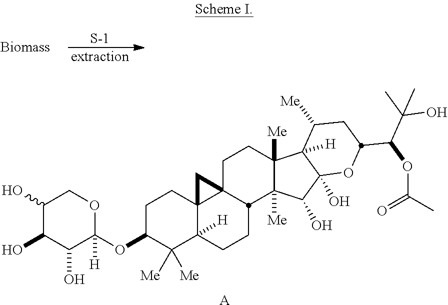

The term “biomass,” as used herein, refers to roots, rhizomes and/or aerial parts of the cimicifuga species of plant, as described above and herein.

In some embodiments, the process of obtaining a compound of formula A from biomass comprises a step of pre-treating the biomass. In some embodiments, the step of pretreating comprises a step of drying. In certain embodiments, the step of drying comprises use of one or more suitable methods for providing biomass of a desired level of dryness. For instance, in some embodiments the biomass is dried using vacuum. In some embodiments, the biomass is dried using heat. In some embodiments, the biomass is dried using a spray dryer or drum dryer. In some embodiments, the biomass is dried using two or more of the above methods.

In some embodiments, the step of pretreating comprises a step of grinding. In certain embodiments, the step of grinding comprises passing the sample of biomass through a chipper or grinding mill for an amount of time suitable to provide biomass of a desired particle size. In some embodiments, the biomass is dried prior to being ground to a suitable particle size.

In some embodiments, a suitable particle size ranges from about 0.1 mm³ to about 1.0 mm³. In some embodiments, a suitable particle size ranges from about 0.2 mm³ to about 1.0 mm³. In some embodiments, a suitable particle size ranges from about 0.3 mm³ to about 1.0 mm³. In some embodiments, a suitable particle size ranges from about 0.4 mm³ to about 1.0 mm³. In some embodiments, a suitable particle size ranges from about 0.5 mm³ to about 1.0 mm³. In some embodiments, a suitable particle size ranges from about 0.6 mm³ to about 1.0 mm³. In some embodiments, a suitable particle size ranges from about 0.7 mm³ to about 1.0 mm³. In some embodiments, a suitable particle size ranges from about 0.8 mm³ to about 1.0 mm³. In some embodiments, a suitable particle size ranges from about 0.9 mm³ to about 1.0 mm³.

In some embodiments, a suitable particle size ranges from about 0.1 mm³ to about 0.9 mm³. In some embodiments, a suitable particle size ranges from about 0.1 mm³ to about 0.8 mm³. In some embodiments, a suitable particle size ranges from about 0.1 mm³ to about 0.7 mm³. In some embodiments, a suitable particle size ranges from about 0.1 mm³ to about 0.6 mm³. In some embodiments, a suitable particle size ranges from about 0.1 mm³ to about 0.5 mm³. In some embodiments, a suitable particle size ranges from about 0.1 mm³ to about 0.4 mm³. In some embodiments, a suitable particle size ranges from about 0.1 mm³ to about 0.3 mm³. In some embodiments, a suitable particle size ranges from about 0.1 mm³ to about 0.2 mm³.

In some embodiments, biomass is dried and ground prior to being extracted. The term “extraction,” as used herein, refers to the general process of obtaining a compound of formula A comprising a step of exposing biomass to one or more suitable solvents under suitable conditions for a suitable amount of time in order to extract a compound of formula A from the biomass. In some embodiments, extraction comprises agitating and heating a slurry comprised of biomass and one or more suitable solvents. In certain embodiments, the one or more suitable solvents comprise one or more alcohols, and optionally water. Suitable alcohols include, but are not limited to, methanol, ethanol, isopropanol, and the like. In certain embodiments, the alcohol is methanol. In certain embodiments, the alcohol is ethanol. In some embodiments, the slurry is heated to a temperature of about 25° C., 30° C., 35° C., 40° C., 45° C., 50° C., 55° C., 60° C., 65° C., and 70° C. In some embodiments, an elevated temperature is a temperature of greater than about 70° C. In certain embodiments, the slurry is heated to about 50° C. In certain embodiments, the slurry is kept at ambient temperature.

In some embodiments, the biomass is exposed to one or more suitable solvents under suitable conditions for an amount of time ranging from about 0.1 h to about 48 h. In some embodiments, the amount of time ranges from about 0.1 h to about 36 h. In some embodiments, the amount of time ranges from about 0.1 h to about 24 h. In some embodiments, the amount of time ranges from about 0.5 h to about 24 h. In some embodiments, the amount of time ranges from about 1 h to about 24 h. In some embodiments, the amount of time ranges from about 2 h to about 24 h. In some embodiments, the amount of time ranges from about 2 h to about 22 h. In some embodiments, the amount of time ranges from about 2 h to about 20 h. In some embodiments, the amount of time ranges from about 2 h to about 4 h. In some embodiments, the amount of time ranges from about 20 h to about 24 h. In some embodiments, the amount of time is about 2 h. In some embodiments, the amount of time is about 22 h.

In some embodiments, once the slurry of biomass is heated and/or agitated for a suitable amount of time, the slurry is filtered through e.g., Celite, and concentrated down to the crude extract. In certain embodiments, the crude extract is further treated with an aqueous salt solution such as, e.g., 5% aqueous KCl, and cooled to a temperature of about 2° C. to about 10° C. Exemplary other salts for use in an aqueous salt solution include, but are not limited to, (NH₄)SO₄, K₂SO₄, NaCl, etc. In some embodiments, the aqueous salt solution has a concentration ranging from about 1% to about 50%. In some embodiments, the aqueous salt solution has a concentration ranging from about 3% to about 30%. In some embodiments, the aqueous salt solution has a concentration ranging from about 5% to about 10%. In some embodiments, the aqueous salt solution has a concentration ranging from about 10% to about 20%. In some embodiments, the aqueous salt solution has a concentration ranging from about 20% to about 30%. In certain embodiments, the crude extract is cooled to a temperature of about 2° C. to about 6° C. In certain embodiments, the crude extract is cooled to a temperature of about 4° C. In some embodiments, the crude extract is cooled for about 1, 2, 3, 4, or 5 h. In certain embodiments, the crude extract is cooled for about 2 h. In some embodiments, the crude extract is cooled for more than about 5 h. In certain embodiments, the crude extract is cooled for about 5 h to about 10 h. In certain embodiments, the crude extract is cooled for about 10 h to about 15 h. In certain embodiments, the crude extract is cooled for about 15 h to about 20 h. In certain embodiments, the crude extract is cooled for about 20 h to about 25 h. In some embodiments, after the crude extract is cooled for an appropriate amount of time, the slurry is centrifuged and the resulting solids are collected and dried using any one or more methods known in the art.

In other embodiments, the crude extract is partitioned between water and an organic solvent, such as DCM and the organic fraction is subsequently removed, concentrated, the solution is filtered through silica gel and then brought to dryness, affording compound A in about 3-15% purity.

In some embodiments, step S-1 provides compound A in about 3-15% purity.

In some embodiments, the present invention provides a method for obtaining a compound of formula A. In certain embodiments, the present invention provides a method for obtaining a compound of formula A from biomass comprising the step of contacting the biomass with one or more suitable solvents under suitable conditions for a suitable amount of time to obtain a compound of formula A.

### General Method for Preparing Compounds of Formula II

In some embodiments, compound A serves as starting material in the synthesis of a compound of formula II, as illustrated in Scheme II below.

As depicted in step S-2 of Scheme II, the hydroxyl moiety of formula A is treated with a suitable acid to provide carbonyl compound B, which, in step S-3 is oxidatively cleaved at the polyol moiety to afford dialdehyde C. In certain embodiments, the suitable acid is a Lewis acid or protic acid. In certain embodiments, the suitable acid is a Lewis acid. Exemplary syntheses of compounds of the present invention utilizing dialdehyde C are provided in the Exemplification section herein.

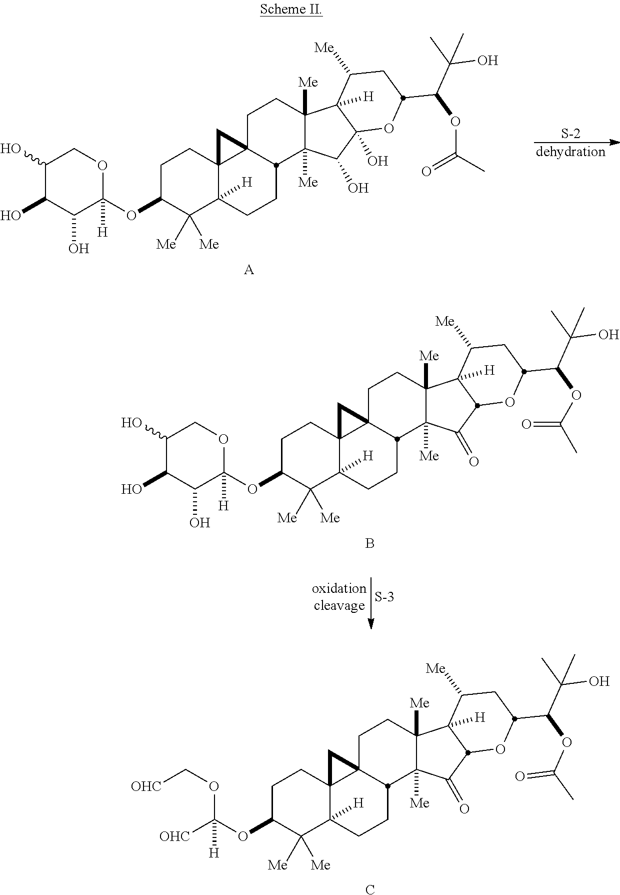

In some embodiments, the reductive amination of dialdehyde C in step S-4 provides morpholine D, as illustrated in Scheme III below. In step S-5, the carbonyl group of morpholine D generated in S-2 is reduced to the corresponding hydroxyl group to provide alcohol E, which is then protected in step S-6 with a suitable oxygen protecting group to afford F. The acetate moiety of F is then deacetylated in step S-7 to provide the corresponding free alcohol G. The newly deacetylated alcohol of G is then modified in step S-8 to provide H, which is then deprotected and further derivatized to afford a compound of formula II.

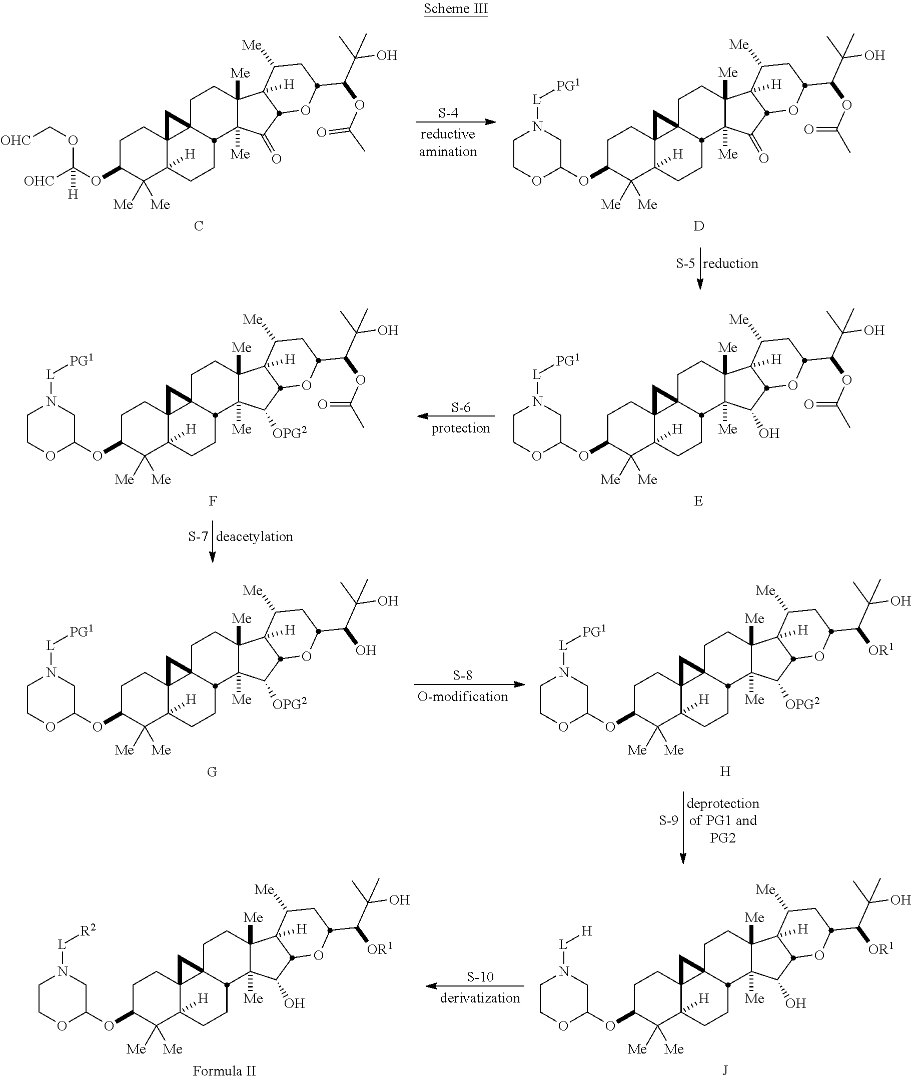

In some embodiments, the reductive amination of dialdehyde C in step S-4 provides morpholine D-i, as illustrated in Scheme IV below. In step S-5, the carbonyl group of morpholine D-i generated in S-2 is reduced to the corresponding hydroxyl group to provide alcohol E-i, which is then protected in step S-6 with a suitable oxygen protecting group to afford F-i. The acetate moiety of F-i is then deacetylated in step S-7 to provide the corresponding free alcohol G-i. The newly deacetylated alcohol of G-i is then modified in step S-8 to provide H-i, which is then deprotected to provide a compound of formula I.

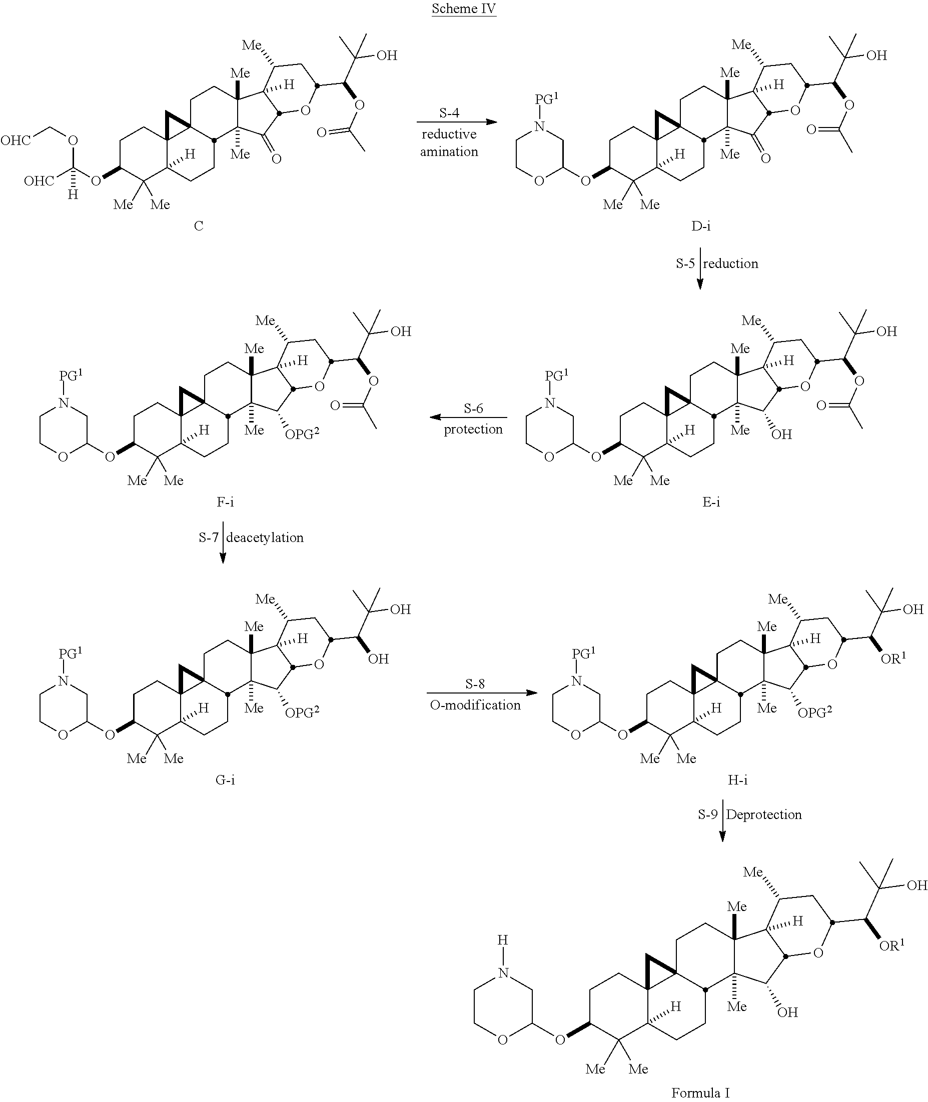

One of skill in the art will recognize that a variety of compounds of formula II can be accessed via formula I using any one or more methods known in the art to effect modifications at the nitrogen atom of the morpholine ring of formula I. Exemplary such reactions include, but are not limited to, alkylation reactions, acylation reactions, cross-coupling reactions, etc.

In some embodiments, a compound of formula II is prepared from 1 in a single step as outlined in Scheme V below.

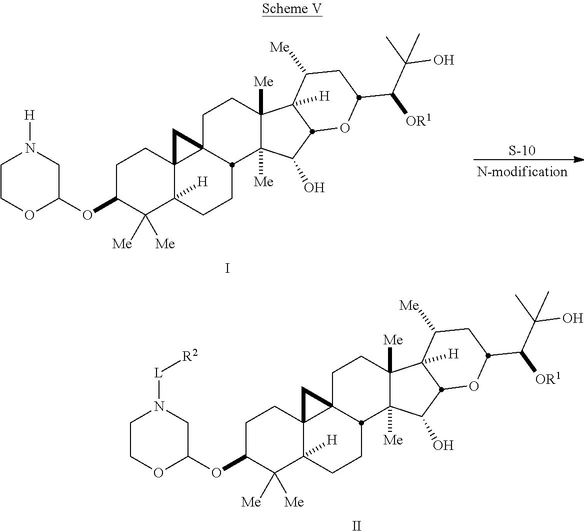

In some embodiments, a compound of formula II is prepared from a compound of formula I in multiple steps as outlined in Scheme VI below.

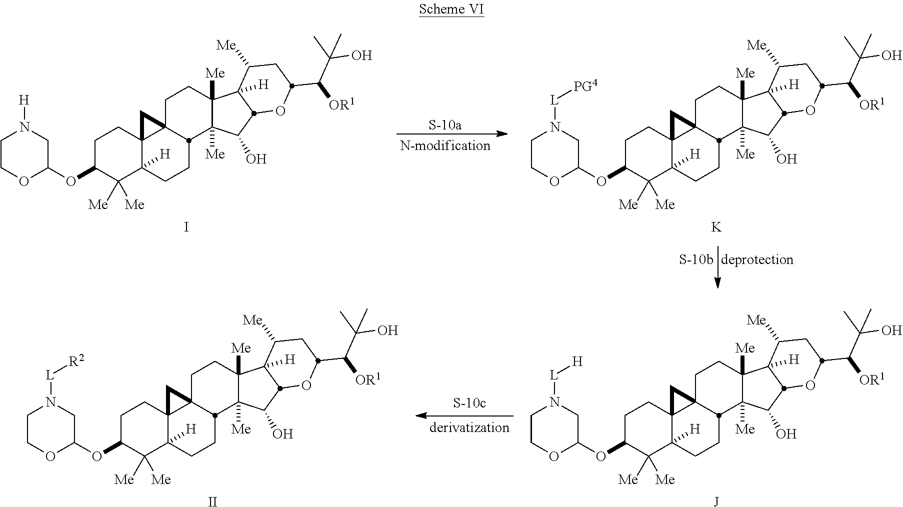

In some embodiments, the reductive amination of dialdehyde C in step S-4 provides morpholine D-ii, as illustrated in Scheme VII below. In step S-5, the carbonyl group of morpholine D-ii is reduced to the corresponding hydroxyl group to provide alcohol E-ii. Deprotection of the acetyl group takes place in step S-6 without the need for protecting the hydroxyl group provided in step S-5 which is followed by oxygen modification in step S-7 to provide the compound of formula II.

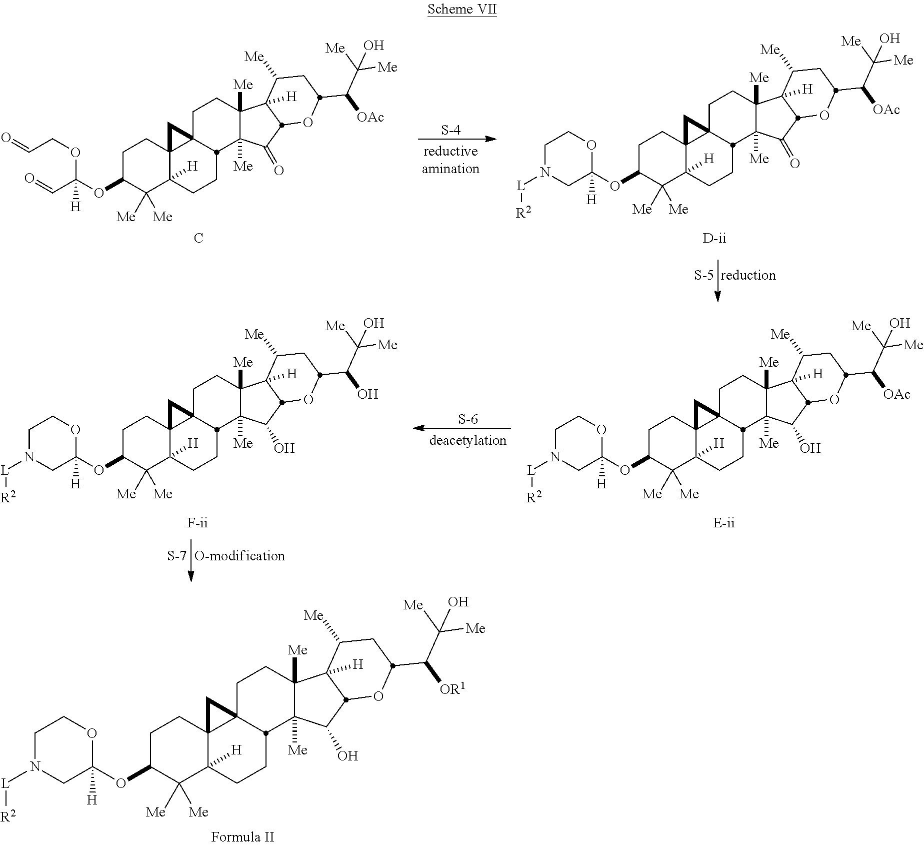

### Description of Synthetic Steps

As depicted in step S-2 above, exposure of compound A to an acid under suitable conditions provides carbonyl compound B. In some embodiments, the acid is a Lewis acid (e.g., ZrCl₄). In some embodiments, the reaction occurs in a chlorinated solvent such as chloroform or methylene chloride. In certain embodiments, compound A is dissolved in methylene chloride and an amount of Lewis acid (or solution thereof) is added in portions over time. In some embodiments, the Lewis acid is added in three portions over the course of 1, 2, or 3 hours. In certain embodiments, the Lewis acid is added in three portions over the course of 1 hour and the reaction is run at ambient temperature. In some embodiments, ambient temperature is 20° C.

In certain embodiments, the reaction of S-1 occurs in the presence of a base. In some embodiments, S-1 occurs in the presence of an amine base, such as triethylamine.

In some embodiments, crude compound A is taken on to step S-2 without further purification. In some embodiments, crude compound A is pretreated prior to step S-2. For instance, in certain embodiments, compound A is dissolved in a polar aprotic solvent (e.g., DMSO) and filtered through a solid phase (e.g., Celite). In some embodiments, the filtered compound A is further purified prior to step S-2 using chromatography (e.g., reverse phase chromatography). Exemplary such methods are described in the exemplification section herein. In some embodiments, purification provides compound A in about 50, 60, 70, or 80% purity.

As depicted in step S-3 above, the polyol of compound B is oxidatively cleaved upon exposure to a suitable oxidant to afford dialdehyde C. In some embodiments, a suitable oxidant is a hypervalent iodide. In certain embodiments, the oxidant is sodium periodate and the solvent is a mixture of an organic solvent and an aqueous solvent. In some embodiments, the organic solvent is an ethereal solvent such as a tetrahydrofuran or a dialkyl ether. In some embodiments, the solvent mixture comprises an ethereal solvent and water in a v/v ratio of 5:1, 4:1, 3:1, 2:1, 1:1, 1:2, 1:3, 1:4, or 1:5. In certain embodiments, the solvent mixture comprises THF and water in a v/v ratio of 3:1. In some embodiments, suitable conditions for cleaving the polyol include heating the reaction for a suitable amount of time until TLC analysis indicates that the reaction is complete. In some embodiments, the reaction is run at ambient temperature. In some embodiments, the reaction is heated to about 30° C., 35° C., 40° C., 45° C., 50° C., 55° C., 60° C., 65° C., or 70° C. In certain embodiments, the reaction is heated to about 50° C. In some embodiments the reaction is heated for about 10 hours to about 20 hours. In certain embodiments the reaction is heated for about 15-17 hours.

As depicted in step S-4 above, dialdehyde C undergoes reductive amination in the presence of a suitable amine salt to provide a compound of formula D (see Scheme II above) or D-i (see Scheme III above). One of skill in the art will appreciate that the structure of the product of the reaction will be dictated by the structure of the amine salt reagent selected. For instance, in some embodiments, the amine salt is of the general formula shown below:

PG¹-L-NH₃⁺Cl⁻

wherein L is as defined and described herein and is other than a valence bond, PG¹ is any suitable protecting group, and the product of step S-4 is a compound of formula D. In some embodiments, the amine salt is of the general formula PG¹-NH₃⁺Cl⁻ and the product of step S-4 is a compound of formula D-i. In some embodiments, the PG¹ is a Boc or benzyl protecting group and the compound is of the formula D-i. In certain embodiments, the PG¹ is a BOC protecting group, L is other than a valence bond, and the reductive amination forms a compound of formula D.

In some embodiments, the amine salt of step S-4 is commercially available. In some embodiments, the amine salt of step S-4 is generated immediately prior to the reductive amination reaction taking place. For instance, in certain embodiments the amine salt is generated by dissolving an amine in a suitable solvent and adding said solvent to an aqueous solution of a desired acid (e.g., aqueous HCl) which, upon removal of the solvent mixture, affords the corresponding amine HCl salt for use in the reductive amination. In certain embodiments, a suitable solvent for generation of the amine is an alcoholic solvent such as ethanol. In certain embodiments, a suitable solvent for generation of the amine is a mixture of two or more alcoholic solvents, such as ethanol, methanol, and isopropanol. In some embodiments, the mixture is stirred for an amount of time and the solvent is removed at ambient temperature to provide the desired amine salt. In some embodiments, the solvent is removed at elevated temperatures to provide the desired amine salt. Methods of making amine salts are known in the chemical arts and described herein in the Exemplification.

In some embodiments, a reaction solvent for use in the reductive amination of step S-4 is a polar protic solvent. In certain embodiments, the polar protic solvent is an alcoholic solvent such as ethanol. In some embodiments, dialdehyde C is premixed with the amine salt in the presence of an acid. In certain embodiments, the acid is acetic acid and the mixture is stirred for about 10, 15, 20, 25, or 30 minutes prior to addition of the reducing agent. In some embodiments, the reducing agent is a borohydride reducing agent such as, e.g., NaBH(OAc)₃ and is used in molar excess with respect to the amount of amine salt present. In some embodiments, the reaction is allowed to proceed for 1, 2, 3, 4, or 5 hours, or until TLC analysis indicates completion. In some embodiments, upon reaction completion the product is treated to remove residual acid (e.g., via a toluene azeptrope), dried under vacuum, and carried on without further purification.

As depicted in step S-5 above, the carbonyl moiety of D or D-i is reduced upon exposure to sodium borohydride to afford alcohol E or E-i, respectively. In certain embodiments, sodium borohydride is premixed in a suitable solvent until at least partially dissolved. Exemplary such solvents include polar protic solvents (e.g., ethanol). In some embodiments, D or D-i is dissolved separately in a polar aprotic solvent such as ethyl acetate and added to the sodium borohydride reaction mixture over a period of time. In certain embodiments, D or D-i is added over a period of 1, 2, 3, 4, 5, 6, 7, 8, 9, or 10 minutes. In certain embodiments, the reaction is run at ambient temperature for an amount of time of about 5, 10, 15, 20, 25, 30, 35, 40 or 45 minutes. In some embodiments, the reaction is quenched with an acid (e.g., acetic acid) and the product is treated to remove residual acid (via e.g., a toluene azeotrope), dried under vacuum, and purified before being used in the next step.

As depicted in step S-6 above, the alcohol of E or E-i is protected to provide a compound of formula F or F-i using any suitable oxygen protecting group known in the art. In certain embodiments, the oxygen protecting group is a silyl protecting group (e.g., Et₃SiCl), the silylating reagent is used in excess, and a base is present. In some embodiments, the base is an amine base. In certain embodiments, the base is imidazole. In some embodiments, about 1.1 equivalents of silylating reagent are used relative to substrate. In some embodiments, about 1.5, 2, 2.5, 3, 3.5, 4, 4.5, or 5 equivalents of silylating reagent are used relative to substrate. In certain embodiments, the amount of silylating reagent required for a particular sample lot will be determined immediately prior to the reaction of that sample lot. For instance, in some embodiments, a sub-gram scale trial run is completed to gauge the purity of the lot. In certain embodiments wherein the reaction is a silylation reaction the solvent employed in the reaction is a polar aprotic solvent. In certain embodiments, the polar aprotic solvent is an amide-containing solvent such as dimethylformamide (DMF). In some embodiments, a reaction is run at ambient temperature for an amount of time of about 5, 10, 15, 20, 25, or 30 minutes. In some embodiments, a reaction is run at ambient temperature for an amount of time of about 30, 45, 60, 75, or 90 minutes. In some embodiments, a reaction is run at ambient temperature for about 1, 2, or 3 hours.

As depicted in step S-7 above, in some embodiments, a compound of formula F or F-i is deacylated at C-24 under basic conditions to provide an alcohol of formula G or G-i. In certain embodiments, the base is a carbonate base (e.g., K₂CO₃) and is used in excess relative to substrate. In some embodiments, the substrate is dissolved in an organic solvent that is a halogenated solvent (e.g., methylene chloride). In some embodiments, the substrate is dissolved in a polar protic solvent (e.g., methanol or ethanol). In certain embodiments, the solvent is a mixture of two or more solvents selected from at least one halogenated solvent and at least one polar protic solvent (e.g., methylene chloride and methanol). In some embodiments, a reaction is run at ambient temperature for an amount of time of about 1, 2, 3, 4, 5, 6, 7, or 8 hours. In certain embodiments, the reaction is run for about 2 hours. In certain embodiments, the reaction is run for about four hours. In certain embodiments, the product is worked up and carried on without further purification.

As depicted in step S-8 above, in some embodiments, alcohol G or G-i is modified to provide a compound of formula H using any one or more methods known in the art or described herein to modify a secondary hydroxyl group. Exemplary such methods include acylation, alkylation, and the like. In some embodiments, a compound of formula G or G-i is alkylated to provide an ether of formula H. In certain embodiments, a methylating reagent is used to afford the methyl ether. In certain embodiments, an ethylating reagent is used to provide an ethyl ether.

In some embodiments, alcohol G of G-i is alkylated using an alkyl halide, or equivalent thereof, in the presence of a base. In certain embodiments, the base is a hydride base, such as NaH. Exemplary alkyl halides include methyl bromide, ethyl bromide, methyl iodide, ethyl iodide, and the like. In some embodiments, the alkylating agent is ethyl iodide. In some embodiments, the alcohol to be alkylated is dissolved in a suitable solvent and pretreated with the base. For example, in certain embodiments, the alcohol is dissolved in a polar aprotic solvent (e.g., DMF) and treated with a hydride base (e.g., NaH) for 1, 2, 3, 4, 5, 6, 7, 8, 9, or 10 minutes prior to addition of the alkylating agent. In some embodiments, pretreatment with base occurs at reduced temperatures (e.g., about 0° C.). In some embodiments, the reaction is run for about 10, 20, 30, 40, 50, or 60 minutes or until TLC analysis indicates completion, whereupon the reaction is quenched at reduced temperatures (e.g., 0° C.) and purified to provide a compound of formula H or H-i.

In some embodiments, the deprotection step of S-9 occurs in a single step. In certain embodiments, the compound is of formula H and each of the oxygen and amine protecting groups are removed in a single step. In some embodiments, a compound of formula H is dissolved in a polar protic solvent and exposed to an acid under conditions suitable to deprotect both protecting groups. In certain embodiments, the polar protic solvent is an alcoholic solvent (e.g., methanol) and the acid is Bronsted acid such as aqueous HCl. In certain embodiments, the polar protic solvent is a chlorinated solvent (e.g., methylene chloride) and the acid is an organic acid such as TFA. In some embodiments, the reaction occurs at elevated temperatures of about 30, 40, 50, or 60° C. In certain embodiments, the reaction occurs at 50° C. In some embodiments, the reaction occurs at room temperature.

In some embodiments, the deprotection step of S-9 occurs in more than one step. For instance, in some embodiments, removal of the oxygen protecting group and the amine protecting group is iterative.

One of skill in the art would appreciate that various methods for removal of protecting groups are known in the chemical arts and, in particular, can be found in Protecting Groups in Organic Synthesis, T. W. Greene and P. G. M. Wuts, 3^{rd} edition, John Wiley & Sons, 1999 referenced above.

In some embodiments, step S-10 occurs in one step as illustrated in Scheme V, above. For instance, in some embodiments, formula I undergoes N-alkylation to provide a compound of formula II. In some embodiments, formula I undergoes a reductive amination with a suitable carbonyl-containing compound to provide a compound of formula II.

In some embodiments, step S-10 occurs in more than one step as is illustrated in Scheme VI, above. In some embodiments, a compound is N-alkylated in step S-10a at the free amine of the morpholine ring to provide a compound of formula K. In certain embodiments, the N-alkylating reagent comprises a protecting group that requires subsequent removal. In certain embodiments, deprotection provides a compound of formula J. Formula J is then derivatized using any one or more suitable methods known in the art and/or methods described herein to provide a compound of formula II.

In some embodiments, the amine salt of step S-4 in Scheme VII is generated prior to the reductive amination reaction taking place. For instance, in certain embodiments the amine salt is generated by dissolving an amine salt in a suitable solvent and adding said solvent to an aqueous solution of a desired acid (e.g., aqueous HCl) which, upon removal of the solvent mixture, affords the corresponding amine HCl salt for use in the reductive amination. In certain embodiments, a suitable solvent for generation of the amine is an alcoholic solvent such as ethanol. In certain embodiments, a suitable solvent for generation of the amine is a mixture of two or more alcoholic solvents, such as ethanol, methanol, and isopropanol. In some embodiments, the mixture is stirred for an amount of time and the solvent is removed at ambient temperature to provide the desired amine salt. In some embodiments, the solvent is removed at elevated temperatures to provide the desired amine salt. Methods of making amine salts are known in the chemical arts and described herein in the Exemplification.

In some embodiments, a reaction solvent for use in the reductive amination of step S-4 of Scheme VII is a polar protic solvent. In certain embodiments, the polar protic solvent is an alcoholic solvent such as ethanol. In some embodiments, dialdehyde C is premixed with the amine salt in the presence of an acid. In certain embodiments, the acid is acetic acid and the mixture is stirred for about 10, 15, 20, 25, or 30 minutes prior to addition of the reducing agent. In some embodiments, the reducing agent is a borohydride reducing agent such as, e.g., NaBH₃(CN). In some embodiments, the reaction is allowed to proceed for 1, 2, 3, 4, or 5 hours, or until TLC analysis indicates completion. In some embodiments, upon reaction completion the product is treated to remove residual acid (e.g., via a toluene azeotrope), dried under vacuum, and carried on without further purification.

As depicted in step S-5 of Scheme VII above, the carbonyl moiety of D-ii is reduced upon exposure to sodium borohydride to afford alcohol E-ii. In certain embodiments, sodium borohydride is premixed in a suitable solvent until at least partially dissolved. Exemplary such solvents include polar protic solvents (e.g., ethanol). In some embodiments, D-ii is dissolved separately in a polar aprotic solvent such as ethyl acetate and added to the sodium borohydride reaction mixture over a period of time. In certain embodiments, D-ii is added over a period of 1, 2, 3, 4, 5, 6, 7, 8, 9, or 10 minutes. In certain embodiments, the reaction is run at ambient temperature for an amount of time of about 5, 10, 15, 20, 25, 30, 35, 40, 45, 50, 55, 60, 65, 70, 75, 80, 85, or 90 minutes. In some embodiments, the reaction is quenched with an acid (e.g., acetic acid) and the product is treated to remove residual acid (via e.g., a toluene azeotrope), dried under vacuum, and purified before being used in the next step.

As depicted in step S-6 of Scheme VII above, the reduced alcohol of E-ii is not protected in subsequent steps. One of ordinary skill in the art will appreciate that in some embodiments the reduced alcohol of E-ii can be protected with a suitable hydroxyl protecting group and the resulting intermediate taken on to subsequent steps of the synthesis.

As depicted in step S-6 of Scheme VII above, in some embodiments, a compound of formula E-ii is deacetylated at C-24 under basic conditions to provide an alcohol of formula F-ii. In certain embodiments, the base is a hydroxide base (e.g., NaOH) and is used in excess relative to substrate. In some embodiments, the substrate is dissolved in an organic solvent that is a halogenated solvent (e.g., methylene chloride). In some embodiments, the substrate is dissolved in a polar protic solvent (e.g., methanol or ethanol). In certain embodiments, the solvent is a mixture of two or more solvents selected from at least one halogenated solvent and at least one polar protic solvent (e.g., methylene chloride and methanol). In some embodiments, a reaction is run at ambient temperature for an amount of time of about 1, 2, 3, 4, 5, 6, 7, or 8 hours. In certain embodiments, the reaction is run for about 5 hours. In certain embodiments, the reaction is run for about four hours. In certain embodiments, the product is worked up and carried on without further purification.

As depicted in step S-7 of Scheme VII above, alcohol F-ii is alkylated using an alkylating agent in the presence of a base. In certain embodiments, the alkylating agent is an alkyl halide or alkoxy sulfoxide. In certain embodiments, the base is a hydride base, such as NaH, or an alkoxy base, such as a NaOtBu. Exemplary alkyl halides include methyl bromide, ethyl bromide, methyl iodide, ethyl iodide, and the like. Exemplary alkoxy sulfoxides include (MeO)₂SO₂, or (EtO)₂SO₂. In some embodiments, the alkylating agent is ethyl iodide. In some embodiments, the alcohol to be alkylated is dissolved in a suitable solvent and pretreated with the base.

### Methods

According to one aspect, the present invention provides a method for preparing a compound of formula II:

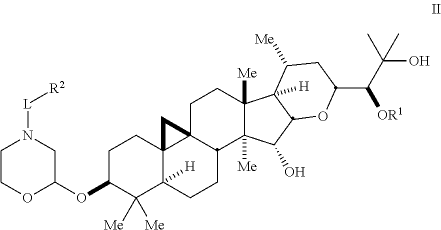

or a pharmaceutically acceptable salt thereof, wherein:
R¹ is R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R.

In some embodiments, the present invention provides a method for preparing a compound of formula II:

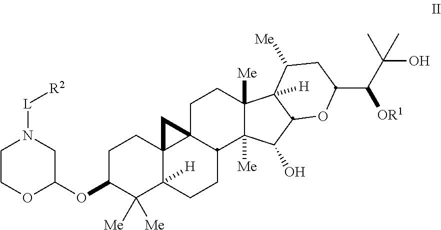

or a pharmaceutically acceptable salt thereof, wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R;
   comprising the steps of:
(a) extracting from biomass a compound of formula A:

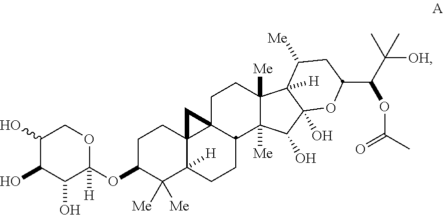

(b) treating said compound of formula A with a suitable acid to form a compound of formula B:

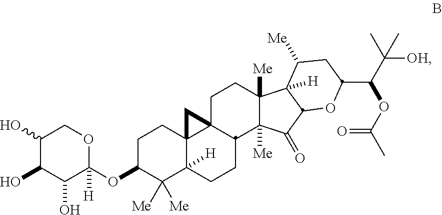

(c) treating said compound of formula B with a suitable oxidant to provide a compound of formula C:

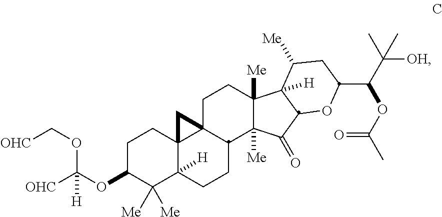

(d) treating said compound of formula C with a suitable amine, or salt thereof, in the presence of a base to provide a compound of formula D:

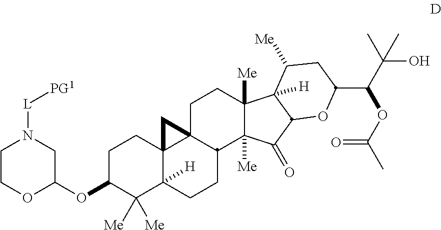

wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group;
(e) reducing the carbonyl component of said compound of formula D to form a compound of formula E:

wherein:

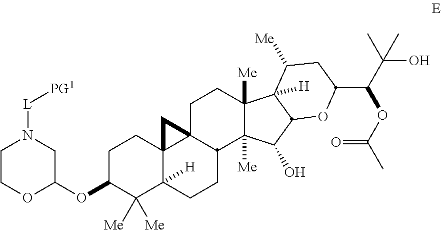

L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group;
(f) protecting said compound of formula E to form a compound of formula F:

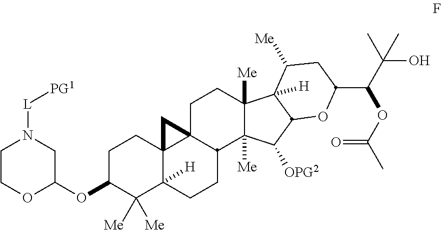

wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(g) deacetylating said compound of formula F to form a compound of formula G:

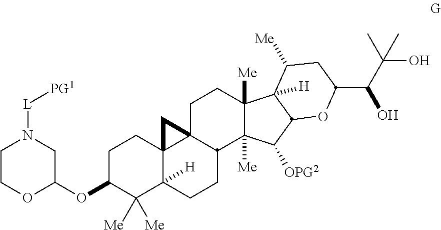

wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(h) reacting said compound of formula G to form a compound of formula H:

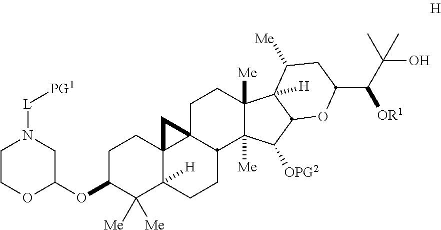

wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(i) deprotecting said compound of formula H to form a compound of formula J:

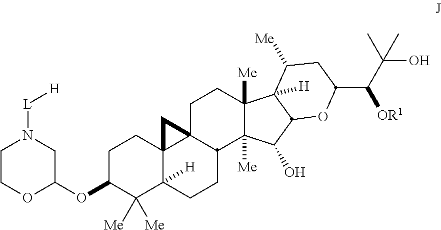

wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
(j) reacting said compound of formula J under suitable conditions to form a compound of formula II.

In some embodiments, the present invention provides a method for preparing a compound of formula II:

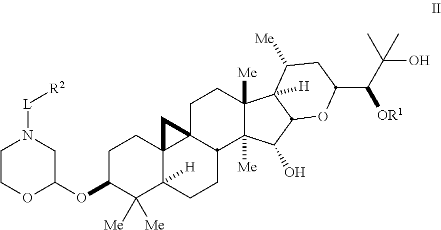

or a pharmaceutically acceptable salt thereof, wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R.
   comprising the steps of:
(a) extracting from biomass a compound of formula A:

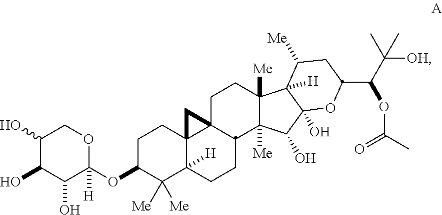

(b) treating said compound of formula A with a suitable acid to form a compound of formula B:

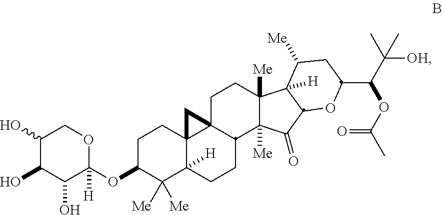

(c) treating said compound of formula B with a suitable oxidant to provide a compound of formula C:

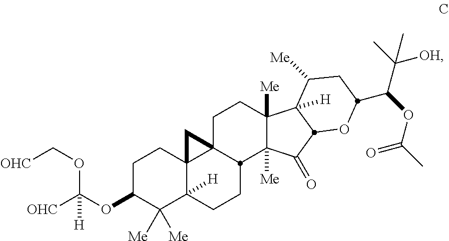

(d) treating said compound of formula C with a suitable amine, or salt thereof, in the presence of a base to provide a compound of formula D-1:

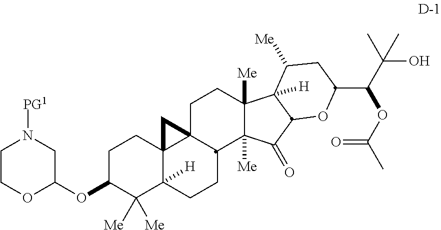

wherein:
PG¹ is a suitable amino protecting group;
(e) reducing the carbonyl component of said compound of formula D-1 to form a compound of formula E-1:

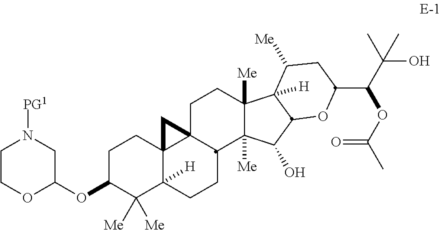

wherein:
PG¹ is a suitable amino protecting group;
(f) protecting said compound of formula E-1 to form a compound of formula F-1:

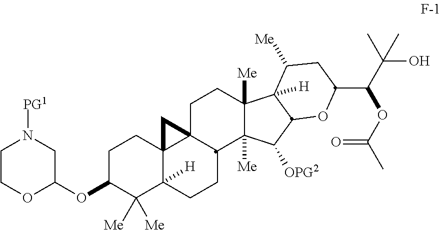

wherein:
PG¹ is a suitable amino protecting group; and
PG² is a suitable oxygen protecting group;
(g) deacetylating said compound of formula F-1 to form a compound of formula G-1:

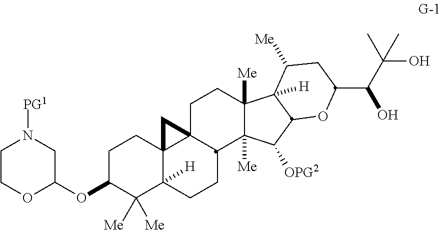

wherein:
PG¹ is a suitable amino protecting group; and
PG² is a suitable oxygen protecting group;
(h) reacting said compound of formula G-1 to form a compound of formula H-1:

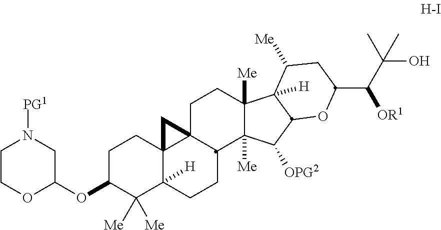

wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(i) deprotecting said compound of formula H-1 to form a compound of formula I:

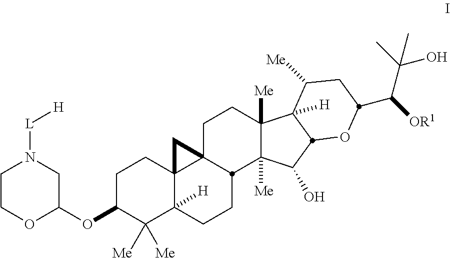

wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
(j) reacting said compound of formula I under suitable conditions to form a compound of formula II.

In certain embodiments, the formation of a compound of formula II from a compound of formula I comprises steps of:
(i) reacting a compound of formula I under suitable conditions to provide a compound of formula K:

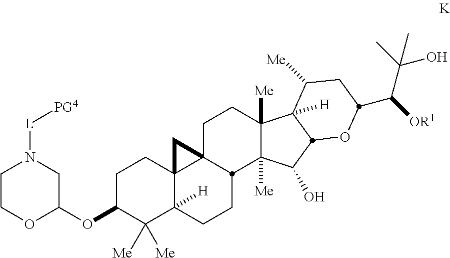

wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
and PG⁴ is an amino protecting group;
(ii) deprotecting a compound of formula K to provide a compound of formula J:

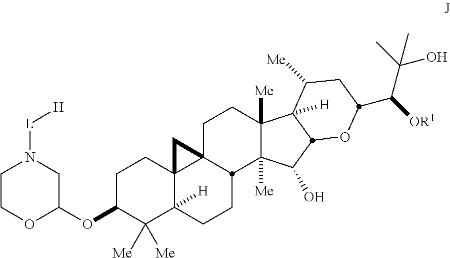

wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
(iii) reacting a compound of formula J to provide a compound of formula II.

In some embodiments, the present invention provides a method for preparing a compound of formula II:

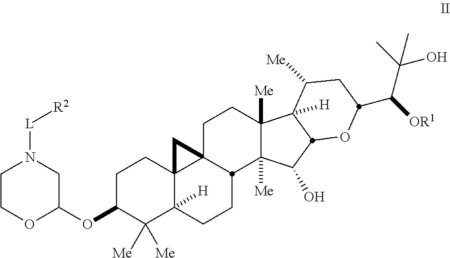

or a pharmaceutically acceptable salt thereof, wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R;
   comprising the steps of:
(a) extracting from biomass a compound of formula A:

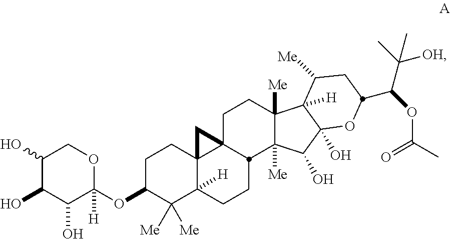

(b) treating said compound of formula A with a suitable acid to form a compound of formula B:

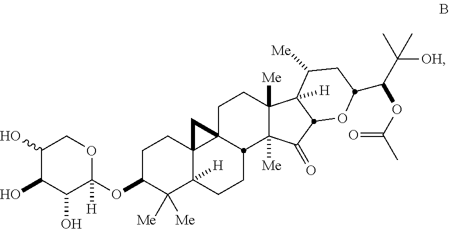

(c) treating said compound of formula B with a suitable oxidant to provide a compound of formula C:

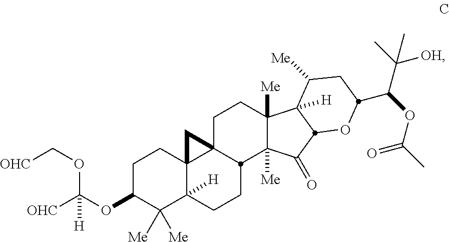

(d) treating said compound of formula C with a suitable amine, or salt thereof, in the presence of a base to provide a compound of formula D-2:

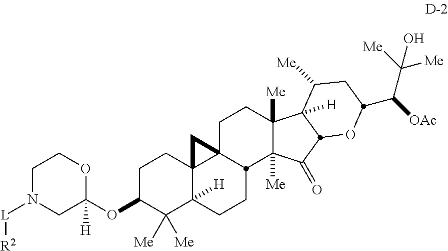

wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
R² is halogen or R;
(e) reducing the carbonyl component of said compound of formula D-2 to form a compound of formula E-2:

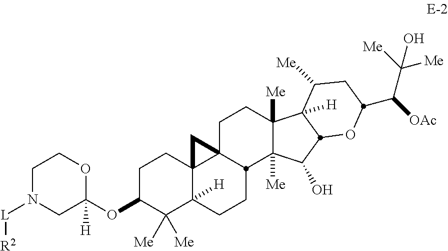

wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
R² is halogen or R;
(f) deacetylating said compound of formula E-2 to form a compound of formula F-2:

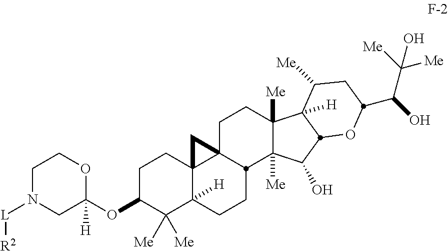

wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
R² is halogen or R;
(h) reacting said compound of formula F-2 under suitable conditions to form a compound of formula II.

In some embodiments, step (i) above is an N-alkylation reaction.

In some embodiments, step (iii) above is an N-alkylation reaction.

As defined generally above, R¹ is R, S(O)R, SO₂R, C(O)R, CO₂R, C(O)N(R)₂, or an optionally substituted aliphatic group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur.

In certain embodiments, R¹ is hydrogen. In certain embodiments, R¹ is optionally substituted C₁₋₁₀ aliphatic. In certain embodiments, R¹ is optionally substituted methyl, ethyl, propyl, or butyl. In certain embodiments, R¹ is an oxygen protecting group.

In some embodiments, R¹ is methyl or ethyl.

In certain embodiments, R¹ is an optionally substituted 3-8 membered saturated monocyclic ring having 1-3 heteroatoms independently selected from nitrogen, oxygen, or sulfur. In certain embodiments, R¹ is an optionally substituted 3-8 membered saturated monocyclic carbocycle. In certain embodiments, R¹ is an optionally substituted 5-6 membered saturated monocyclic ring having 1-2 heteroatoms independently selected from nitrogen, oxygen, or sulfur. In certain embodiments, R¹ is an optionally substituted 5-6 membered saturated monocyclic carbocycle. In certain embodiments, R¹ is an optionally substituted 7 membered saturated monocyclic ring having 1-2 heteroatoms independently selected from nitrogen, oxygen, or sulfur. In certain embodiments, R¹ is an optionally substituted 7 membered saturated monocyclic carbocycle.

Exemplary R¹ saturated 3-8 membered optionally substituted heterocycles include oxirane, oxetane, tetrahydrofuran, tetrahydropyran, oxepane, aziridine, azetidine, pyrrolidine, piperidine, azepane, thiirane, thietane, tetrahydrothiophene, tetrahydrothiopyran, thiepane, dioxolane, oxathiolane, oxazolidine, imidazolidine, thiazolidine, dithiolane, dioxane, morpholine, oxathiane, piperazine, thiomorpholine, dithiane, dioxepane, oxazepane, oxathiepane, dithiepane, diazepane, dihydrofuranone, tetrahydropyranone, oxepanone, pyrrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In certain embodiments, R¹ is an optionally substituted 3-8 membered partially unsaturated monocyclic ring having 1-3 heteroatoms independently selected from nitrogen, oxygen, or sulfur. In certain embodiments, R¹ is an optionally substituted 3-8 membered partially unsaturated monocyclic carbocycle. In certain embodiments, R¹ is an optionally substituted 5-6 membered partially unsaturated monocyclic ring having 1-2 heteroatoms independently selected from nitrogen, oxygen, or sulfur. In certain embodiments, R¹ is an optionally substituted 5-6 membered partially unsaturated monocyclic carbocycle. In certain embodiments, R¹ is an optionally substituted 5-6 membered aryl ring having 0-3 heteroatoms independently selected from nitrogen, oxygen, or sulfur. In certain embodiments, R¹ is an optionally substituted 5 membered aryl ring having 1-3 heteroatoms independently selected from nitrogen, oxygen, or sulfur. In certain embodiments, R¹ is an optionally substituted 6 membered aryl ring having 1-3 heteroatoms independently selected from nitrogen, oxygen, or sulfur. In certain embodiments, R¹ is an optionally substituted phenyl.

In certain embodiments, R¹ is an optionally substituted 8-10 membered saturated bicyclic ring having 0-4 heteroatoms independently selected from nitrogen, oxygen, or sulfur. In certain embodiments, R¹ is an optionally substituted 8 membered saturated bicyclic ring having 1-3 heteroatoms independently selected from nitrogen, oxygen, or sulfur. In certain embodiments, R¹ is an optionally substituted 8 membered saturated bicyclic carbocycle. In certain embodiments, R¹ is an optionally substituted 9 membered saturated bicyclic ring having 1-3 heteroatoms independently selected from nitrogen, oxygen, or sulfur. In certain embodiments, R¹ is an optionally substituted 9 membered saturated bicyclic carbocycle. In certain embodiments, R¹ is an optionally substituted 10 membered saturated bicyclic ring having 1-3 heteroatoms independently selected from nitrogen, oxygen, or sulfur. In certain embodiments, R¹ is an optionally substituted 10 membered saturated bicyclic carbocycle.

In certain embodiments, R¹ is an optionally substituted 8-10 membered partially unsaturated bicyclic ring having 0-4 heteroatoms independently selected from nitrogen, oxygen, or sulfur. In certain embodiments, R¹ is an optionally substituted 8 membered partially unsaturated bicyclic ring having 1-3 heteroatoms independently selected from nitrogen, oxygen, or sulfur. In certain embodiments, R¹ is an optionally substituted 8 membered partially unsaturated bicyclic carbocycle. In certain embodiments, R¹ is an optionally substituted 9 membered partially unsaturated bicyclic ring having 1-3 heteroatoms independently selected from nitrogen, oxygen, or sulfur. In certain embodiments, R¹ is an optionally substituted 9 membered partially unsaturated bicyclic carbocycle. In certain embodiments, R¹ is an optionally substituted 10 membered partially unsaturated bicyclic ring having 1-3 heteroatoms independently selected from nitrogen, oxygen, or sulfur. In certain embodiments, R¹ is an optionally substituted 10 membered partially unsaturated bicyclic carbocycle.

In certain embodiments, R¹ is an optionally substituted 9-10 membered aryl bicyclic ring having 0-4 heteroatoms independently selected from nitrogen, oxygen, or sulfur. In certain embodiments, R¹ is an optionally substituted 9 membered aryl bicyclic ring having 1-4 heteroatoms independently selected from nitrogen, oxygen, or sulfur. In certain embodiments, R¹ is an optionally substituted 9 membered aryl bicyclic ring having 3 heteroatoms independently selected from nitrogen, oxygen, or sulfur. In certain embodiments, R¹ is an optionally substituted 9 membered aryl bicyclic ring having 2 heteroatoms independently selected from nitrogen, oxygen, or sulfur. In certain embodiments, R¹ is an optionally substituted 9 membered aryl bicyclic ring having 1 heteroatom selected from nitrogen, oxygen, or sulfur. In certain embodiments, R¹ is an optionally substituted 10 membered aryl bicyclic ring having 0-3 heteroatoms independently selected from nitrogen, oxygen, or sulfur. In certain embodiments, R¹ is an optionally substituted 10 membered aryl bicyclic ring having 1-2 heteroatoms independently selected from nitrogen, oxygen, or sulfur. In certain embodiments, R¹ is an optionally substituted naphthyl.

Exemplary optionally substituted R¹ heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one, or chromanyl.

As defined generally above and herein, L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur.

In some embodiments, L is a valence bond.

In some embodiments, L is an optionally substituted C₁₋₁₀ alkylene chain wherein one, two, or three methylene units are independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-.

In certain embodiments, L is an optionally substituted C₁₋₁₀ alkylene chain wherein one, two, or three methylene units are independently replaced by -Cy-.

In certain embodiments, L is an optionally substituted C₁₋₁₀ alkylene chain wherein one methylene unit is independently replaced by -Cy-.

In certain embodiments, L is an optionally substituted C₁₋₁₀ alkylene chain wherein two methylene units are independently replaced by -Cy-.

In certain embodiments, L is an optionally substituted C₁₋₁₀ alkylene chain wherein three methylene units are independently replaced by -Cy-.

In some embodiments, L is an optionally substituted C₂₋₁₀ alkylene chain wherein one or more methylene unit is independently replaced by -Cy-, and wherein one or more -Cy- is independently a bivalent optionally substituted saturated monocyclic ring. In some embodiments, one or more -Cy- is independently a bivalent optionally substituted partially unsaturated monocyclic ring. In some embodiments, one or more -Cy- is independently a bivalent optionally substituted aromatic monocyclic ring.

In some embodiments, one or more -Cy- is independently an optionally substituted 6-10 membered arylene. In some embodiments, one or more -Cy- is independently an optionally substituted a 5-10 membered heteroarylene having 1-4 heteroatoms independently selected from oxygen, nitrogen, or sulfur. In some embodiments, one or more -Cy- is independently an optionally substituted a 5-6 membered heteroarylene having 1-4 heteroatoms independently selected from oxygen, nitrogen, or sulfur. In some embodiments, one or more -Cy- is independently an optionally substituted 5 membered heteroarylene having 1-4 heteroatoms independently selected from oxygen, nitrogen, or sulfur. In some embodiments, one or more -Cy- is independently an optionally substituted a 6 membered heteroarylene having 1-4 heteroatoms independently selected from oxygen, nitrogen, or sulfur.

Exemplary optionally substituted -Cy-heteroarylene groups include thienylene, furanylene, pyrrolylene, imidazolylene, pyrazolylene, triazolylene, tetrazolylene, oxazolylene, isoxazolylene, oxadiazolylene, thiazolylene, isothiazolylene, thiadiazolylene, pyridylene, pyridazinylene, pyrimidinylene, pyrazinylene, indolizinylene, purinylene, naphthyridinylene, pteridinylene, indolylene, isoindolylene, benzothienylene, benzofuranylene, dibenzofuranylene, indazolylene, benzimidazolylene, benzthiazolylene, quinolylene, isoquinolylene, cinnolinylene, phthalazinylene, quinazolinylene, quinoxalinylene, 4H-quinolizinylene, carbazolylene, acridinylene, phenazinylene, phenothiazinylene, phenoxazinylene, tetrahydroquinolinylene, tetrahydroisoquinolinylene, pyrido[2,3-b]-1,4-oxazin-3(4H)-onylene, and chromanylene.

In certain embodiments, -Cy- is selected from the group consisting of tetrahydropyranylene, tetrahydrofuranylene, morpholinylene, thiomorpholinylene, piperidinylene, piperazinylene, pyrrolidinylene, tetrahydrothiophenylene, and tetrahydrothiopyranylene, wherein each ring is optionally substituted.

In some embodiments, one or more -Cy- is independently an optionally substituted 3-8 membered carbocyclylene. In some embodiments, one or more -Cy- is independently an optionally substituted 3-6 membered carbocyclylene. In some embodiments, one or more -Cy- is independently an optionally substituted cyclopropylene, cyclobutylene, cyclopentylene, or cyclohexylene. In some embodiments, one or more -Cy- is independently an optionally substituted cyclobutylene.

In some embodiments, one or more -Cy- is independently an optionally substituted 3-10 membered heterocyclylene having 1-4 heteroatoms independently selected from oxygen, nitrogen, or sulfur. In some embodiments, one or more -Cy- is independently an optionally substituted 3-8 membered heterocyclylene having 1-4 heteroatoms independently selected from oxygen, nitrogen, or sulfur. In some embodiments, one or more -Cy- is independently an optionally substituted 5-7 membered heterocyclylene having 1-3 heteroatoms independently selected from oxygen, nitrogen, or sulfur. In some embodiments, one or more -Cy- is independently an optionally substituted 3 membered heterocyclylene having 1 heteroatom independently selected from oxygen, nitrogen, or sulfur. In some embodiments, one or more -Cy- is independently an optionally substituted 4 membered heterocyclylene having 1 heteroatom independently selected from oxygen, nitrogen, or sulfur. In some embodiments, one or more -Cy- is independently an optionally substituted 5 membered heterocyclylene having 1-2 heteroatoms independently selected from oxygen, nitrogen, or sulfur. In some embodiments, one or more -Cy- is independently an optionally substituted 6 membered heterocyclylene having 1-3 heteroatoms independently selected from oxygen, nitrogen, or sulfur.

In some embodiments, one or more -Cy- is independently an optionally substituted partially unsaturated 4-10 membered heterocyclylene having 1-4 heteroatoms independently selected from oxygen, nitrogen, or sulfur. In some embodiments, one or more -Cy- is independently an optionally substituted partially unsaturated 5-7 membered heterocyclylene having 1-3 heteroatoms independently selected from oxygen, nitrogen, or sulfur. In some embodiments, one or more -Cy- is independently an optionally substituted partially unsaturated 5 membered heterocyclylene having 1-2 heteroatoms independently selected from oxygen, nitrogen, or sulfur. In some embodiments, one or more -Cy- is independently an optionally substituted partially unsaturated 6 membered heterocyclylene having 1-3 heteroatoms independently selected from oxygen, nitrogen, or sulfur.

Exemplary -Cy- partially unsaturated 5 membered optionally substituted heterocyclylenes include dihydroimidazolylene, dihydrooxazolylene, dihydrothiazolylene, dihydrothiadiazolylene, and dihydrooxadiazolylene.

Exemplary -Cy- saturated 3-8 membered optionally substituted heterocyclenes include oxiranylene, oxetanylene, tetrahydrofuranylene, tetrahydropyranylene, oxepaneylene, aziridineylene, azetidineylene, pyrrolidinylene, piperidinylene, azepanylene, thiiranylene, thietanylene, tetrahydrothiophenylene, tetrahydrothiopyranylene, thiepanylene, dioxolanylene, oxathiolanylene, oxazolidinylene, imidazolidinylene, thiazolidinylene, dithiolanylene, dioxanylene, morpholinylene, oxathianylene, piperazinylene, thiomorpholinylene, dithianylene, dioxepanylene, oxazepanylene, oxathiepanylene, dithiepanylene, diazepanylene, dihydrofuranonylene, tetrahydropyranonylene, oxepanonylene, pyrrolidinonylene, piperidinonylene, azepanonylene, dihydrothiophenonylene, tetrahydrothiopyranonylene, thiepanonylene, oxazolidinonylene, oxazinanonylene, oxazepanonylene, dioxolanonylene, dioxanonylene, dioxepanonylene, oxathiolinonylene, oxathianonylene, oxathiepanonylene, thiazolidinonylene, thiazinanonylene, thiazepanonylene, imidazolidinonylene, tetrahydropyrimidinonylene, diazepanonylene, imidazolidinedionylene, oxazolidinedionylene, thiazolidinedionylene, dioxolanedionylene, oxathiolanedionylene, piperazinedionylene, morpholinedionylene, and thiomorpholinedionylene.

In certain embodiments, one or more -Cy- is independently an optionally substituted azetidineylene.

In certain embodiments, one or more -Cy- is independently an optionally substituted pyrrolidinylene.

In certain embodiments, one or more -Cy- is independently an optionally substituted piperidinylene.

In certain embodiments, one or more -Cy- is independently an optionally substituted homopiperidinylene.

In some embodiments, L is of any one of the following formulae:

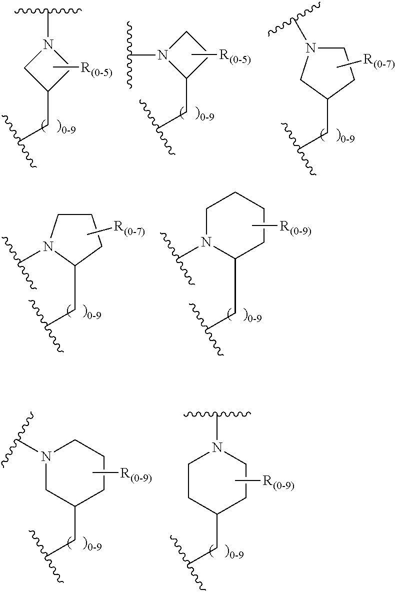

wherein each R is independently as defined and described above and herein.

In some embodiments, L is of either of the following formulae:

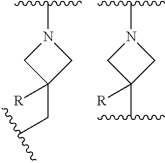

wherein each R is independently as defined and described above and herein.

In some embodiments, L is of any one of the following structures:

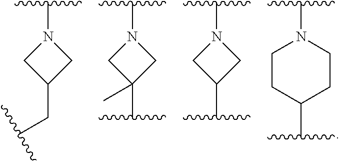

In some embodiments, R² is R.

In some embodiments, R² is halogen.

In some embodiments, R² is an optionally substituted C₁₋₆ heteroaliphatic group.

In some embodiments, R² is an optionally substituted C₁₋₆ aliphatic group.

In certain embodiments, R² is optionally substituted methyl. In some embodiments, R² is optionally substituted ethyl. In some embodiments, R² is optionally substituted isopropyl. In some embodiments, R² is optionally substituted neopentyl. In some embodiments, R² is optionally substituted cyclobutyl. In some embodiments, R² is an optionally substituted oxetane.

Exemplary optionally substituted R² groups are as depicted below:

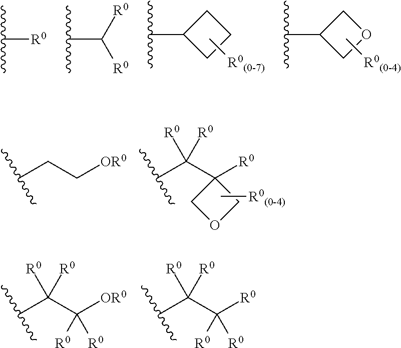

wherein each R^{o} is independently as defined and described above and herein.

Exemplary R² groups are as depicted below:

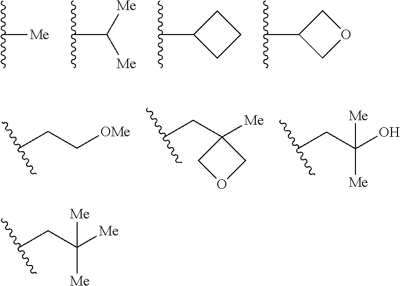

In some embodiments, the present invention provides a method for preparing a compound of any of the following formulae:

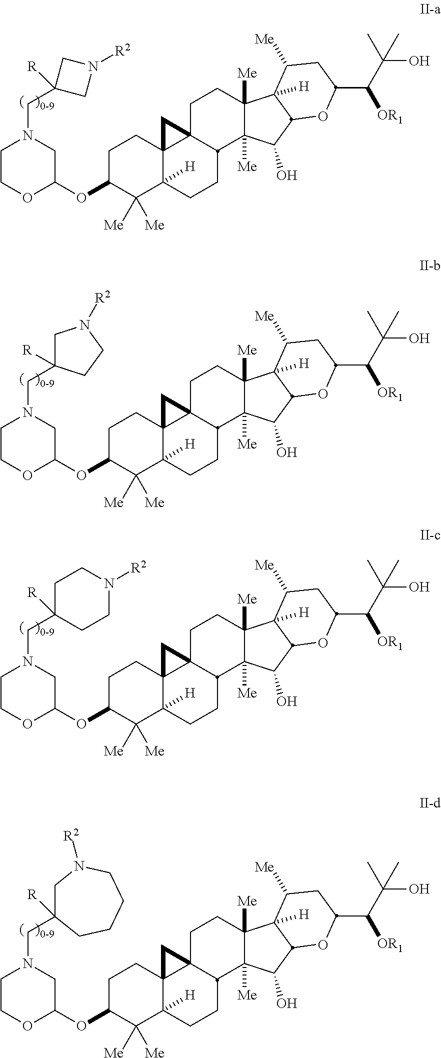

wherein each of R¹, R², and R is independently as defined and described above and herein.

In some embodiments, the present invention provides a method for preparing a compound of any of the following formulae:

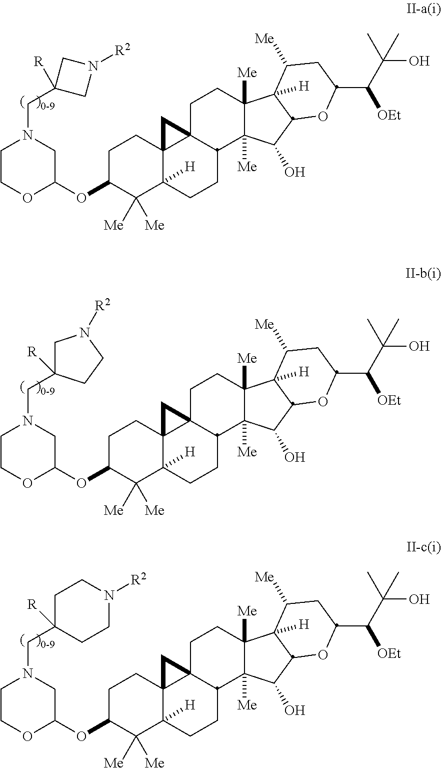

wherein each of R² and R is independently as defined and described above and herein.

In some embodiments, the present invention provides a method for preparing a compound of any of the following formulae:

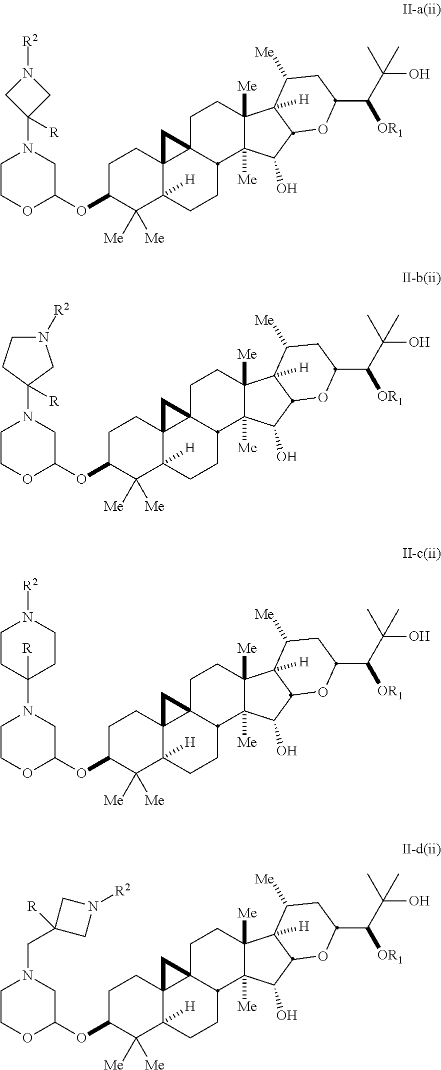

wherein each of R¹, R², and R is independently as defined and described above and herein.

In some embodiments, the present invention provides a method for preparing a compound of any of the following formulae:

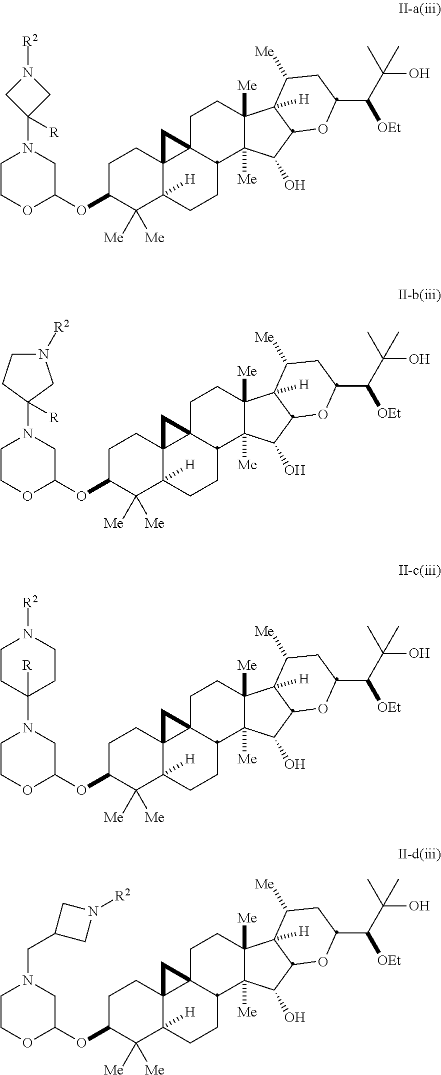

wherein each of R² and R is independently as defined and described above and herein.

In some embodiments, the present invention provides a method for preparing a compound of the formula:

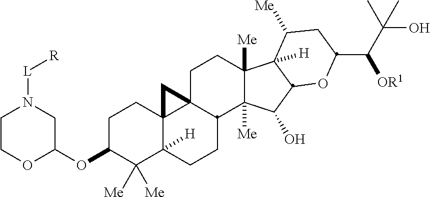

wherein each of R, L, and R¹ is independently as defined and described above and herein.

In some embodiments, the present invention provides a method for preparing a compound of any of the following formulae:

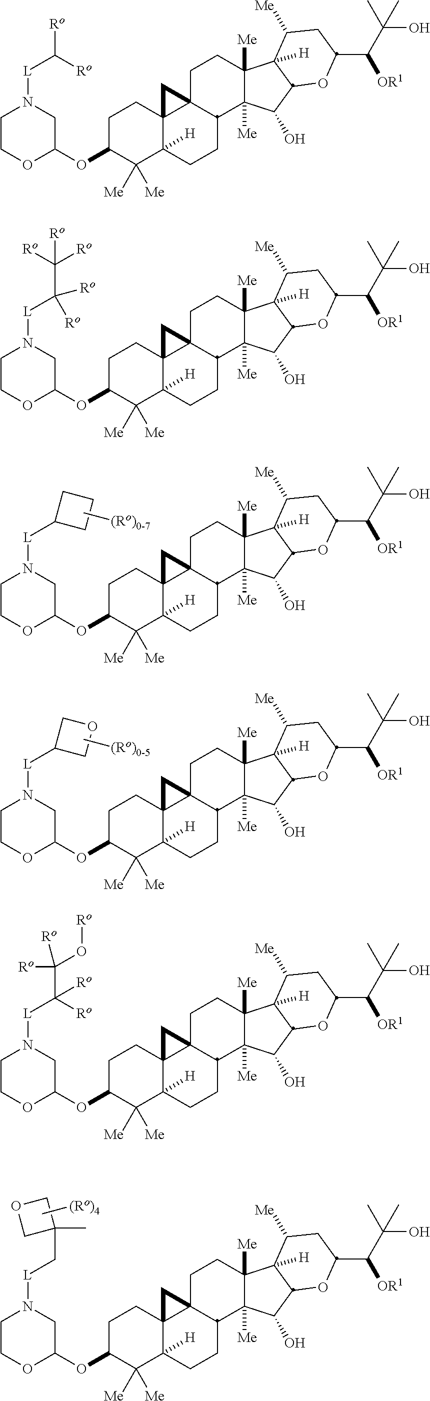

wherein each of L, R^{o}, and R¹ is independently as defined and described above and herein. In some embodiments, a compound is as depicted above, wherein R¹ is R. In certain embodiments, a compound is as depicted above, wherein R¹ is ethyl.

In some embodiments, the present invention provides a method for preparing a compound of any of the following formulae:

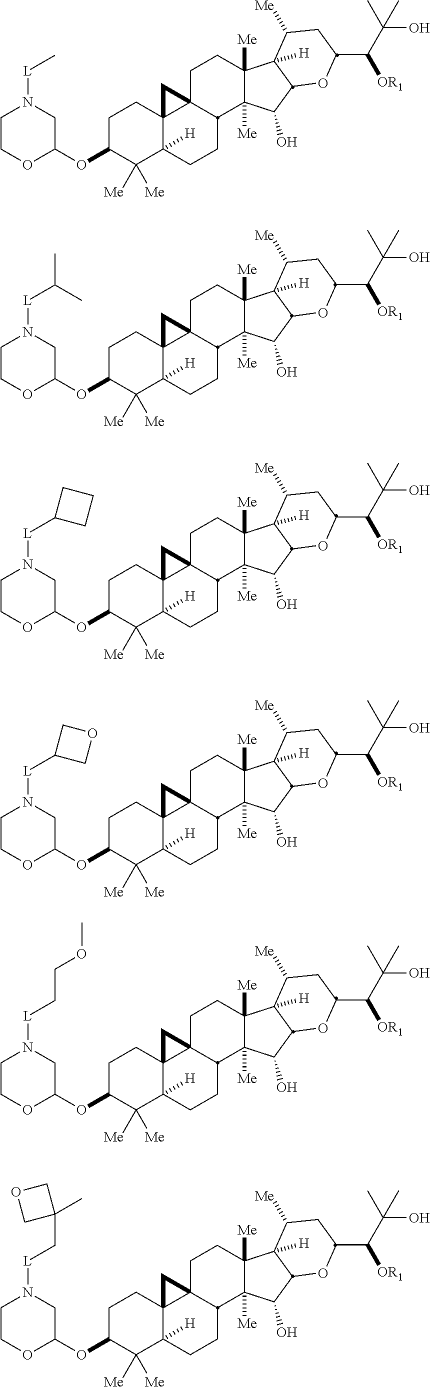

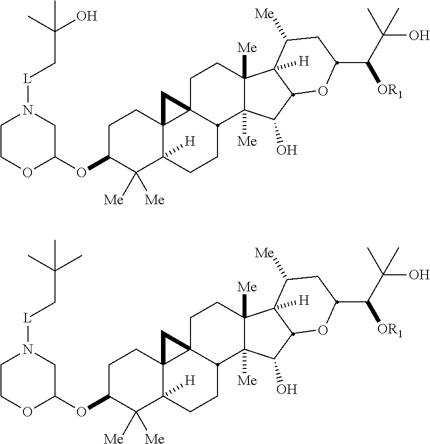

wherein each of L and R¹ is independently as defined and described above and herein. In some embodiments, a compound is as depicted above, wherein R¹ is R. In certain embodiments, a compound is as depicted above, wherein R¹ is ethyl.

In some embodiments, the present invention provides a method for preparing a compound of any of the following structures in Table 1:

TABLE 1

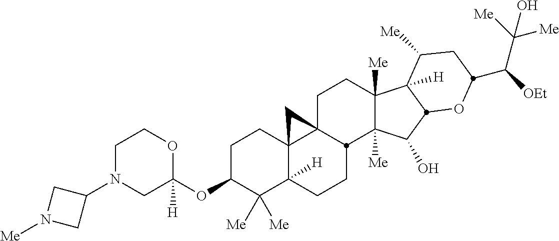

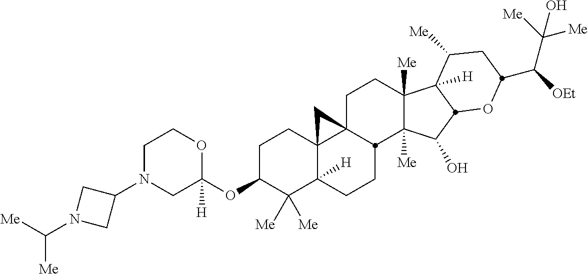

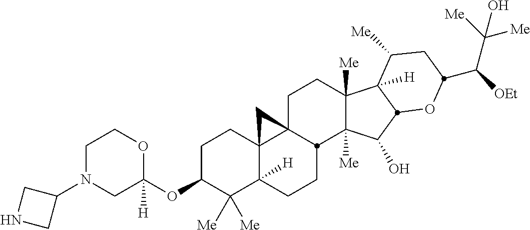

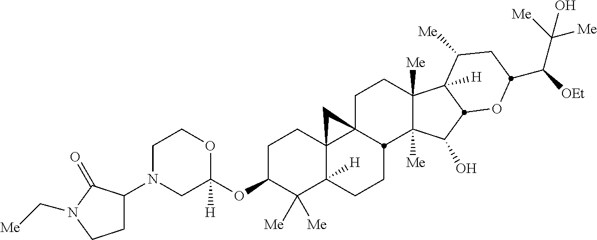

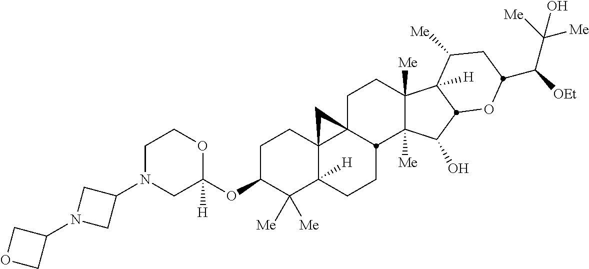

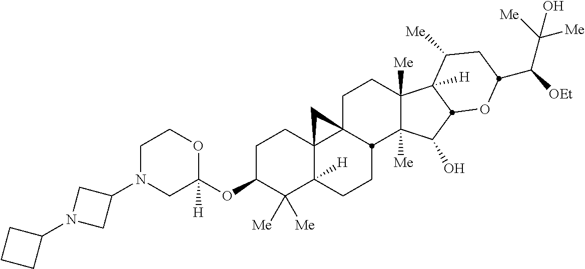

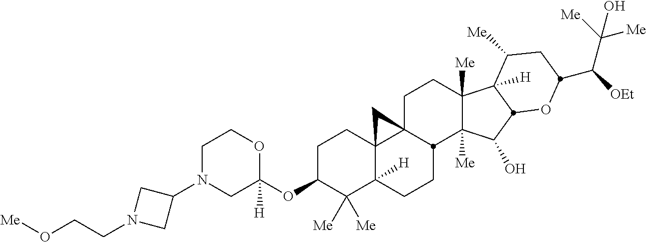

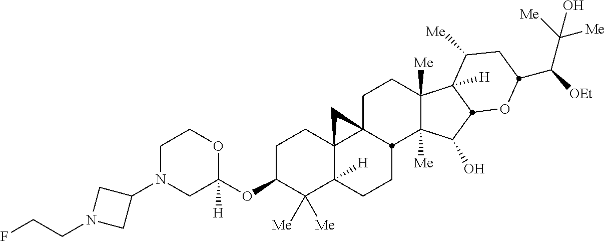

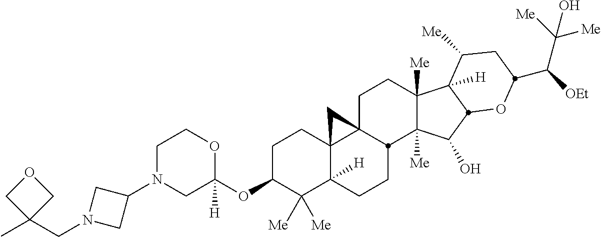

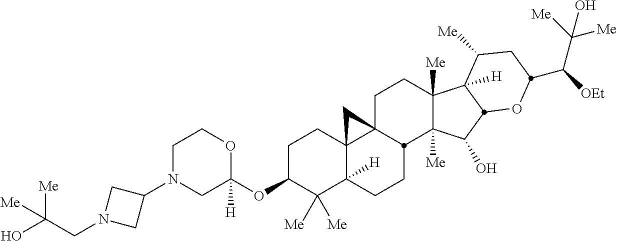

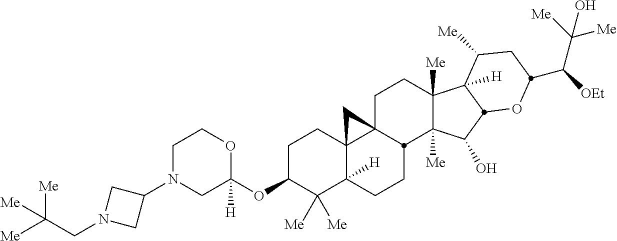

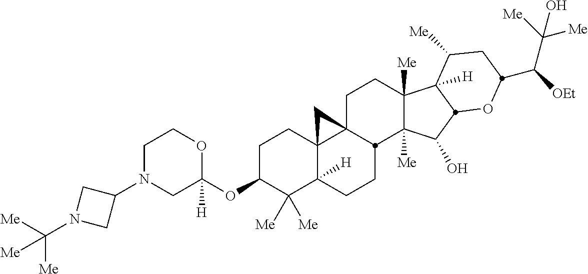

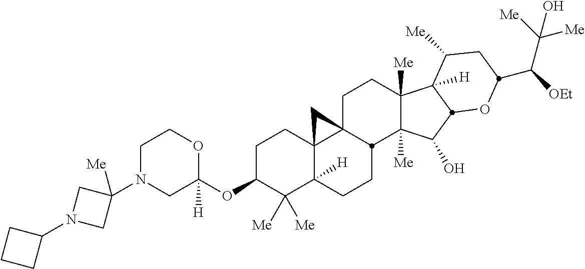

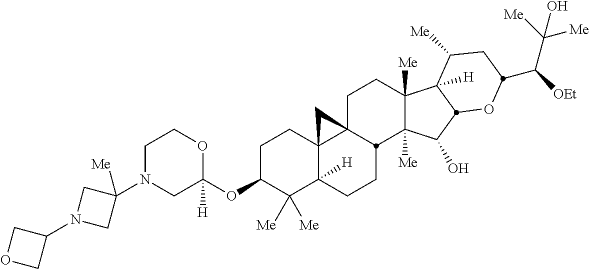

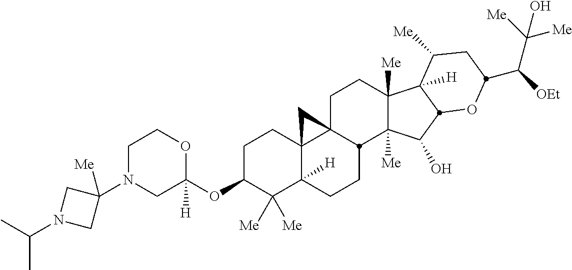

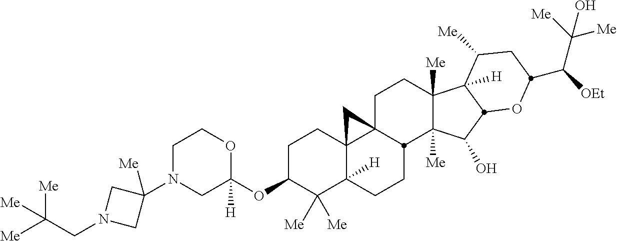

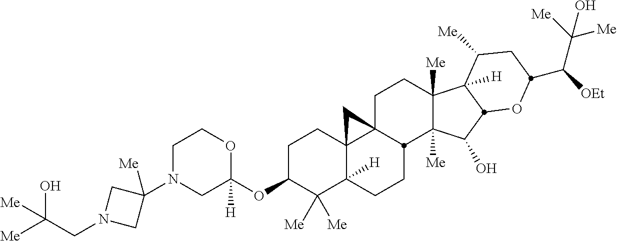

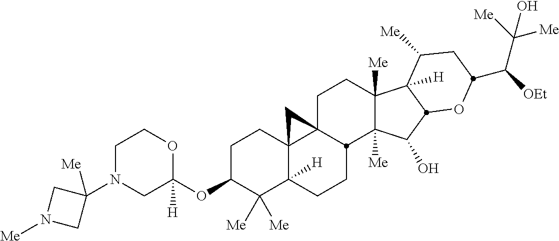

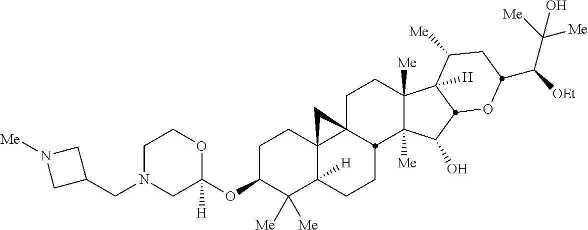

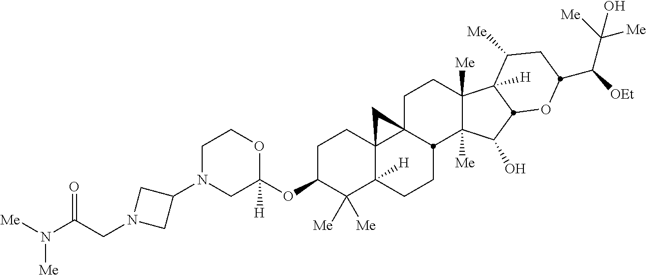

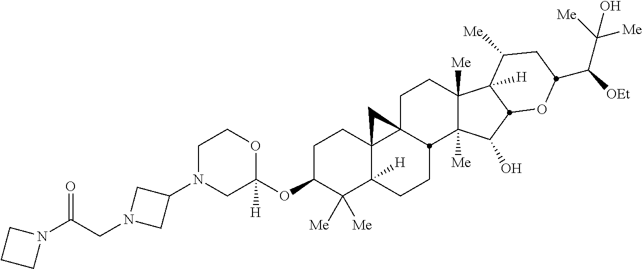

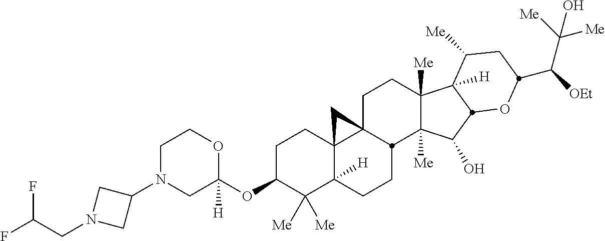

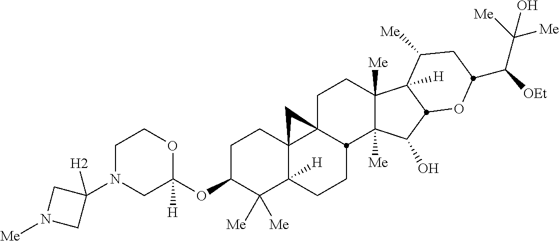

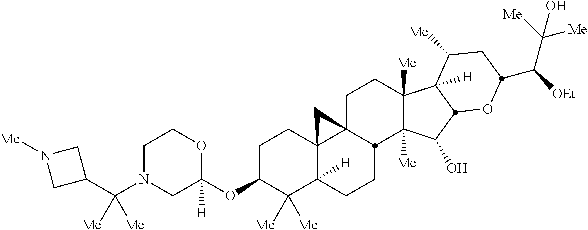

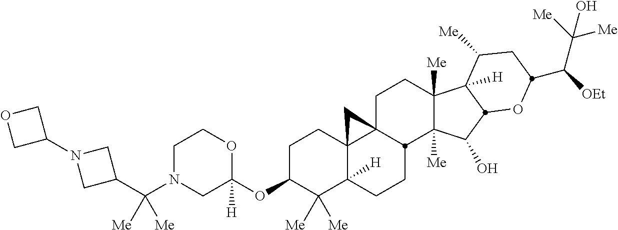

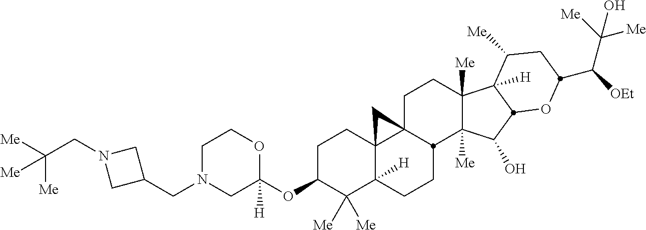

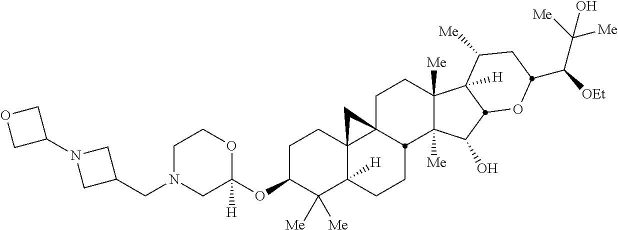

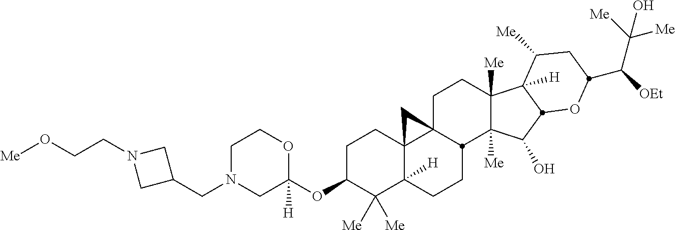

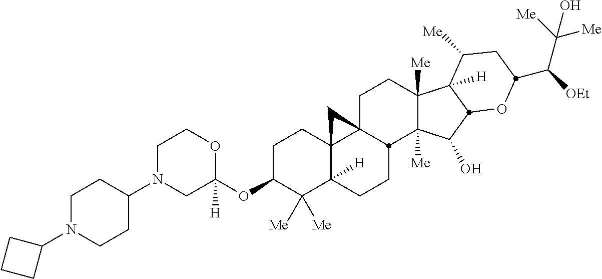

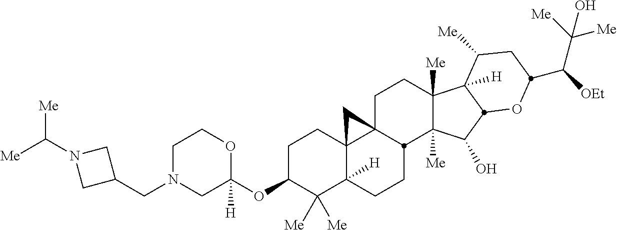

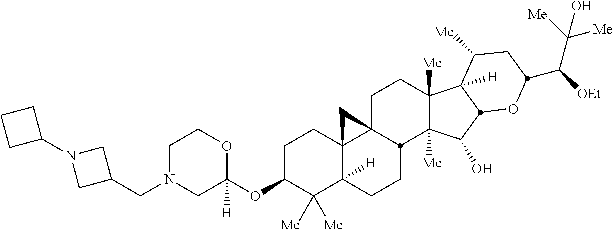

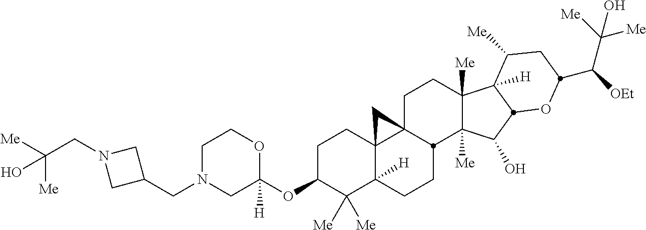

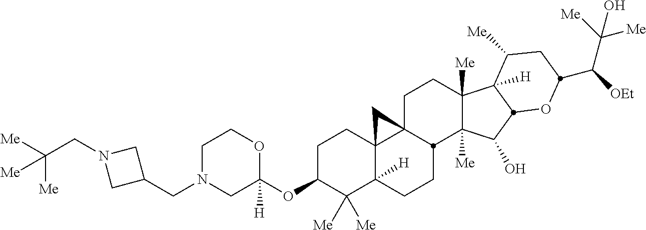

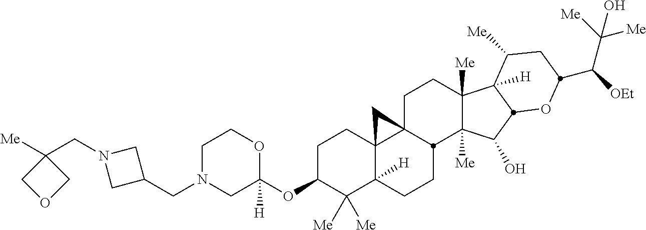

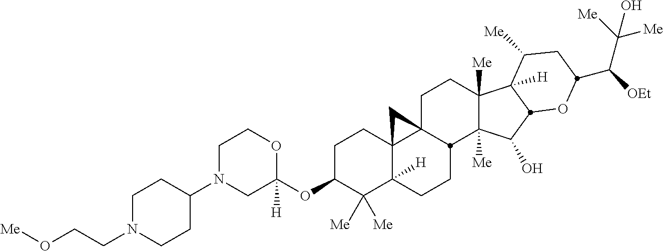

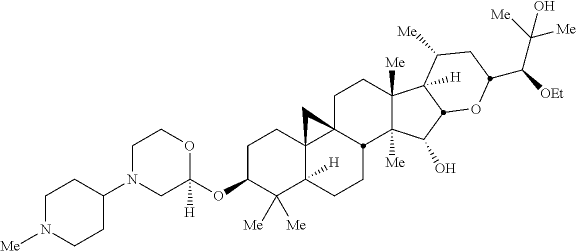

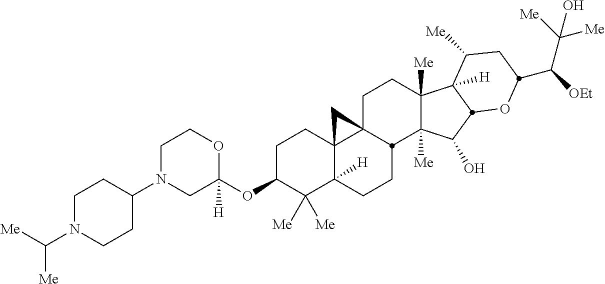

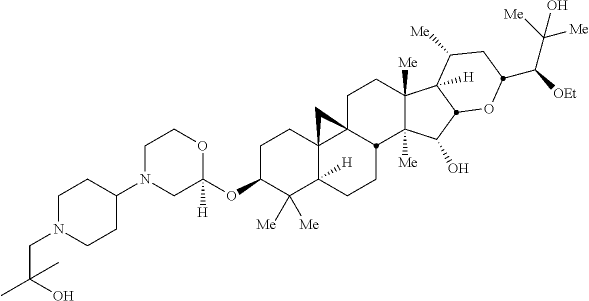

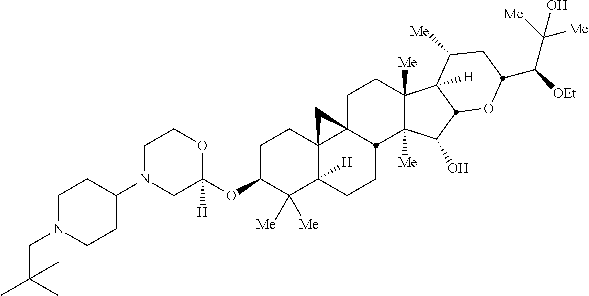

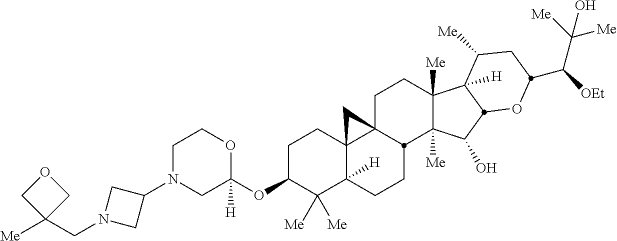

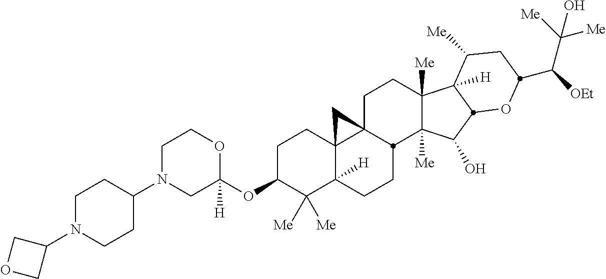

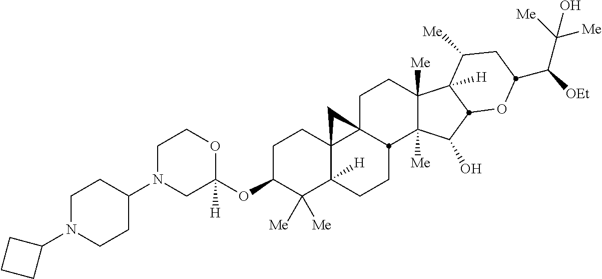

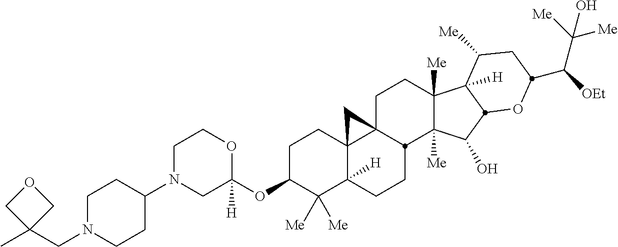

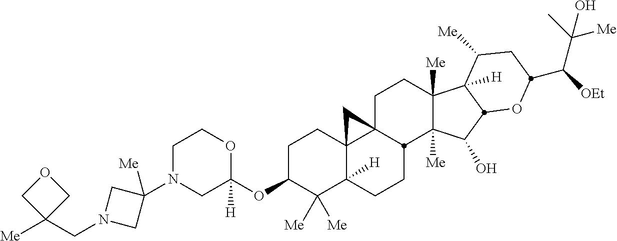

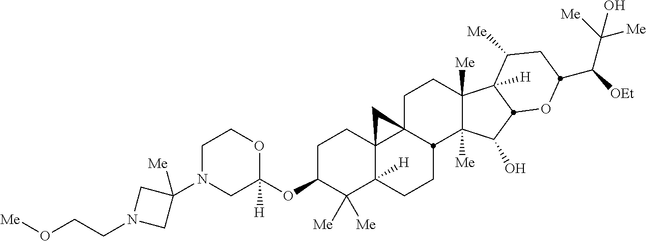

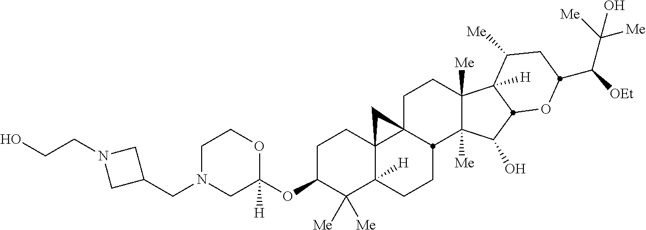

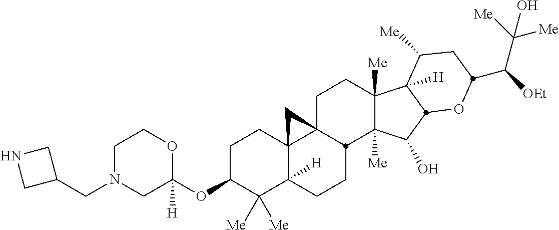

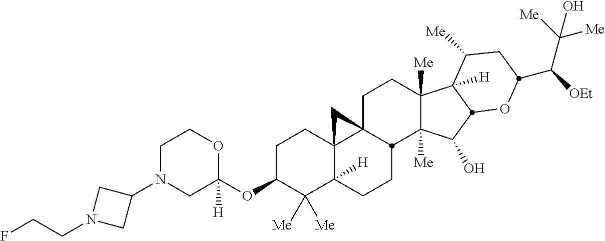

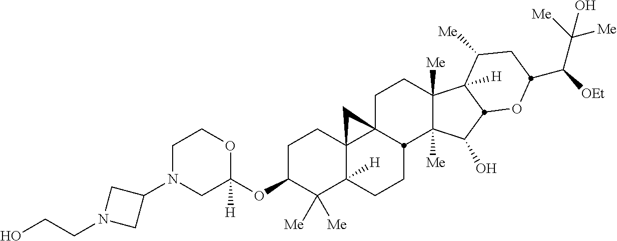

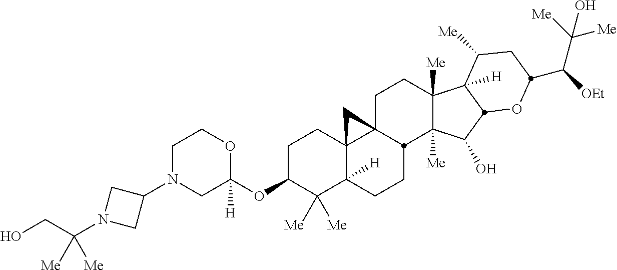

Various functions and advantages of these and other embodiments of the present invention will be more fully understood from the examples described below. The following examples are intended to illustrate the benefits of the present invention, but do not exemplify the full scope of the invention.

### Exemplification

The following experimentals describe the isolation of compounds for use in methods of the present invention. Melting points are uncorrected. ¹H and ¹³C NMR spectra were measured at 400 and 100 MHz respectively in CDCl₃ or pyridine-d5. Chemical shifts are downfield from trimethylsilane (TMS) as internal standards, and J values are in hertz. Mass spectra were obtained on API-2000, or Hewlett Parkard series 1100 MSD with ESI technique. All solvents used were reagent grade. The black cohosh extract was obtained from Hauser Pharmaceuticals, Avoca Inc and Indena SpA. This extract is substantially equivalent to the USP preparation of black cohosh extract, in which about 50% aqueous ethanol is used to extract powdered root and then concentrated to near dryness. Other abbreviations include: Ac₂O (acetic anhydride), DMAP (dimethylaminopyridine), PhI(OAc)₂ (iodosobenzene diacetate), PDC (pyridinium dichromate), TFAA (trifluoroacetic acid), DMDO (dimethyldioxirane), DIPEA (N,N-Diisopropylethylamine), RB (round-bottom), TLC (thin layer chromatography), MeOH (methanol), MeOD (methanol d-4), /-PrOH (isopropanol), TBDMS (tert-butyldimethylsilyl-), TBS (tert-butyldimethylsilyl-), DHEA (dehydroepiandrosterone), TBHP (tert-butylhydroperoxide), DMSO (dimethylsulfoxide), KOt-Bu (potassium tert-butoxide), MS (mass spectrometry), Mom-Cl (Chloromethyl methyl ether), EtOAc (ethyl acetate), M.P. (melting point), EtPPh₃I (ethyltriphenylphosphonium iodide), Et₃N (triethyl amine), mCPBA (met[alpha]-chloroperbenzoic acid), BF₃OEt₂ (trifluoroborane etherate), EtOH (ethanol), HPLC (high performance liquid chromatography), LCMS (liquid chromatography mass spectrometry), NMR (nuclear magnetic resonance).

General procedures: Reagents were acquired commercially and used without further purification except where noted. LC/MS spectra were acquired using an Agilent MSD with electrospray ionization and Agilent 1100 series LC with a Zorbax C-18 column (2.1×30 mm, 3.5 micron particle size). Standard LC conditions utilized CH₃CN with 0.1% formic acid as the organic phase and water containing 0.1% formic acid as the aqueous phase, and were run as follows: Flow rate 1.000 mL/min; 0-1.80 minutes 2-98% organic-aqueous; 1.80-3.75 minutes 98% organic-aqueous, 3.75-3.76 minutes 98-2% organic-aqueous; 3.76-4.25 minutes 2% organic-aqueous. LC/MS samples included here are of reaction mixtures pre-workup unless otherwise noted. Automatic integration over the entire non-background signal is included here, and selected key masses for individual regions have been added manually. NMR spectra were acquired using a Varian 400 MHz instrument and are acquired in CDCl₃.

### Example 1

The black cohosh extract, utilized in the protocol described below, was obtained using the following extraction protocol.

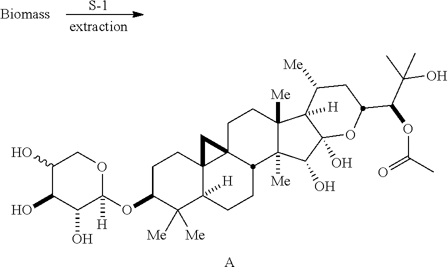

The black cohosh biomass was first dried and ground to a suitable particle size usually ranging from about 0.1 to about 1.0 mm³. This may be accomplished by passage through a chipper or a grinding mill. The ground biomass (1.88 kg) was extracted with tech grade methanol (9.4 L) at 50° C. for 2 hours. It should be noted that the ground biomass can alternatively be extracted using other alcohols, for instance 95% ethanol, and that the extraction can take place at ambient temperatures for about 22 hours. The extract solution was filtered through Celite using a basket centrifuge. The filter cake was rinsed with tech grade methanol and the filtrate were combined. The clear, homogeneous, dilute methanol extract was concentrated under vacuum with a maximum temperature 33° C. reached, which provided 1.3 L of concentrated solution in which suspended solids were visible. The concentrated extract was added slowly to 5% KCl solution in water (5.2 L) and the resulting mixture was cooled to 4° C. and held for 2 hours. Other salts can also be used, including but not limited to, (NH₄)₂SO₄, K₂SO₄, NaCl, etc. The concentration of salt in water can range from about 3% to about 30%. The holding time can range from about 2 hours to about 24 hours. The precipitate containing compound A was formed, which was collected using a centrifuge and rinsed with water. An aqueous salt solution can also be used to rinse the solid, including but not limited to, about 0-30% (NH₄)₂SO₄, K₂SO₄, KCl, NaCl, etc. In some instances, Celite was added as filter aid to facilitate the filtration. The collected solids were transferred to a dryer (e.g., a spray dryer, drum dryer, etc], which provided 71 g of dry solid.

The above solid was taken up in 210 mL of CH₂Cl₂ and the obtained slurry was stirred at RT for 1 h, followed by addition of 268 mL of 10% NaCl. The organic phase was collected and the aqueous layer was extracted again with 70 mL of CH₂Cl₂. The combined organic phase was evaporated to dryness, which afforded 56.7 g of solid, which contains 13% of A by HPLC-ELSD analysis.

HPLC analysis conditions:

Column: Phenomenex Luna C18(2), 3 μm, 4.6 mm×150 mm
Flow rate: 1.0 mL/min

### Detector: ELSD, Temp.: 55° C., Gain 11

| Time | water (v/v %) | acetonitrile (v/v %) | methanol (v/v %) |
|---|---|---|---|
| 0.0 | 40 | 35 | 25 |
| 10.0 | 25 | 50 | 25 |
| 15.0 | 5 | 70 | 25 |
| 18.0 | 5 | 70 | 25 |
| 18.1 | 40 | 35 | 25 |
| 23.0 | 40 | 35 | 25 |

Rt of A1 (xyloside)=7.9 min
Rt of A2 (arabinoside)=7.2 min

### Step S-2

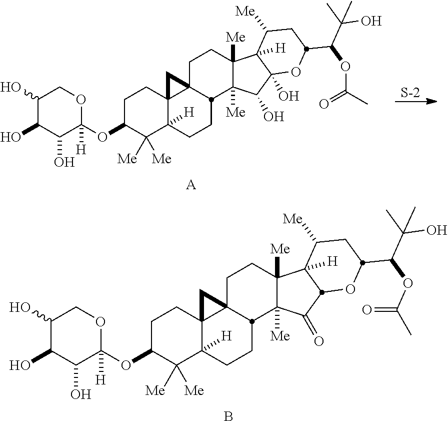

### Method A:

To a solution of the solid obtained from S-1 (20.3 g, 13% A) in CH₂Cl₂ (162 mL) was added ZrCl₄ (1.32 g) at 20° C. in three portions over 1 h. The mixture was stirred at 20° C. for an additional 35 minutes and Celite (7.1 g) was added, followed by addition of Et₃N (5 mL) within 5-15 minutes. The solids were filtered off and washed with CH₂Cl₂ (100 mL). The filtrates were combined and washed with half saturated NaHCO₃ (100 mL). The aqueous layer was back extracted with CH₂Cl₂ (25 mL). All the organic layers were combined and evaporated to dryness, which afforded crude product B (19.16 g). Purification of the crude on SiO₂ (100 g) with 0-7% MeOH/CH₂Cl₂ provided B (4.07 g) in 58% purity based on HPLC-ELSD analysis. Precipitation of the solid in EtOH/water (41 mL/49 mL) at 5° C. provided an upgraded compound B (2.4 g) in 83.3% purity by HPLC-ELSD analysis.

HPLC-ELSD conditions: see above
Rₜ of B1 (xyloside)=7.2 min
Rₜ of B2 (arabinoside)=6.7 min

Method B:

Alternatively, the solid obtained from S-1 (32 g, 13% A) was dissolved in DMSO (70 mL), filtered through Celite and purified by reverse phase chromatography with C-18 column (40-63 μm, 18.2 cm×45 cm) using 60-70% MeOH/water as eluents. The fractions were analyzed using the analytical HPLC conditions described above. The selected fractions were combined and concentrated to about half of the original volume (1.1 L). NaCl (143 g) was added and the resulting mixture was extracted with CH₂Cl₂ (2×340 mL). The combined organic phase was concentrated to dryness. Further drying in vacuo provided 4.0 g of solid A in 62.3% purity by HPLC-ELSD analysis.

### Step S-2

To a solution of the above solid (62.3% A, 4.0 g) in CH₂Cl₂ (80 mL) was added ZrCl₄ (200 mg) at 20° C. The mixture was stirred at 20° C. for 75 min and Celite (4.0 g) was added followed by addition of Et₃N (0.83 mL) within 5-15 min. The solids were filtered off and washed with CH₂Cl₂ (51 mL). The filtrates were combined and most solvent was removed by distillation at 30-40° C. The residue was azeotroped with EtOH to remove the rest of CH₂Cl₂. Precipitation of the residue in EtOH/H₂O (9/11) provided compound B (1.2 g) in 96% purity by HPLC-ELSD analysis. HPLC-ELSD conditions: see above. Rₜ of B-i(xyloside)=7.2 min; Rₜ of B-ii(arabinoside)=6.7 min.

### Step S-3/Step S-4:

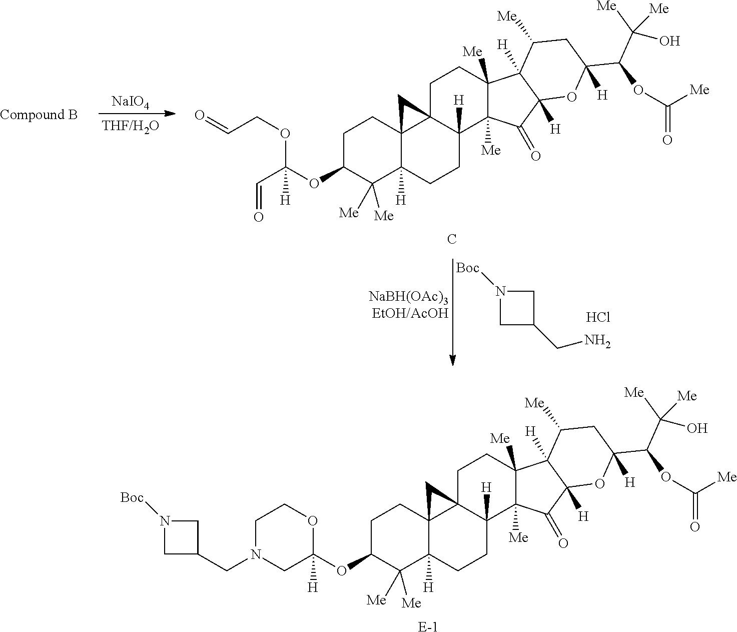

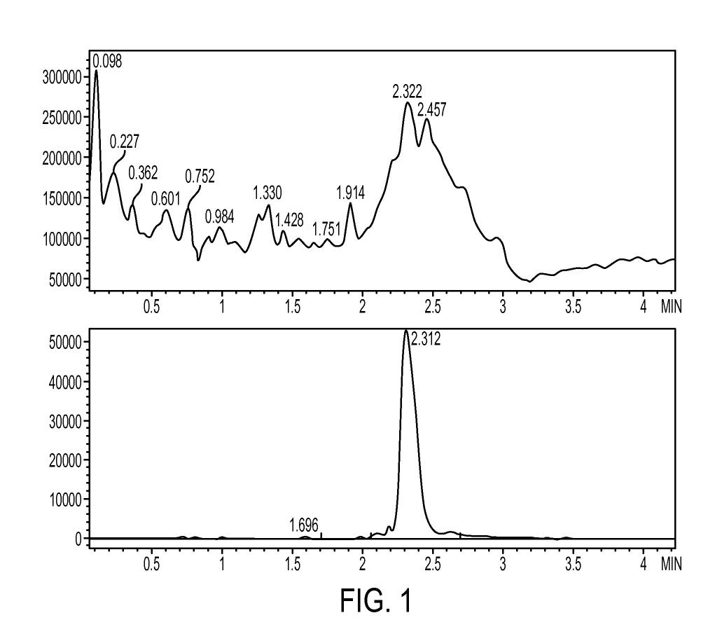

In a 1-L round-bottomed flask, Compound B (50 g, 75.4 mmol) was dissolved in THF (600 mL) and H₂O (200 mL), treated with NaIO₄ (64.4 g, 301.7 mmol), and the resulting mixture was heated to 50° C. and stirred vigorously (>1000 rpm) for 17 h. The reaction progress was followed by LC/MS until no more mono-oxidative cleavage product [M+1, 661] was observed, then was cooled to RT and THF was removed in vacuo. The residue was diluted with CH₂Cl₂ (300 mL) and H₂O (300 mL) and stirred at RT for 30 min. The mixture was then partitioned between CH₂Cl₂ (800 mL) and H₂O (800 mL). A solution of aq. HCl (1.0 M, 300 mL) was added and the layers were separated. The aqueous layer was extracted with CH₂Cl₂ (1 L, 2×500 mL), and the combined organic layers were washed with 10% NaOAc (300 mL), dried over Na₂SO₄, filtered, and concentrated in vacuo. The dialdehyde compound C was obtained as a crude yellow solid (51.5 g) and was carried on to the next step without further purification, assuming quantitative yield. (FIG. 1)

To a solution of 1-Boc-3-(aminomethyl)-azetidine (15.5 g, 82.9 mmol) in EtOH (250 mL) was slowly added aq. HCl (1.0 M, 83 mL). The solvent was removed in vacuo at 38° C., providing the hydrochloride salt of the amine as a white solid (18.0 g).

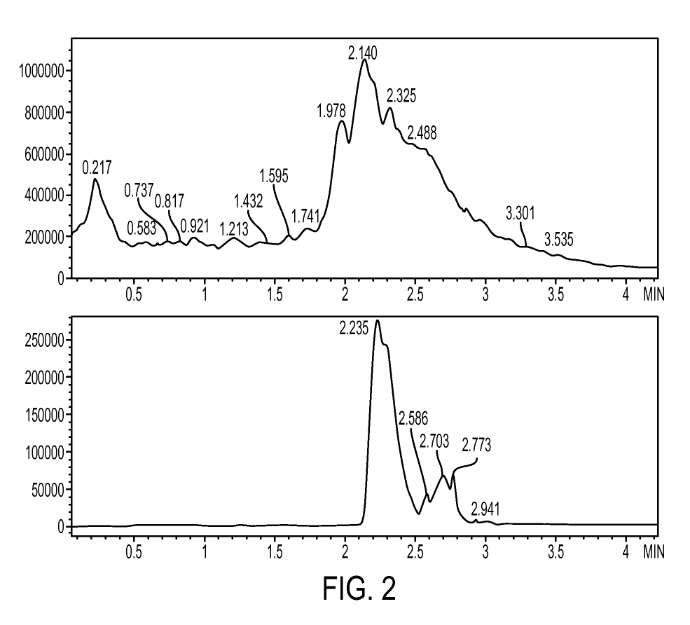

A solution of dialdehyde C (75.4 mmol) in absolute EtOH (450 mL) was treated with 1-Boc-3-(aminomethyl)-azetidine hydrochloride (18.0 g, 82.9 mmol) and AcOH (50 mL). The reaction mixture was stirred at RT for 10 min, then NaBH(OAc)₃ (48 g, 226 mmol) was added. The reaction was stirred at RT and monitored by LC/MS. After 1 h the starting material was completely consumed, and the major product observed was the desired morpholine E-1 (m/z M+1, 785). The reaction mixture was then partitioned between CH₂Cl₂ (750 mL) and H₂O (750 mL), and the organic layer was collected. The aqueous layer was extracted with CH₂Cl₂ (500 mL, 400 mL), and the combined organic layers were dried over Na₂SO₄, filtered, and concentrated in vacuo. The residue was azeotroped with toluene to completely remove AcOH and dried under high vacuum to provide E-1 as a yellow powder (64 g), which was carried on to the next step assuming quantitative yield. (FIG. 2)

### Step S-5

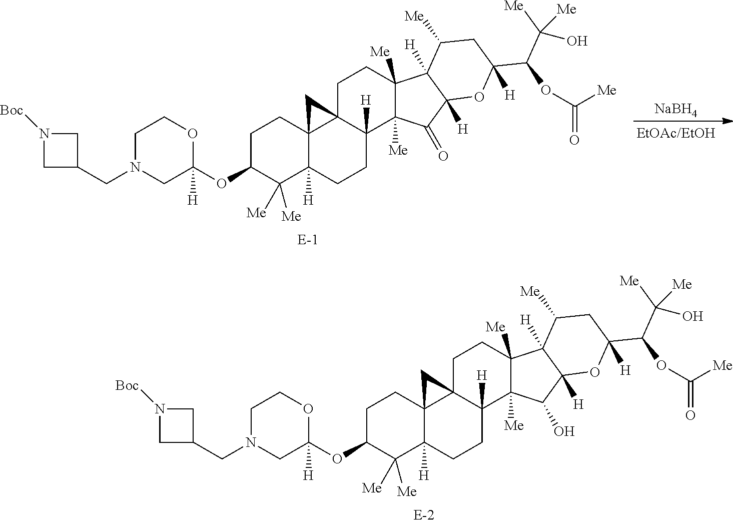

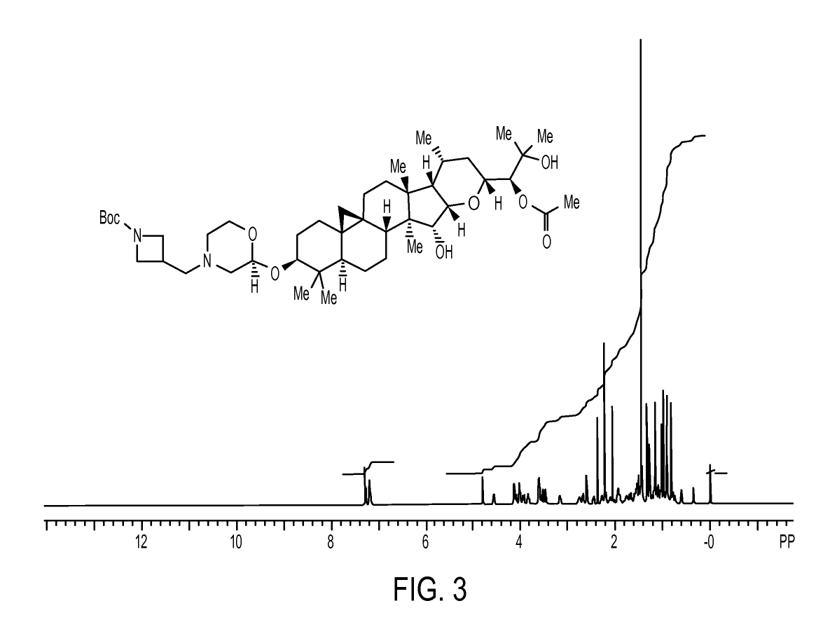

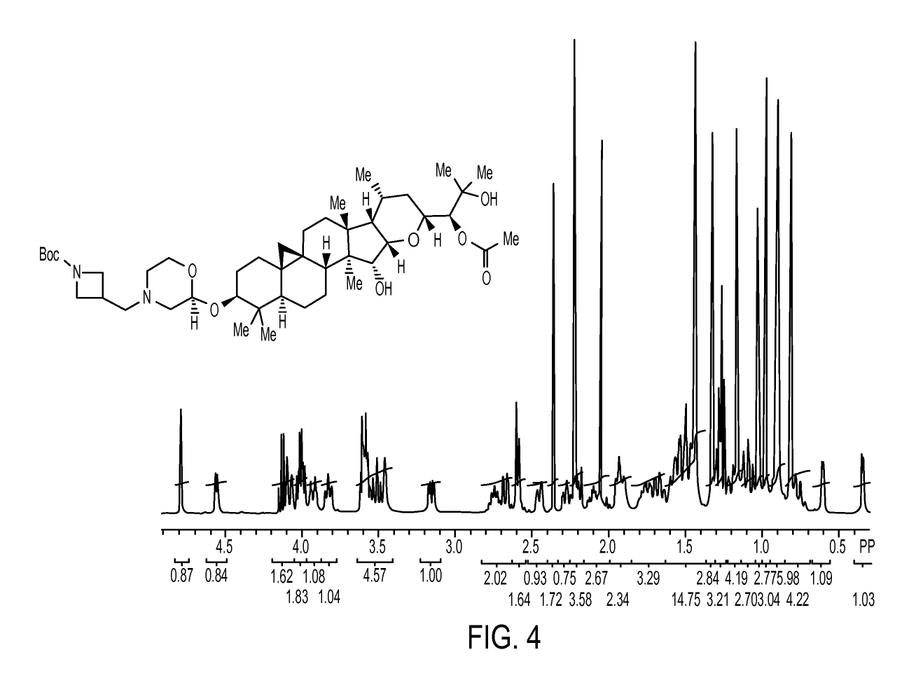

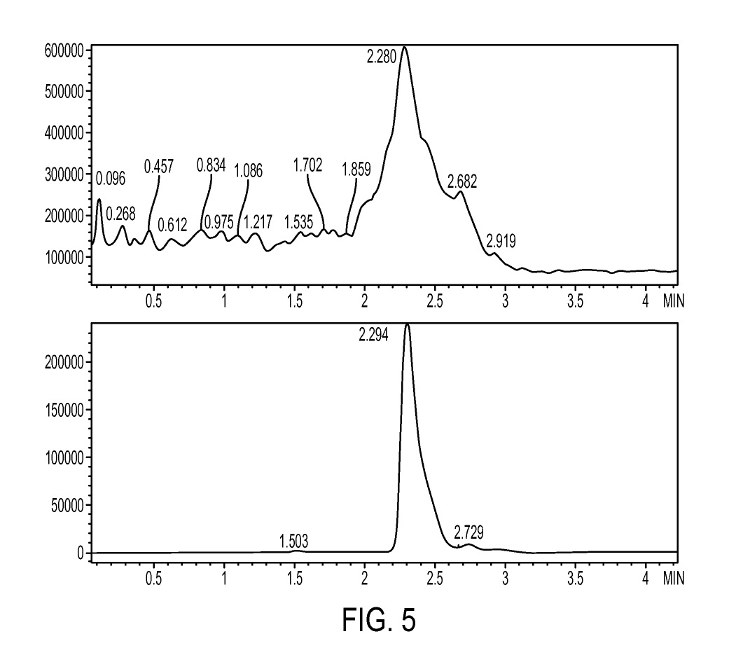

In a 1-L round bottom flask, a slurry of NaBH₄ (3766 mg, 99.6 mmol) in absolute EtOH (60 mL) was stirred at RT for 10 min. A solution of ketone E-1 (90.5 mmol) in EtOAc (600 mL) was added over 3 min. The reaction was stirred at RT and monitored by LC/MS. After 30 min the starting material was completely consumed, and the major product observed was the desired alcohol E-2 (m/z M+1, 787). The reaction was carefully quenched with AcOH (17.1 mL, 0.3 mol, 3.3 eq) (Caution: gas evolution), and then was partitioned between CH₂Cl₂ (650 mL) and H₂O (650 mL). The layers were separated and the aqueous layer was extracted with CH₂Cl₂ (400 mL, 300 mL). The combined organic layers were dried over Na₂SO₄, filtered, and concentrated in vacuo. The residue was azeotroped with toluene to completely remove AcOH and dried under high vacuum to provide a crude yellow powder. Purification of the residue on a 750 G silica gel column with 50-100% EtOAc/hexane provided compound E-2 as a light yellow solid (22.4 g, 31% yield over 3 steps). (FIGS. 3-5)

### Step S-6

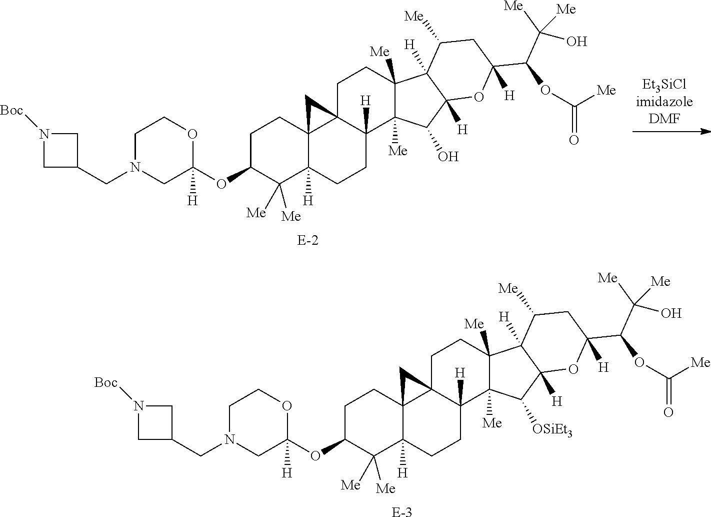

Note: The amount of Et₃SiCl needed for this reaction is variable depending on the purity of compound E-2. In some instances, excess amounts of Et₃SiCl are necessary in order to achieve full conversion. To investigate the exact amount of Et₃SiCl needed for the above obtained E-2, a trial run was conducted in sub-gram scale. This helps to avoid the formation of certain undesirable byproducts.

To a solution of compound E-2 (393 mg, 0.5 mmol) in DMF (2.0 mL) was added imidazole (75 mg, 1.1 mmol) and Et₃SiCl (83 mg, 0.55 mmol, 92 μL, 1.1 eq.). The reaction solution was stirred at RT and monitored by TLC. (note: To monitor the reaction by TLC, an aliquot was taken and partitioned in a small amount of methyl tert-butyl ether/water. The organic phase was used for TLC). After 1 h, TLC shows a significant amount of E-2 present and the reaction was stalled. Therefore Et₃SiCl (83 mg, 0.55 mmol, 92 μL, 1.1 eq.) was added. After additional 30 min, TLC showed conversion improved but not complete. Imidazole (38 mg, 0.55 mmol) and Et₃SiCl (46 μL, 0.55 eq) were added. After additional 30 min, TLC showed the reaction was complete. The mixture was quenched with water and extracted with methyl tert-butyl ether (2×). The organic layers were combined, dried over Na₂SO₄, filtered, and concentrated in vacuo. Purification of the residue on a 25 G silica gel column with 25-50% EtOAc/hexane provided compound E-3 as a white solid (305 mg, 68% yield).

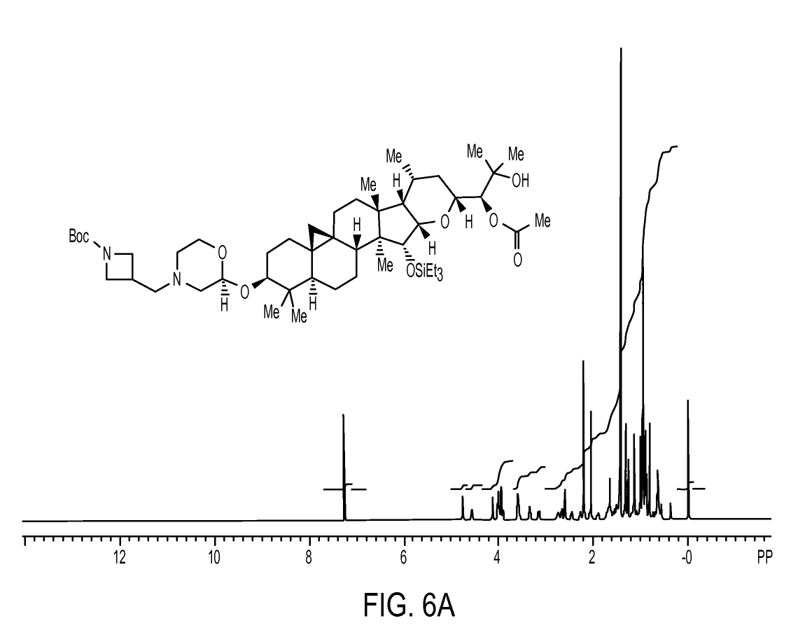

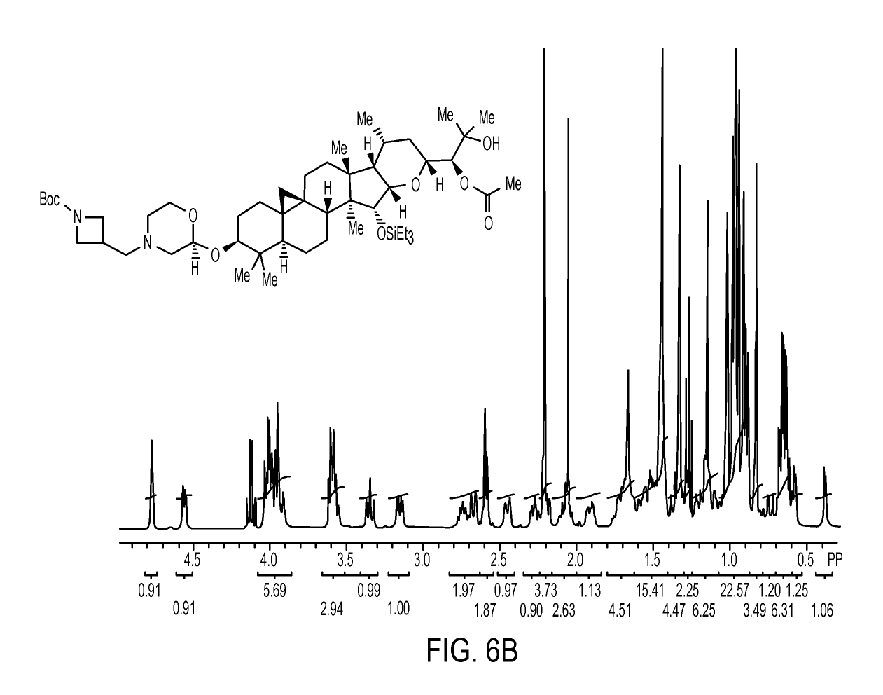

Based on the above trial run, it was determined that 2.75 eq. of Et₃SiCl would be needed to reach full conversion for this batch of E-2. To a solution of compound E-2 (21.5 g, 27.4 mmol) in DMF (100 mL) was added imidazole (6.15 g, 90.4 mmol) and Et₃SiCl (12.7 mL, 75.4 mmol). The reaction solution was stirred at rt for 20 min, at which point TLC indicated the reaction was complete. The reaction mixture was partitioned between methyl tert-butyl ether (400 mL) and H₂O (200 mL). The layers were separated, and the aqueous layer was extracted with methyl tert-butyl ether (200 mL, 100 mL). The combined organic layers were dried over Na₂SO₄, filtered, and concentrated in vacuo. Purification of the residue on a 340 G silica gel column with 25-50% EtOAc/hexane provided compound E-3 as a white solid (16.4 g, 67% yield). (FIGS. 6a & 6b)

### Step S-7

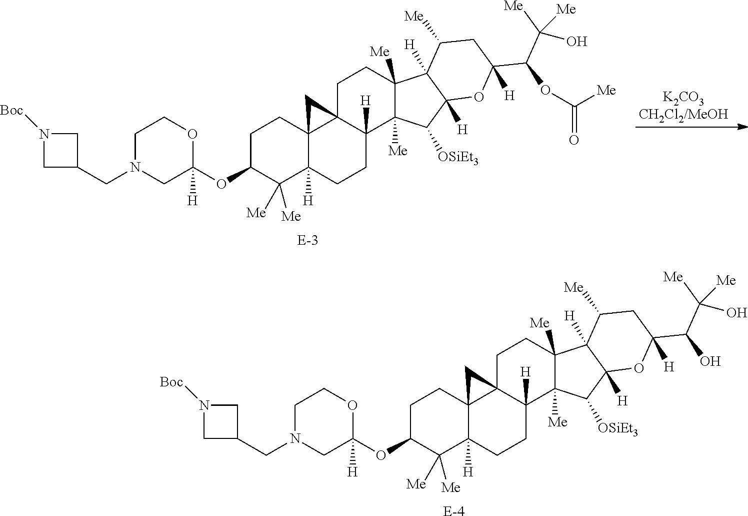

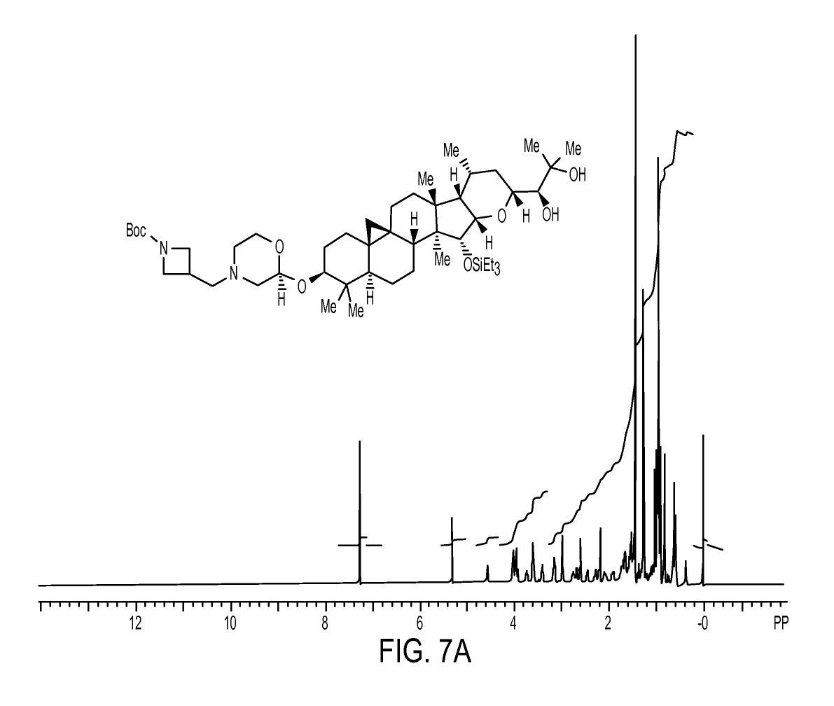

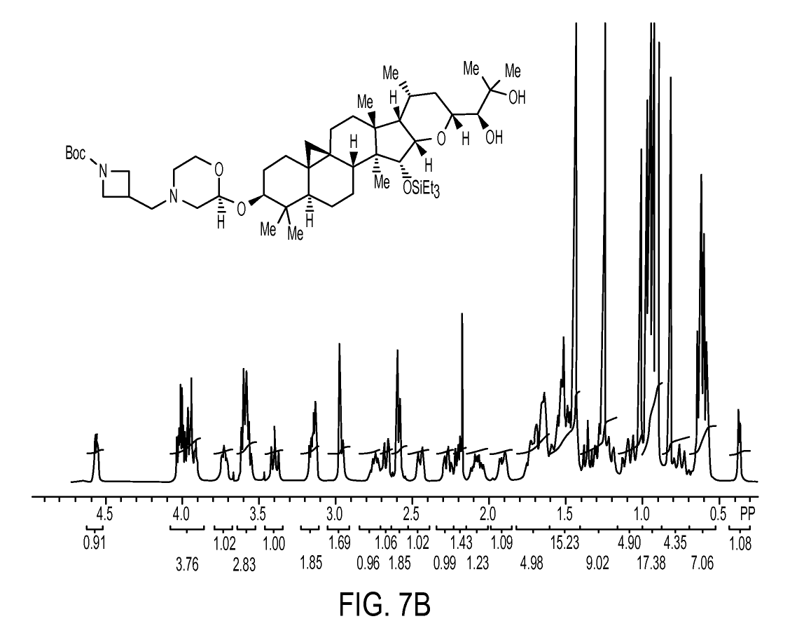

A solution of compound E-3 (16.4 g, 18.3 mmol) in CH₂Cl₂ (61 mL) and MeOH (61 mL) was treated with K₂CO₃ (17.7 g, 128.1 mmol). The reaction was stirred at RT and monitored by LC/MS. (note: To monitor the reaction by LC/MS, an aliquot was taken and diluted with MeOH. Two drops of 10% HCl was added to remove the TES group. The resulting solution was used for LC/MS. See attachment). After 4 h, the starting material was completely consumed, and the major product observed was the desired alcohol E-4 (m/z M+1, 745). The reaction was then diluted with CH₂Cl₂ (300 mL) and H₂O (300 mL), and stirred at RT for 20 min. The layers were separated, and the aqueous layer was extracted with CH₂Cl₂ (100 mL, 2×50 mL). The combined organic layers were dried over Na₂SO₄, filtered, concentrated, and dried under high vacuum overnight. Diol E-4 was obtained as a white solid (15 g, 96% yield) and was used in the next step without purification. (FIGS. 7a & 7b)

### Step S-8

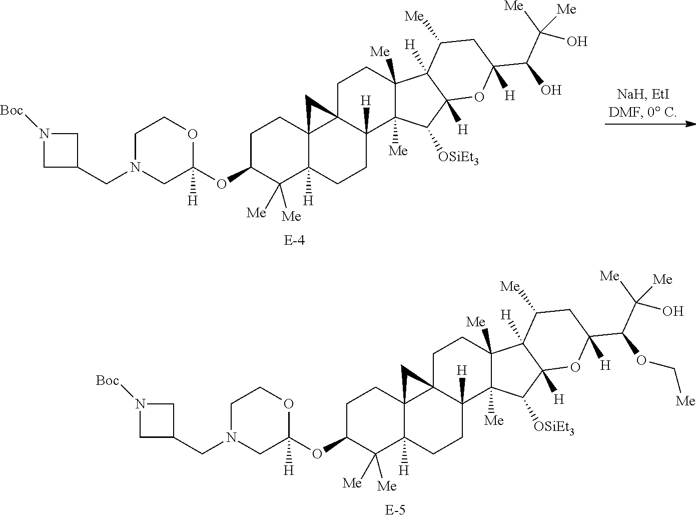

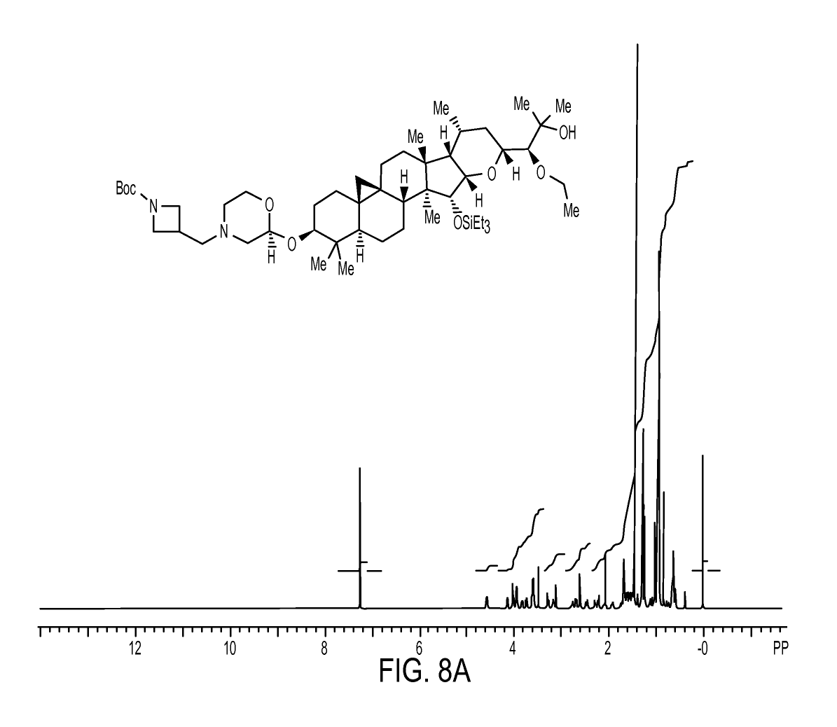

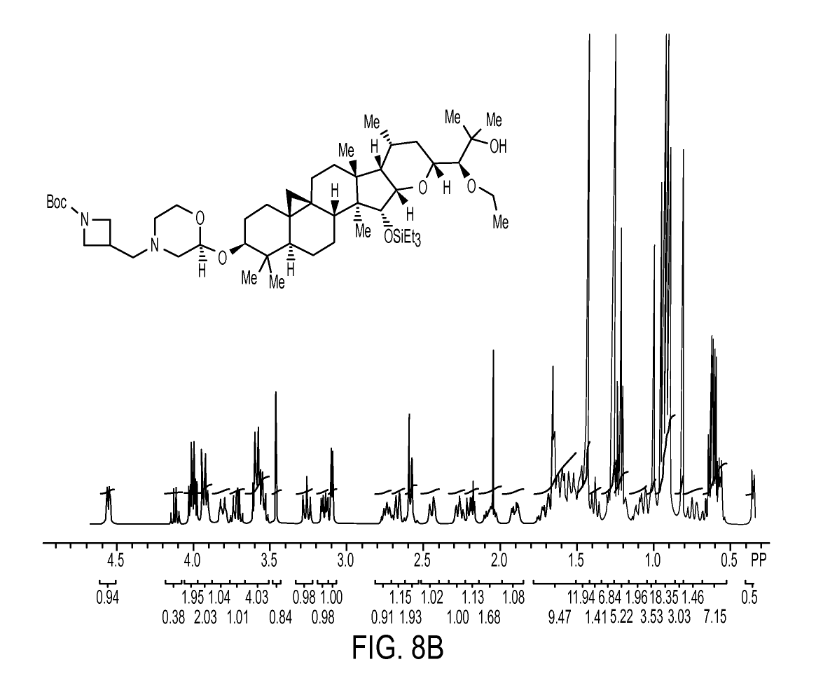

A solution of diol E-4 (15 g, 17.5 mmol) in DMF (87 mL) was cooled to 0° C. and treated with NaH (3.49 g, 60% dispersion in mineral oil, 87.3 mmol) portion-wise (Caution: gas evolution). The solution was stirred at 0° C. for 5 min and then EtI (3.5 mL, 43.8 mmol) was added dropwise. The reaction was closely monitored by LC/MS (note: To monitor the reaction by LC/MS, an aliquot was taken and diluted with MeOH. Two drops of 10% HCl were added to remove the TES group. The resulting solution was used for LC/MS. See attachment). After 35 min, LC/MS analysis shows the completion of the reaction, whereupon the reaction was carefully quenched at 0° C. with sat. aqueous NH₄Cl (100 mL) (Caution: gas evolution) and transferred to a 500 mL separatory funnel charged with methyl tert-butyl ether (200 mL). The organic layer was removed, washed with H₂O (2×50 mL), and collected. The aqueous layers were combined and extracted with methyl tert-butyl ether (2×50 mL). All organic layers were combined, dried over Na₂SO₄, filtered, and concentrated in vacuo. Purification of the residue on a 340 G silica gel column with 25% EtOAc/hexane provided compound E-5 as a white solid (11.7 g, 76% yield). (FIGS. 8a & 8b)

### Step S-9

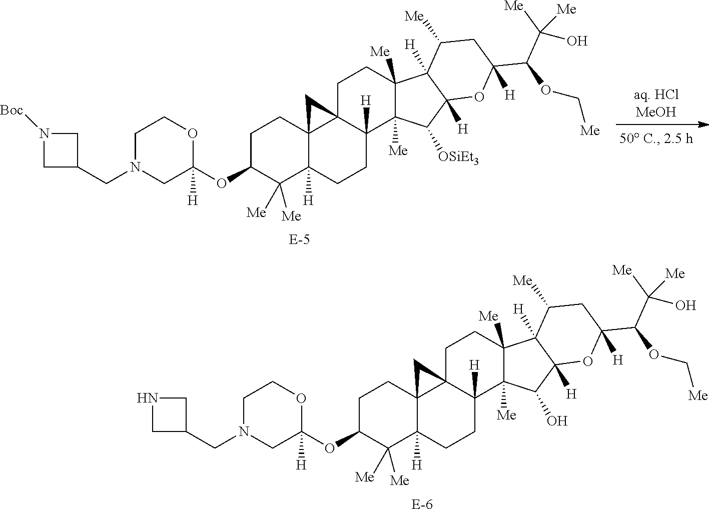

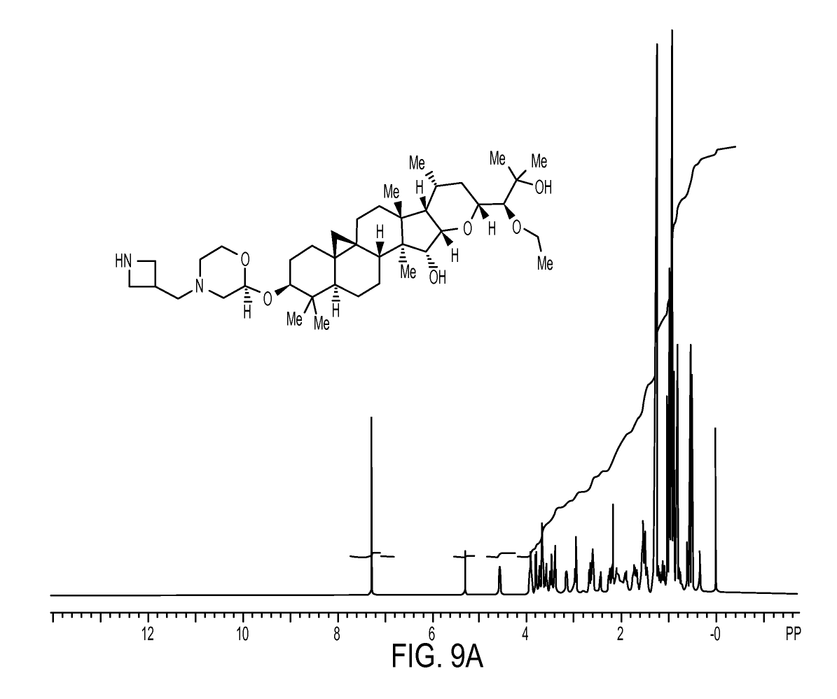

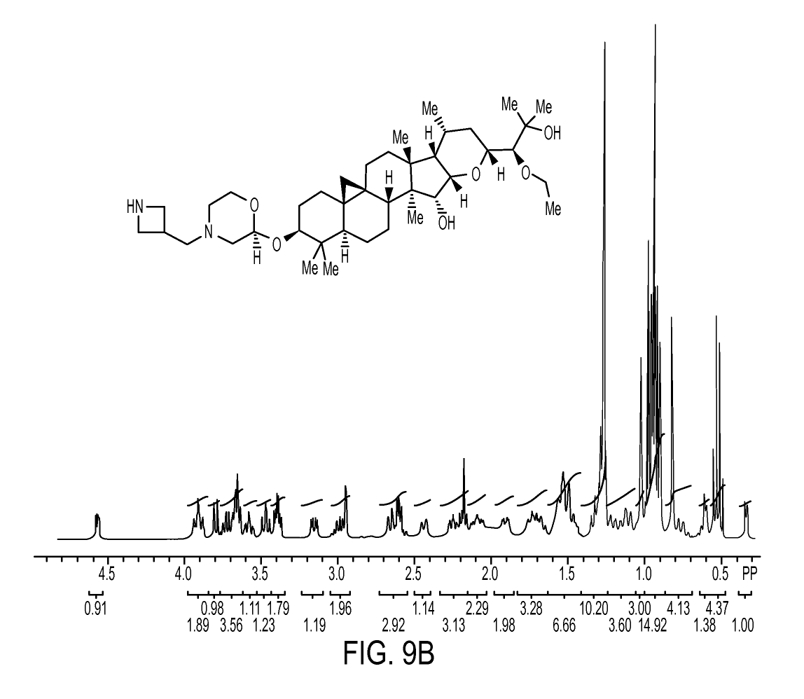

To a solution of carbamate E-5 (519 mg, 0.59 mmol) in MeOH (3.0 mL) was added aq. HCl (2.0 M, 3.0 mL, 6 mmol). The resulting solution was stirred at 50° C. and monitored by LC/MS. After 2.5 h, LC/MS analysis showed the complete cleavage of the starting material to the desired product (M+1, 673). The mixture was diluted with CH₂Cl₂ (50 mL) and washed with aq. NaOH (5 M, 6 mL). The aqueous layer was extracted with CH₂Cl₂ (10 mL). The combined organic layers were dried over Na₂SO₄, filtered, and concentrated in vacuo. Compound E-6 was obtained as a white solid (free base, 440 mg) and used in the next step without purification. (FIGS. 9a & 9b)

### Step S-10

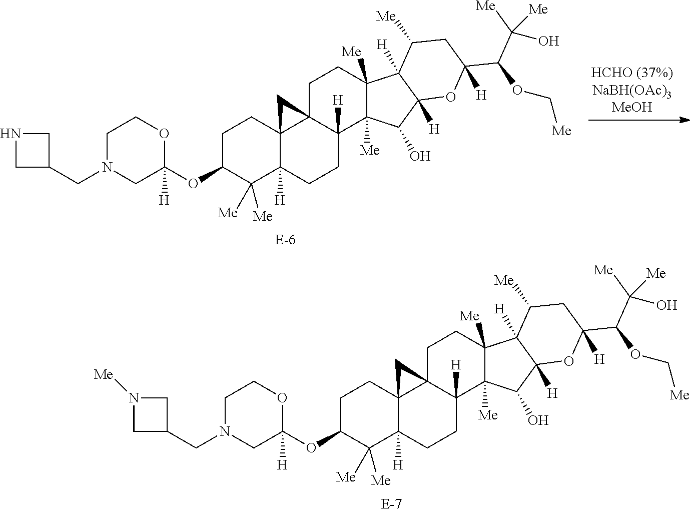

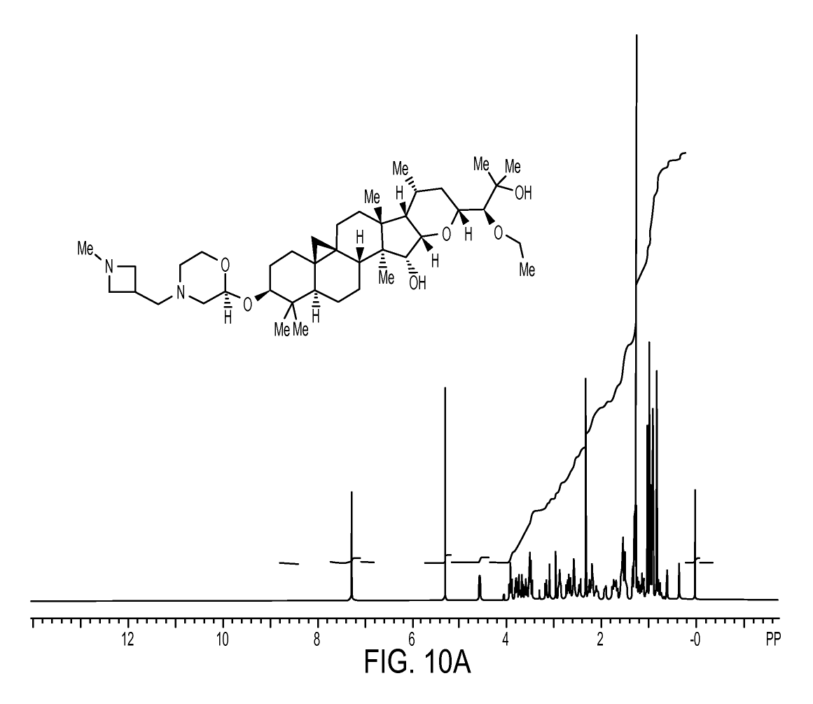

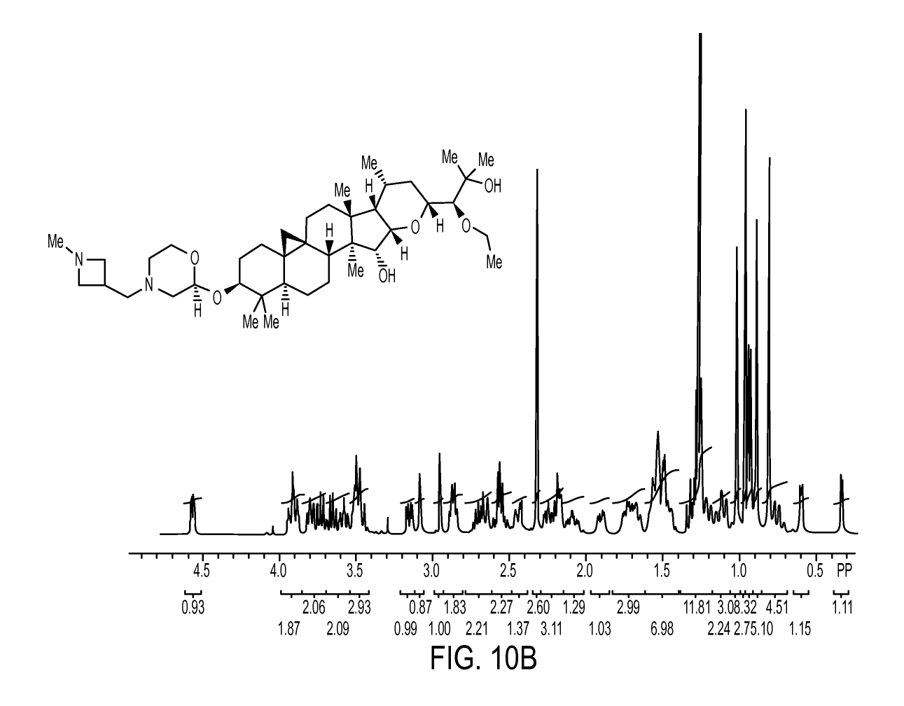

To a solution of amine E-6 (8.4 g, 12.5 mmol) in MeOH (83 mL) was added 37% aq. formaldehyde (1.46 mL, 18.8 mmol) followed by NaBH(OAc)₃ (3.44 g, 16.3 mmol). The mixture was stirred at RT for 20 min, whereupon analysis by LC/MS showed complete conversion of starting material (M+1, 673) to the desired product (M+1, 687). The mixture was then concentrated in vacuo to ˜20 mL, diluted with CH₂Cl₂ (300 mL), transferred to a 1-L separatory funnel, and washed with 1 N aq. NaOH (32.5 mL, 32.5 mmol). The organic layer was collected, and the aqueous layer was extracted with CH₂Cl₂ (150 mL, 50 mL). The combined organic layers were dried over Na₂SO₄, filtered, and concentrated in vacuo. Purification of the residue on a C-18 column (120 G) with 25% MeCN/H₂O (0.1% formic acid) provided compound E-7 as a formic acid salt (7.1 g). The solid was dissolved in CH₂Cl₂ (200 mL) and washed with 1 M KOH (50 mL). The organic layer was collected and the aqueous layer was extracted with CH₂Cl₂ (2×50 mL). The combined organic layers were dried over Na₂SO₄, filtered and concentrated in vacuo to provide free base of E-7 as a white solid (7.0 g, 82% yield). (FIGS. 10a & 10b).

### Example 2

### Step S-3

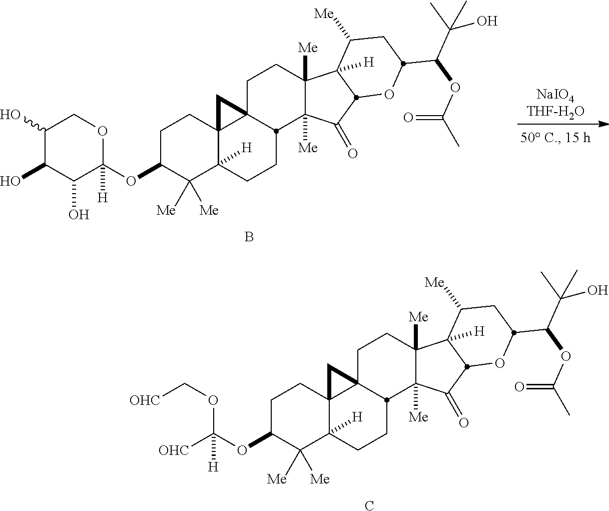

A 1-L one-necked, round-bottomed flask was charged with B (60.97 g, 92 mmol, ˜90% by ELSD), THF (600 mL), water (200 mL) and an egg shaped magnetic stirrer (1¼″×⅝″) and heated in an oil bath held at 50° C. with vigorous stirring (1000 rpm) until all material dissolved. NaIO₄ (78.69 g, 368 mmol, 4 equiv.) was added and stirring was continued until LC/MS indicated the disappearance of the intermediate resulting from mono-oxidative cleavage (m/z=661) after 15 h. The reaction mixture was cooled to room temperature and concentrated under reduced pressure until ˜600 mL of solvent had been removed. The residual slurry was transferred to a 2-L one-necked, round-bottomed flask with dichloromethane (300 mL) and water (300 mL), and stirred at room temperature until all solids were suspended and finely divided after 30 min. The biphasic mixture was transferred to a separatory funnel containing dichloromethane (800 mL) and water (800 mL), 1.0M HCl (300 mL) was added, the phases were homogenized and allowed to separate. The aqueous phase was extracted with dichloromethane (2×w/1000 mL; then 1×w/500 mL), and the combined organic phases were washed with 10% w/v aqueous NaOAc (300 mL). The aqueous phase was back-extracted with dichloromethane (300 mL) and the combined organic phases were dried over Na₂SO₄, filtered and concentrated under reduced pressure to yield crude dialdehyde C as an orange solid foam that was used without further purification, assuming quantitative yield.

### Step S-4

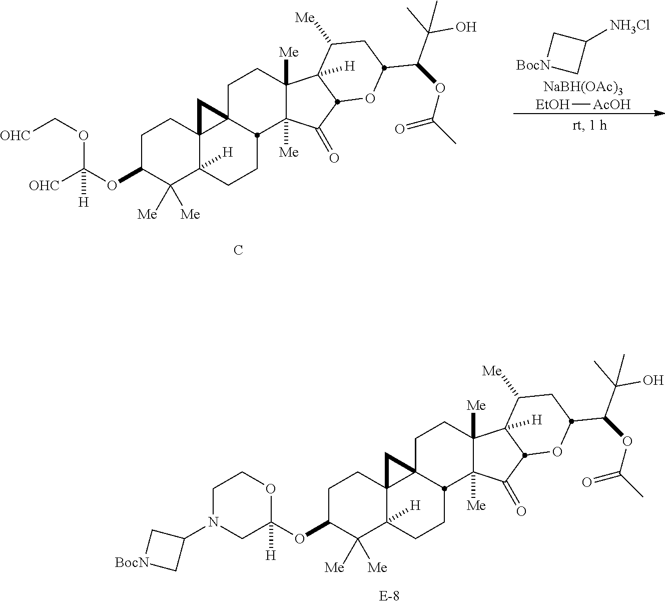

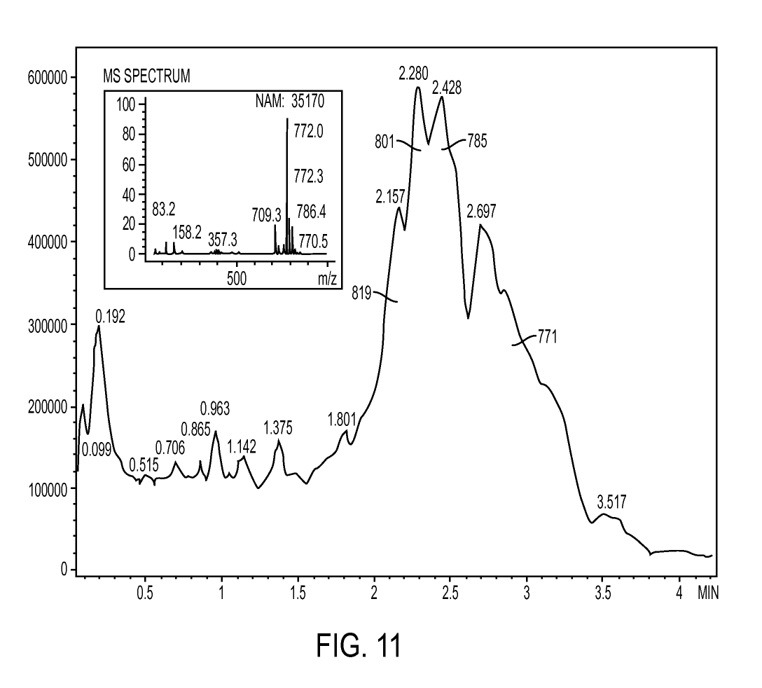

A 1-L one-necked, round-bottomed flask was charged with 1-Boc-3-(amino)azetidine (16.633 g, 97 mmol, 1.05 equiv.) and reagent alcohol (˜90% EtOH, remainder iPrOH, MeOH, 500 mL) and stirred at room temperature while 1M HCl (96 mL, 96 mmol, 1.04 equiv.) was added rapidly dropwise. The resulting solution was concentrated under reduced pressure to yield 20.155 g of the hydrochloride salt as a white powder. A solution of dialdehyde C (assumed ˜92 mmol) in EtOH (540 mL) and AcOH (60 mL) was added and the resulting mixture was stirred at room temperature while sodium triacetoxyborohydride (58.48 g, 276 mmol, 3.0 equiv.) was added in one portion. The reaction was stirred until LC/MS indicated the complete disappearance of starting material by LC/MS (m/z=631) and formation of a new product with the desired mass (m/z=771) after 60 min. The mixture was partitioned between dichloromethane (1 L) and water (1 L), the aqueous phase was extracted twice with dichloromethane (500 mL), and the combined organic phases were dried over Na₂SO₄, filtered and concentrated. The orange viscous oil residue was concentrated twice from toluene (500 mL) to remove residual AcOH and provide 70.873 g of the crude morpholine E-8 as an orange solid foam that was used without further purification, assuming quantitative yield. (FIG. 11)

### Step S-5

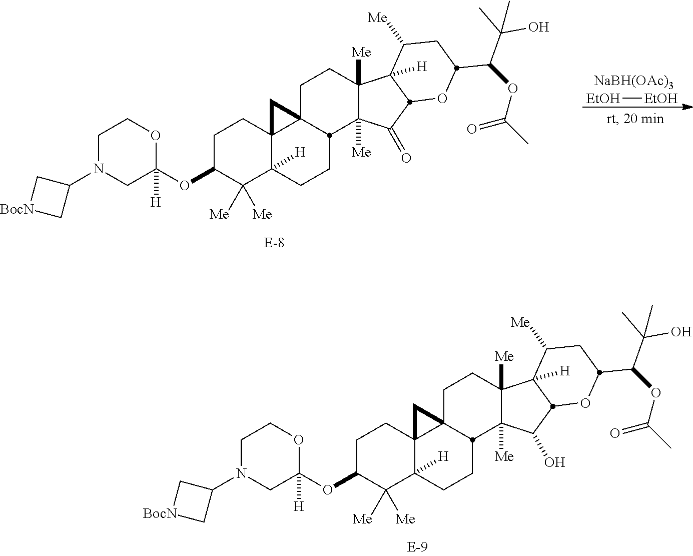

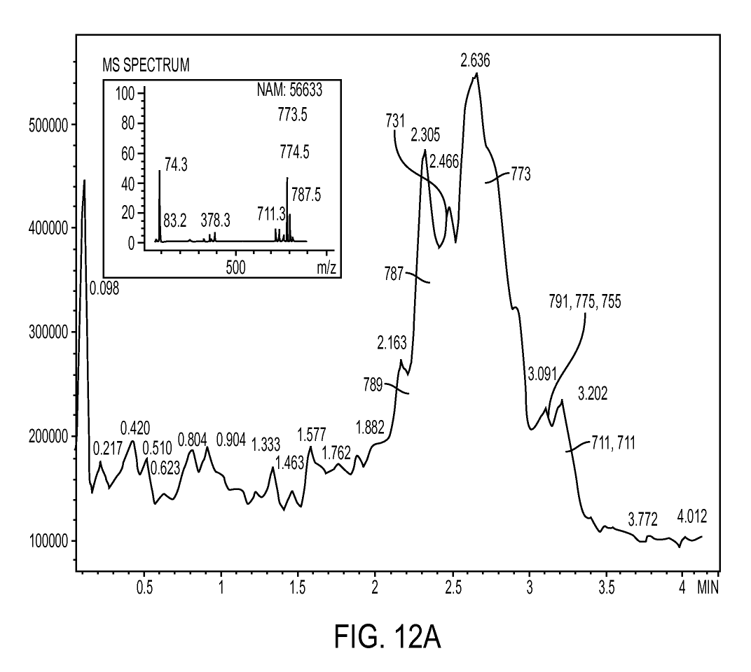

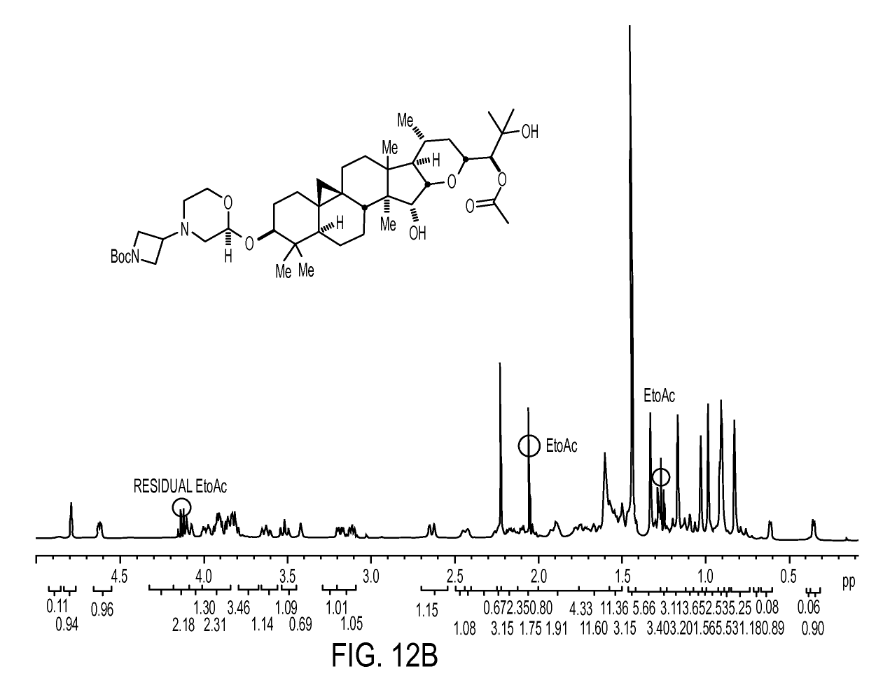

A 1-L one-necked, round-bottomed flask was oven dried and flushed with nitrogen then charged with sodium borohydride (3.828 g, 101 mmol, 1.1 equiv.) and EtOH (61 mL) and the resulting mixture was stirred at room temperature for 10 min (most borohydride was dissolved, but not all). A solution of ketone E-8 (assumed ˜92 mmol) in EtOAc (610 mL) was added over 1 minute (Note: gas evolution) and the mixture was stirred until LC/MS indicated complete consumption of the starting material (m/z=771) and formation of the desired product (m/z=773) after 20 minutes. The resulting mixture was cooled at 0° C. and AcOH (17.4 mL, 18.3 g, 304 mmol, 3.3 equiv.) was added dropwise over minutes (Caution: vigorous gas evolution!), stirred for 5 minutes, then partitioned between dichloromethane (1 L) and water (1 L). The aqueous phase was extracted three times with dichloromethane (0.5 L each), then the combined organic phases were dried over Na₂SO₄, filtered, and concentrated under reduced pressure. The residue was concentrated twice from toluene (500 mL) to give an orange solid foam. The crude product was dissolved in dichloromethane (200 mL) and applied to the top of a 750 g silica gel column (Biotage SNAP XL, CV=990 mL) and eluted (1 CV 25% EtOAc-hexanes, 8 CV 25-100% EtOAc-hexanes, 3 CV 100% EtOAc; Collected 4.25 L of forerun, then 50 mL fractions). Fractions 38-150 were combined and concentrated to give 34.577 g of the desired alcohol E-9 as pale yellow solid (49% over three steps, NMR shows ˜90% pure). (FIGS. 12a & 12b)

### Step S-6

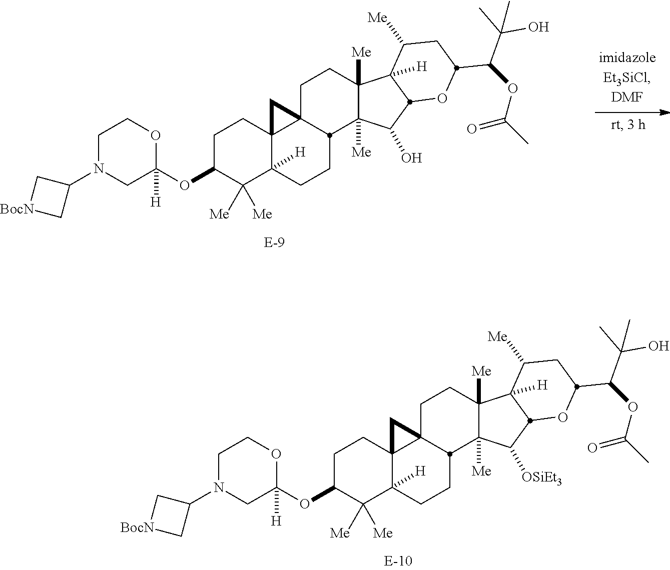

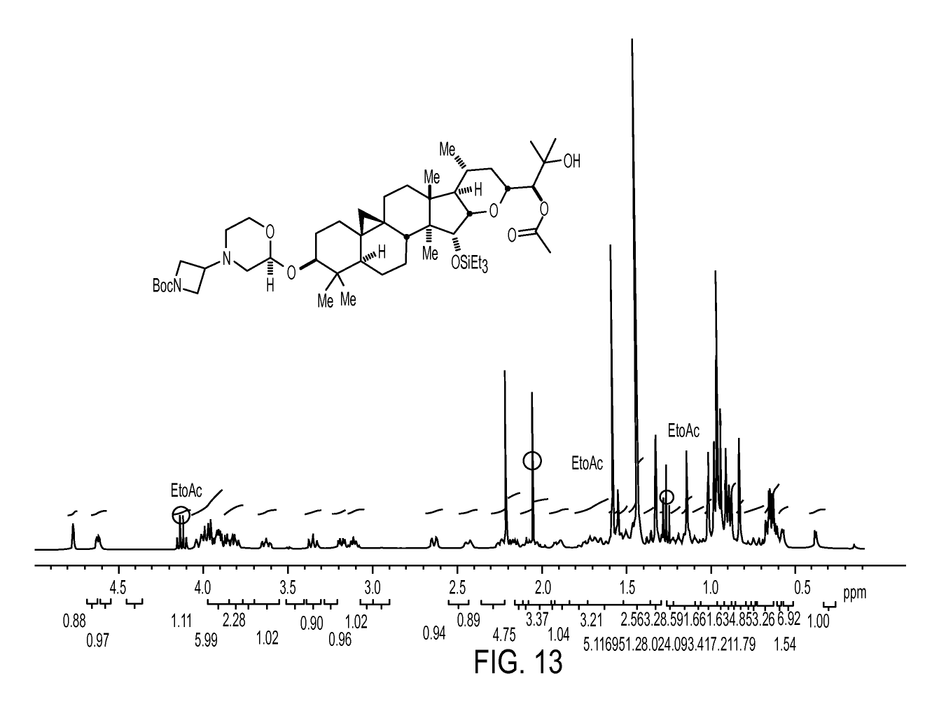

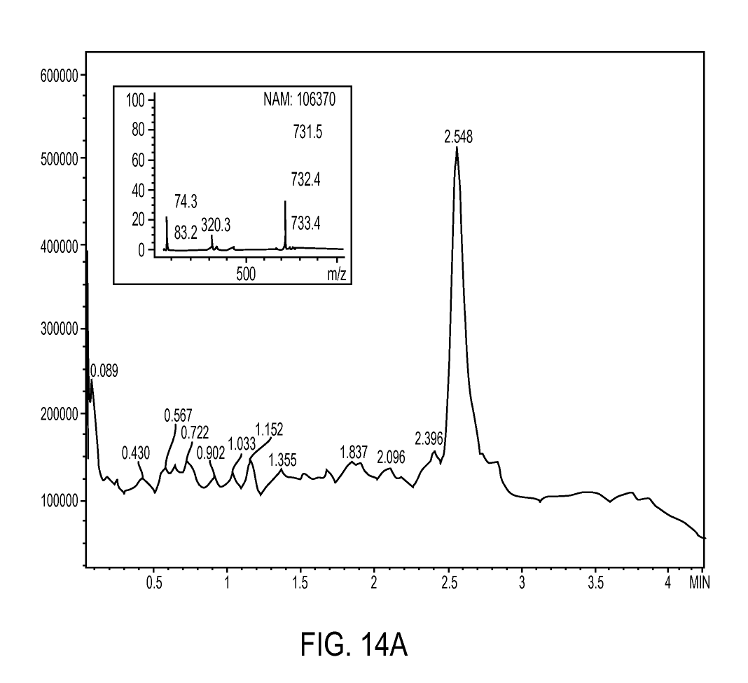

A 500-mL one-necked, round-bottomed flask was oven-dried and flushed with nitrogen then charged with C₁₋₅-alcohol E-9 (34.577 g, 45 mmol) and DMF (179 mL) and stirred at room temperature while imidazole (7.31 g, 108 mmol, 2.4 equiv.) was added. The reaction was stirred 5 minutes, then chlorotriethylsilane (9.0 mL, 8.1 g, 54 mmol, 1.2 equiv.) was added dropwise over 10 min. The reaction was monitored by TLC (1:1 EtOAc:Hexanes, starting material R_{f}=0.11; Desired product R_{f}=0.65; C-15, C-25 O-silylated product R_{f}=0.85). After 1 h partial conversion was observed, but after 2 h no further conversion was observed, so additional imidazole (0.73 g, 11 mmol, 0.24 equiv.) and chlorotriethylsilane (0.90 mL, 0.81 g, 5.4 mmol, 0.12 equiv.) was added. The reaction was stirred 30 min, (TLC indicates trace starting material, presence of bis silyl ether) then partitioned between MTBE (1.5 L) and half-saturated aqueous NaHCO₃ (400 mL) and the layers separated. The organic phase was washed with saturated aqueous NaHCO₃ (300 mL), twice with water (300 mL each), and brine (300 mL) then dried over Na₂SO₄, filtered and concentrated. The crude product was dissolved in dichloromethane (50 mL) and applied to the top of a 340 g silica gel column (Biotage SNAP, CV=510 mL) and eluted (5 CV 15% EtOAc-hexanes, 5 CV 15-45% EtOAc-hexanes, 4 CV 45% EtOAc-hexanes; Collected 1.5 L of forerun, then 20 mL fractions). Fractions 98-228 were combined and concentrated to give 29.8465 g of silyl ether E-10 as a pale yellow solid (75%). (FIG. 13)

### Step S-7

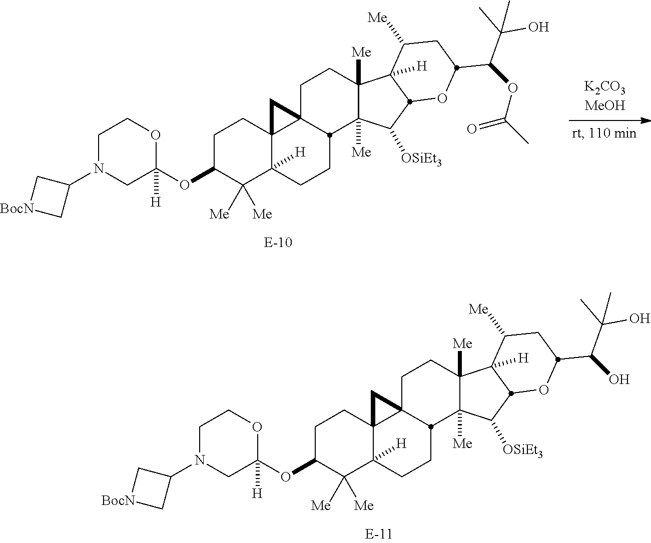

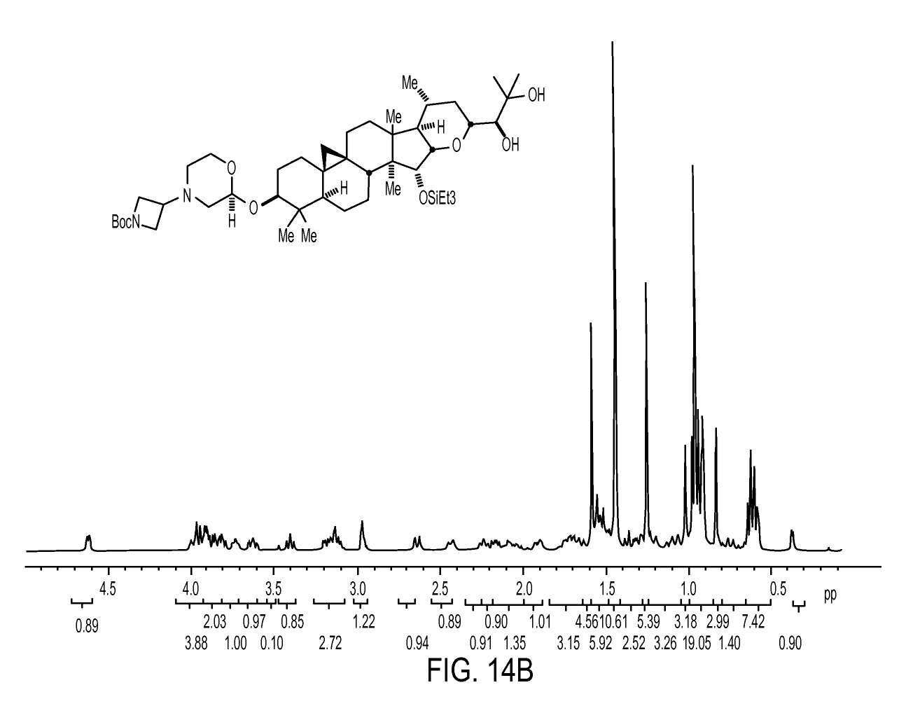

A 500-mL one-necked, round-bottomed flask was charged with C24 acetate E-10 (29.847 g, 34 mmol), dichloromethane (110 mL) and MeOH (110 mL) and the mixture was stirred at room temperature while potassium carbonate (23.26 g, 168 mmol, 5 equiv.) was added. The reaction was stirred at room temperature until LC/MS (10 uL reaction aliquot diluted with dichloromethane and treated with a drop of conc. HCl; starting material m/z=773; product m/z=731) indicated complete consumption of starting material after 110 min. The reaction mixture was partitioned between dichloromethane (500 mL) and saturated aqueous NaHCO₃ (500 mL). The aqueous phase was extracted with dichloromethane (250 mL) and the combined organic phases were washed with brine (500 mL), dried over Na₂SO₄, filtered, and concentrated. The residue was dried under vacuum overnight (<1 mmHg) to provide 28.432 g of the diol E-11 as a white powder that was used without further purification. (FIGS. 14a & 14b)

### Step S-8

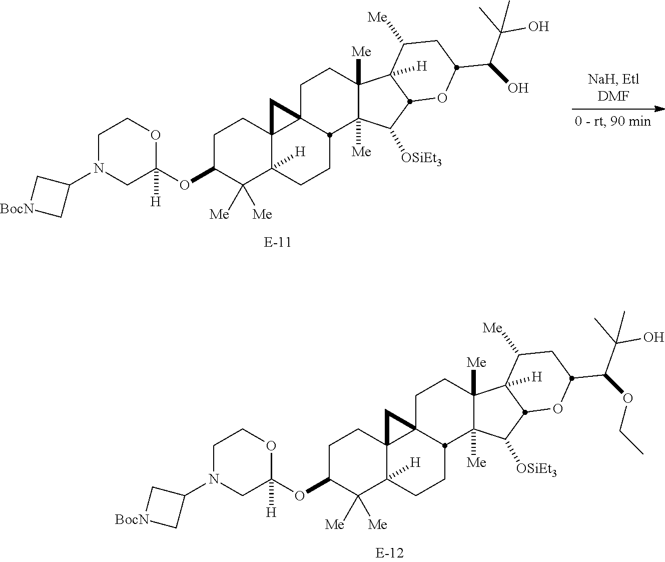

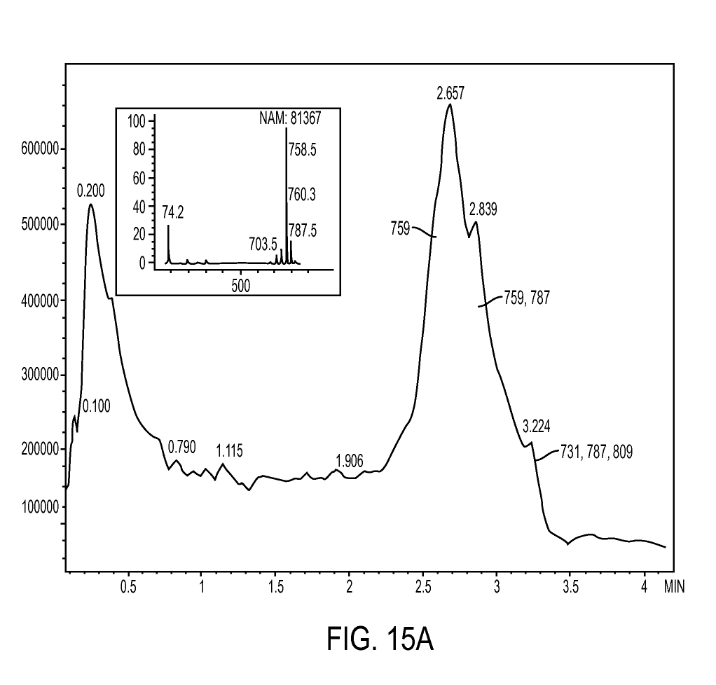

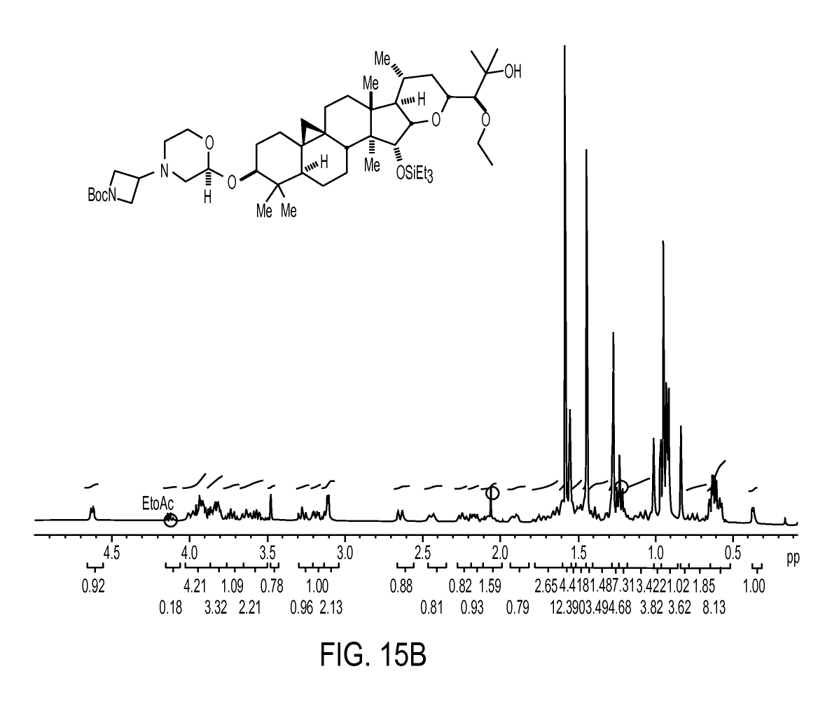

A 500-mL one-necked, round-bottomed flask was charged with C24, C25-diol E-11 (28.0943 g, 33 mmol) and toluene (250 mL) and concentrated under reduced pressure to remove traces of water, and the flask was backfilled with nitrogen. The residue was dissolved in DMF (277 mL) and the mixture was cooled to 0° C. and sodium hydride (6.65 g, 60% dispersion in mineral oil, 166 mmol, 5 equiv.) was added. The mixture was stirred 5 minutes, then iodoethane (6.6 mL, 12.87 g, 82.5 mmol, 2.5 equiv.) was added and the reaction stirred while warming slowly until LC/MS (10 uL reaction aliquot diluted with dichloromethane and treated with a drop of conc. HCl; SM m/z=731; product m/z=759; C15, C24 OEt m/z=787) indicated most starting material has been consumed and presence of diether was observed after 90 minutes. The mixture was partitioned between MTBE (2 L) and saturated aqueous ammonium chloride (600 mL). The organic phase was washed twice with water (300 mL) and then with brine (300 mL), dried over Na₂SO₄, filtered and concentrated to provide a yellow solid. The residue was dissolved in dichloromethane (50 mL) and applied to the top of a 340 g silica gel column (Biotage SNAP, CV=510 mL) and eluted (10 CV 15% EtOAc-hexanes, 5 CV 15-40% EtOAc-hexanes, 5 CV 40% EtOAc-hexanes; Collected 1.5 L of forerun, then 50 mL fractions). Fractions 36-125 were combined and concentrated to give 18.919 g of ethyl ether E-12 as a white powder (65% over two steps). (FIGS. 15a & 15b)

### Step S-9

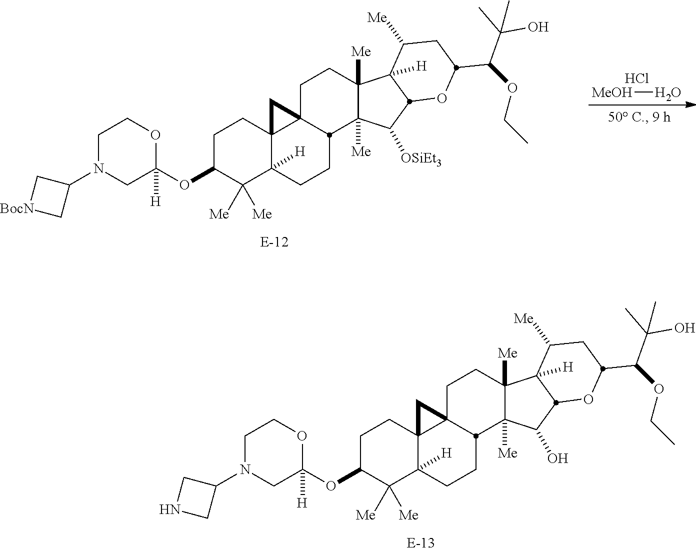

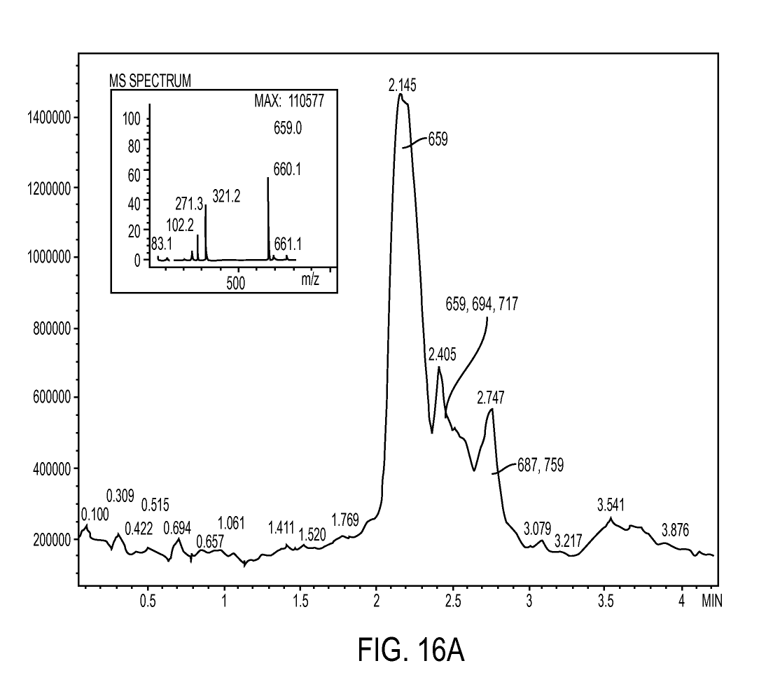

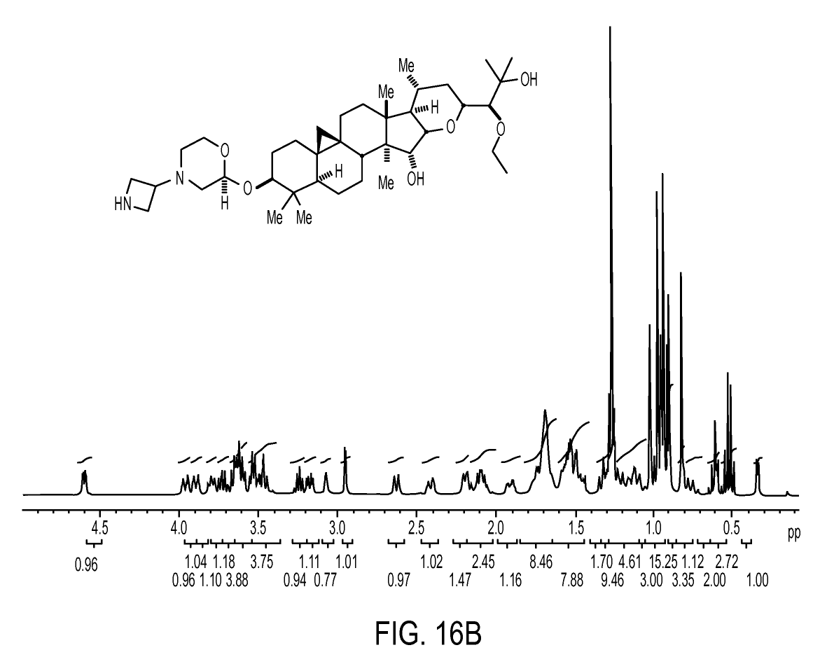

A 500-mL one-necked, round-bottomed flask was charged with N-Boc carbamate E-12 (18.919 g, 22 mmol), and MeOH (163 mL) and a 1.0M solution of HCl in 1:1 MeOH:H2O (217 mL, 217 mmol, 10 equiv.) was added. The resulting mixture was heated at 50° C. until LC/MS indicated no N-Boc carbamate remaining (NBoc m/z=759; NH m/z=659) after 9 h. The reaction was allowed to cool to room temperature and was concentrated under reduced pressure until ˜200 mL of solvent was removed. The residue was diluted with dichloromethane (1.5 L) and a solution of sodium hydroxide (6.1 M, 178 mL, 1085 mmol, 50 equiv.) was added. The aqueous phase was extracted four times with dichloromethane (500 mL each) and the absence of desired product was confirmed by LC/MS, then the combined organic phases were dried over Na₂SO₄, filtered and concentrated to provide 15.207 g of E-13 as a white powder. (FIGS. 16a & 16b)

### Step S-10

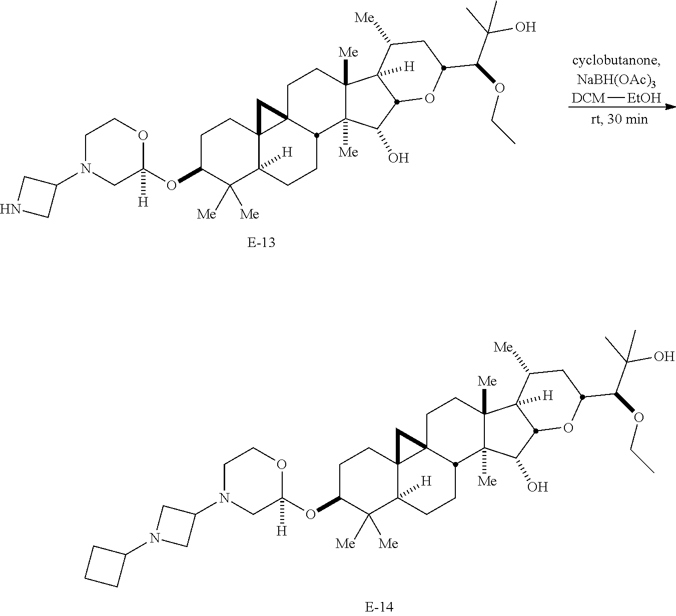

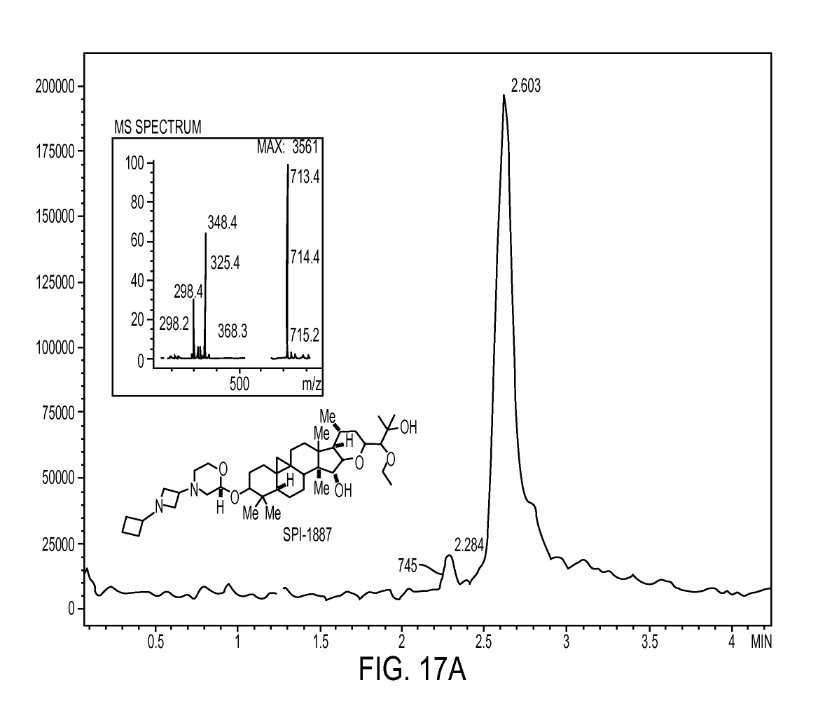

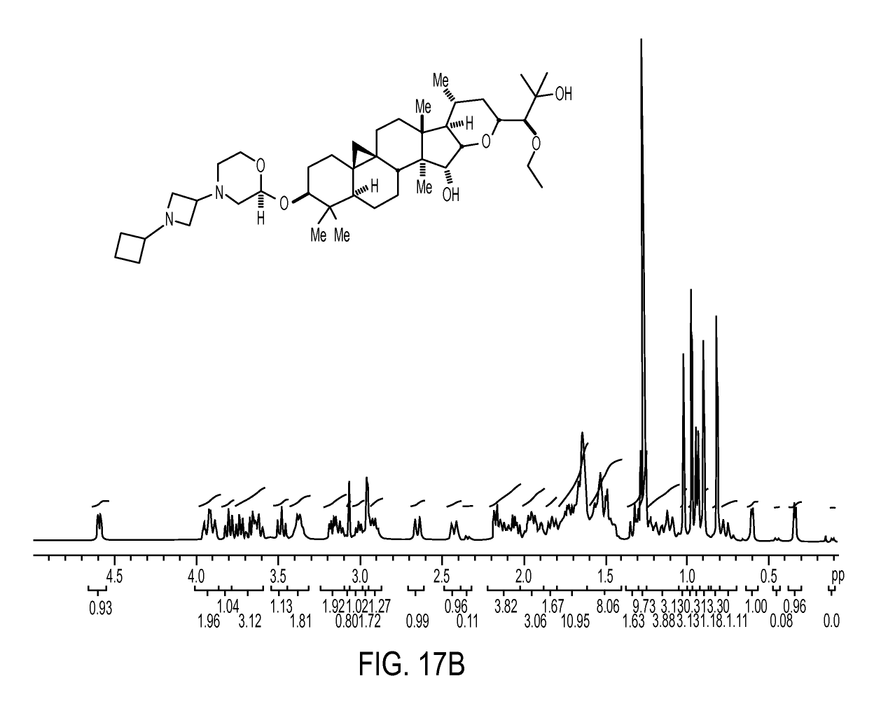

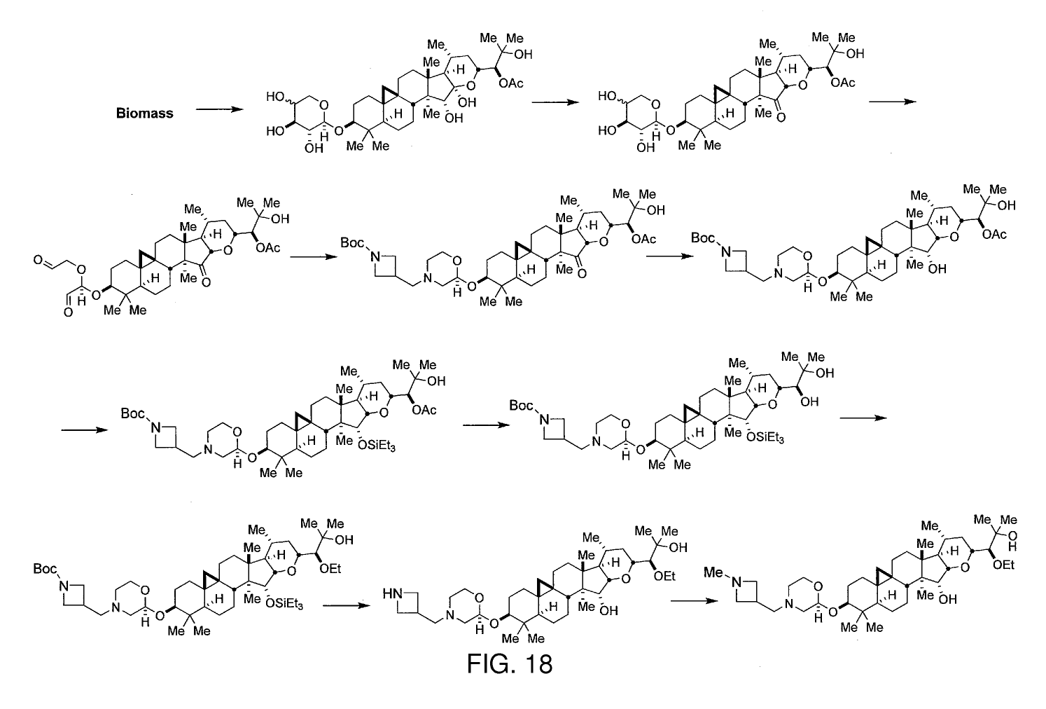

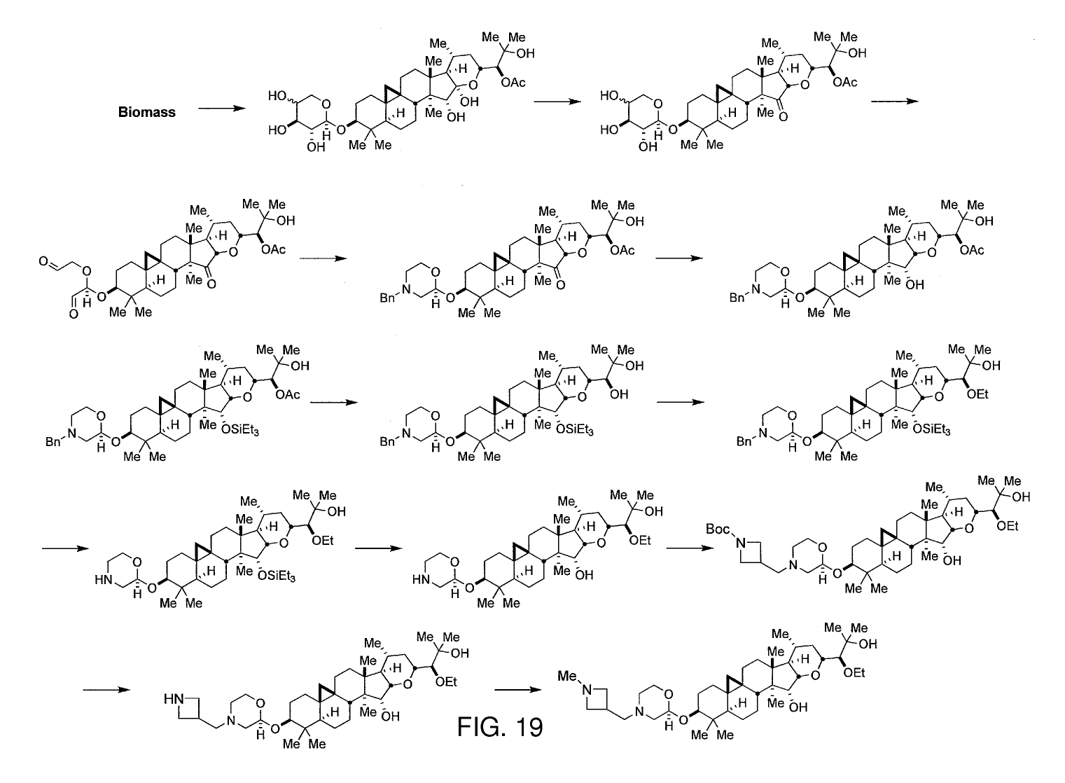

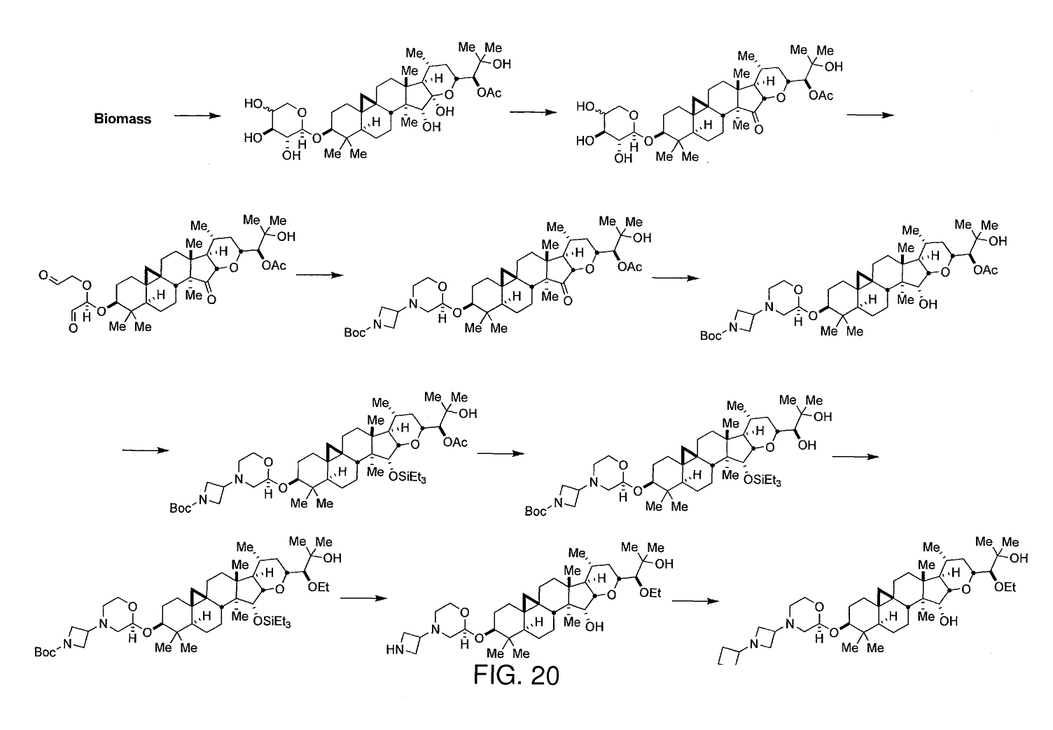

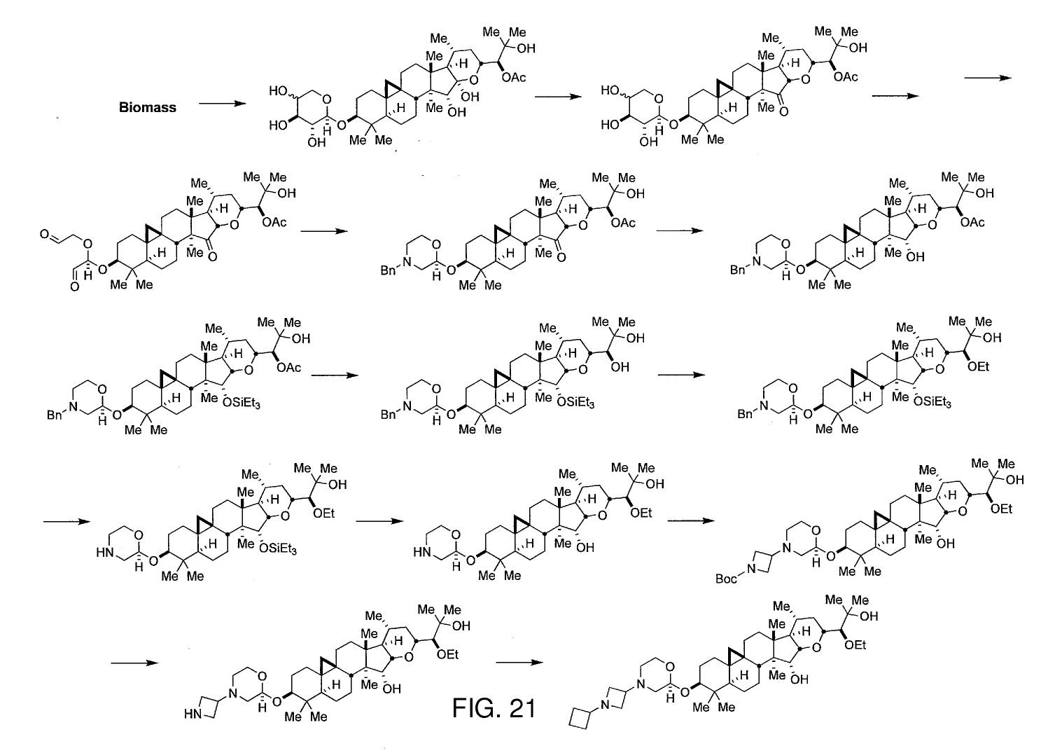

A 500-mL one-necked, round-bottomed flask was charged with amine E-13 (15.206 g, 22 mmol) EtOH (28 mL) and dichloromethane (185 mL) and stirred at room temperature while cyclobutanone (4.0 mL, 3.75 g, 54 mmol, 2.5 equiv.) was added, followed by sodium triacetoxyborohydride (13.78 g, 65 mmol, 3 equiv.). The mixture was stirred until LC/MS shows no starting material remaining (starting material m/z=659, product starting material=713) after 30 minutes, then was partitioned between dichloromethane (1.5 L) and saturated aqueous NaHCO₃ (400 mL). The organic phase was washed with saturated aqueous NaHCO₃ (300 mL) and the combined aqueous phases were extracted with dichloromethane (400 mL). The combined organic phases were dried over Na₂SO₄, filtered, and concentrated, then the residue was dissolved in 50 mL MeOH and applied to the top of a 400 g C18 column (Biotage, CV=510 mL) and eluted (1CV 15% MeCN—H₂O+0.1% formic acid; 10 CV 15-55% MeCN—H₂O+0.1% formic acid; 3CV 55% MeCN—H₂O+0.1% formic acid; collected forerun of 1.5 L, then 50 mL fractions). Fractions 31-49 were collected and concentrated with the aid of reagent alcohol to a volume of ca 500 mL, and a solution of NaOH (14.5 mL, 3M, 43.5 mmol, 2 equiv.) was added. The mixture was extracted with dichloromethane (1.5 L) and the aqueous phase was extracted four times with dichloromethane (0.5 L). The combined organic phases were washed with brine (350 mL), dried over Na₂SO₄, filtered and concentrated to yield 9.749 g of E-14 as a white solid (63% over two steps). Overall yield is 14.8% over 8 steps. (FIGS. 17a-c).

### Example 3

### Synthesis of Compounds of Formula II Via a Compound of Formula I

### Step S-10a

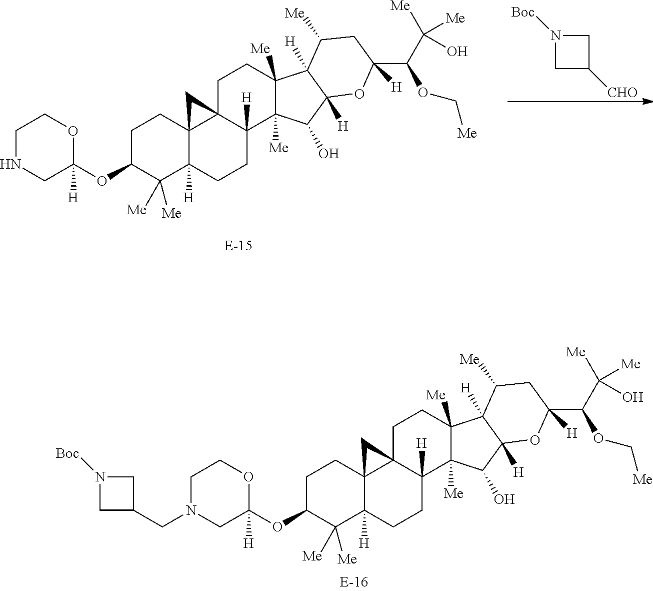

A solution of E-15 (100 mg, 0.166 mmol) in CH₂Cl₂ (0.6 mL) and MeOH (0.3 mL) was treated with 1-Boc-azetidine-3-carboxaldehyde (34 μL, 0.20 mmol) and AcOH (190 μL, 0.33 mmol). The reaction mixture was stirred at rt for 5 min, then NaBH(OAc)₃ (52.8 mg, 0.25 mmol) was added. The reaction was stirred at rt and monitored by LC/MS. After min the reaction mixture was quenched with sat. NaHCO₃ and extracted with CH₂Cl₂ (3 x). The combined organic layers were dried over Na₂SO₄, filtered, and concentrated in vacuo. Purification of the residue on SiO₂ with 5% MeOH in CH₂Cl₂ provided compound E-16 (114 mg) as a white sold in 89% yield. LC/MS [M+1]773.5.

### Step S-10b

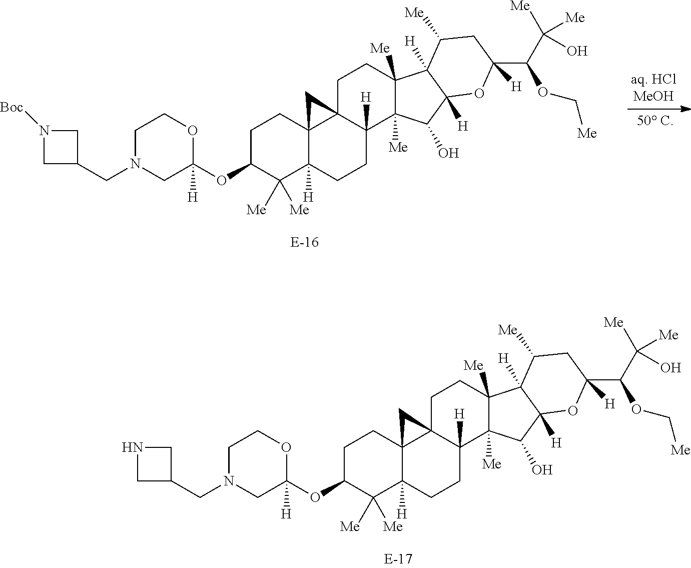

To a solution of E-16 (97 mg, 0.13 mmol) in MeOH (0.5 mL) was added a solution of aq. HCl (2.0 M, 0.5 mL, 1.0 mmol). The resulting solution was stirred at 50° C. and monitored by LC/MS. After 1.5 h, LC/MS analysis showed the complete conversion of the starting material to the desired product (M+1, 673.5). The mixture was diluted with CH₂Cl₂ (2 mL) and washed with aq. KOH (5 M, 1.3 mL). The aqueous layer was extracted with CH₂Cl₂ (3×). The combined organic layers were dried over Na₂SO₄, filtered, and concentrated in vacuo. Compound E-17 was obtained as a white solid (free base, 90 mg) and used in the next step without purification. See, e.g., step S-10 of Example 2, above.

### Example 4

### Step S-10a

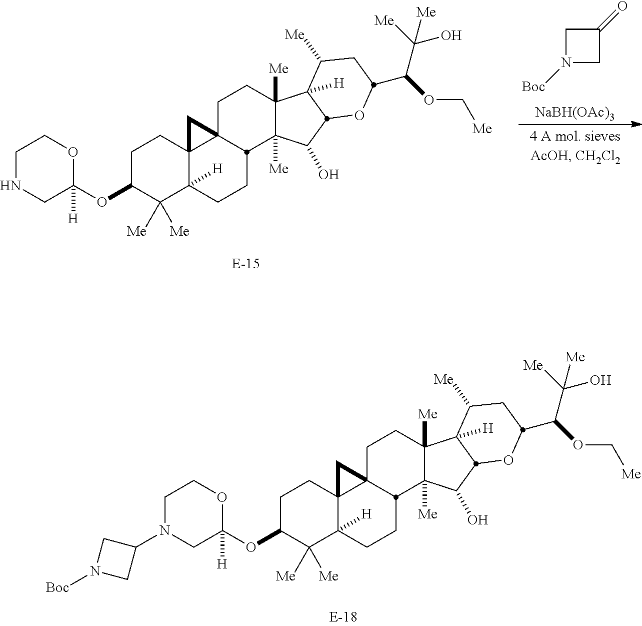

A 50 mL round bottom flask was charged with 4 Å molecular sieves (1.18 g) which were activated by flame-drying under vacuum. The flask was then charged with E-15 (1.007 g, 1.67 mmol), which was dissolved in CH₂Cl₂ (10 mL) and treated with N-Boc-azetidin-3-one (0.572 g, 3.17 mmol) and AcOH (0.20 mL, 3.34 mmol). The reaction was stirred at RT for 2 h, whereupon NaBH(OAc)₃ (0.700 g, 3.17 mmol) was added, and stirring was continued while the progress of the reaction was monitored by LC/MS. After 2½ hours, the reaction was complete as indicated by LC/MS (product m/z [M+H]=759; E-15 m/z [M+H]=604). The solution was poured into CH₂Cl₂/satd. NaHCO₃ (aq.) and the layers were separated. The organic layer was washed with brine, dried (Na₂SO₄), filtered, and concentrated to provide the desired diamine E-18 as a white solid. The crude product was carried on without further purification, assuming quantitative yield.

### Step S-10b

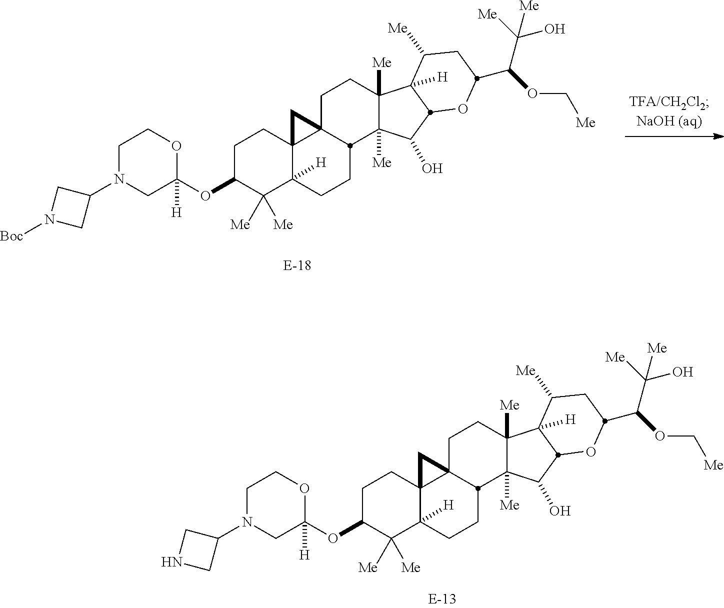

A solution of E-18 in CH₂Cl₂ (5 mL) was treated with trifluoroacetic acid (TFA) (1 mL) and the reaction was stirred at RT, monitoring progress by LC/MS (product m/z [M+H]=659; starting material m/z [M+H]=759). After 30 min, the reaction was not yet complete, so an additional amount of TFA (0.5 mL) was added. After an additional 1 h, the reaction seemed to have stalled, so a mixture of CH₂Cl₂ (3 mL) and TFA (1 mL) was added, and after 1 h longer, an additional portion of TFA (0.5 mL) was added to push the reaction to completion. After 1 h, full consumption of the starting material was observed, so the reaction was poured into CH₂Cl₂/1 M NaOH and the layers were separated. The organic layer was washed with brine, dried (Na₂SO₄), filtered, and concentrated to provide E-13 as a tan solid. The crude product was carried on without further purification.

### Step S-100c

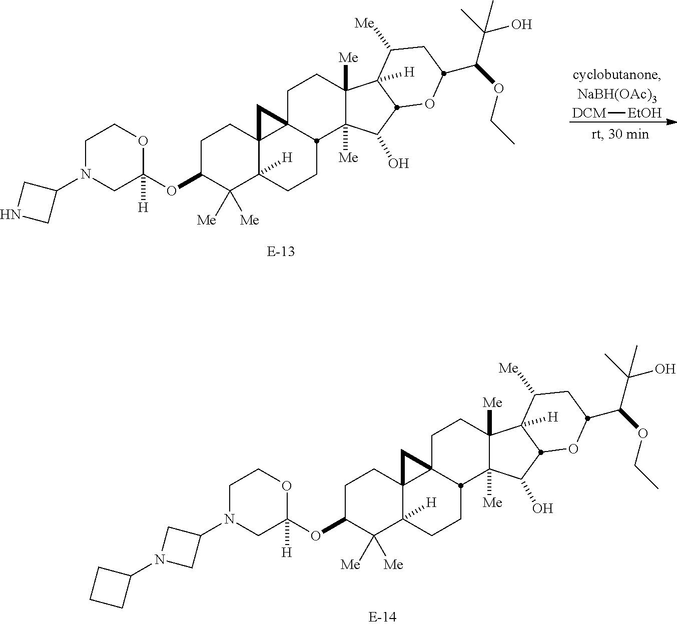

A 500-mL one-necked, round-bottomed flask was charged with E-13 (15.206 g, 22 mmol) EtOH (28 mL) and dichloromethane (185 mL) and stirred at room temperature while cyclobutanone (4.0 mL, 3.75 g, 54 mmol, 2.5 equiv.) was added, followed by sodium triacetoxyborohydride (13.78 g, 65 mmol, 3 equiv.). The mixture was stirred until LC/MS shows no starting material remaining (starting material m/z [M+H]=659; product m/z [M+H]=713) after 30 minutes, then was partitioned between dichloromethane (1.5 L) and saturated aqueous NaHCO₃ (400 mL). The organic phase was washed with saturated aqueous NaHCO₃ (300 mL) and the combined aqueous phases were extracted with dichloromethane (400 mL). The combined organic phases were dried over Na₂SO₄, filtered, and concentrated, then the residue was dissolved in 50 mL MeOH and applied to the top of a 400 g C18 column (Biotage, CV=510 mL) and eluted (1CV 15% MeCN—H₂O+0.1% formic acid; 10 CV 15-55% MeCN—H₂O+0.1% formic acid; 3CV 55% MeCN—H₂O+0.1% formic acid; collected forerun of 1.5 L, then 50 mL fractions). Fractions 31-49 were collected and concentrated with the aid of reagent alcohol to a volume of ca. 500 mL, and a solution of NaOH (14.5 mL, 3M, 43.5 mmol, 2 equiv.) was added. The mixture was extracted with dichloromethane (1.5 L) and the aqueous phase was extracted four times with dichloromethane (0.5 L). The combined organic phases were washed with brine (350 mL), dried over Na₂SO₄, filtered and concentrated to yield 9.749 g of E-14 as a white solid (63% over two steps).

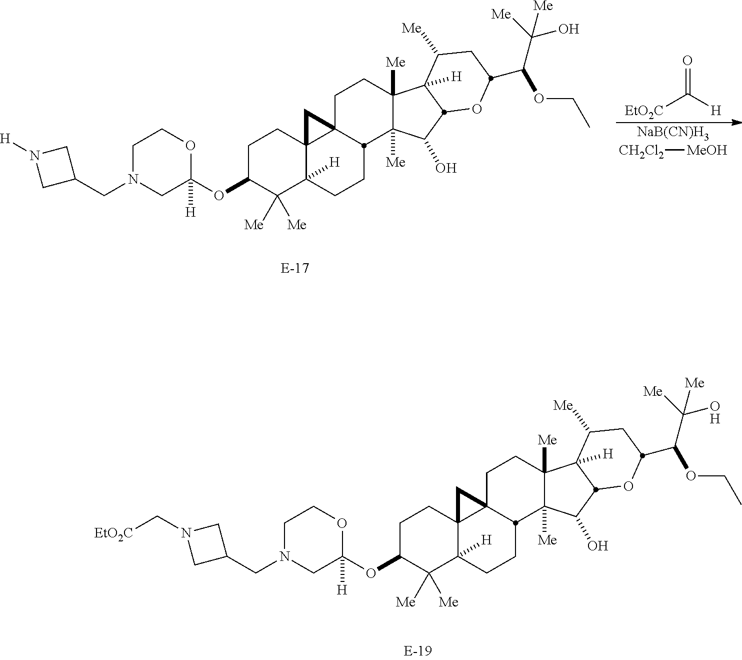

### Example 5

To a solution of E-17 (4.10 g, 6.40 mmol) in a mixture of CH₂CH₂-MeOH (1:1, 100 mL) at room temperature was added ethyl glyoxylate (3.90 g, 50% in toluene, 19.2 mmol) and acetic acid (0.77 g, 0.73 mL, 12.8 mmol.). The reaction mixture was stirred for about 10 min before NaB(CN)H₃ (0.48 g, 7.68 mmol) was added. After 1.5 h, LC/MS showed nearly all the starting material disappeared. The reaction was quenched by NaHCO₃ (saturated solution, 20 mL). The desired product was extracted by CH₂Cl₂ (250 ml, 2×100 ml,). The combined extracts were dried over Na₂SO₄ and concentrated under reduced pressure. The resulting residue was purified by silica gel column chromatography (100 g silica gel column) eluting with a solvent gradient from MeOH/CH₂CH₂ (0:100) to MeOH/CH₂Cl₂ (15:85) to give the desired product E-19 (4.00 g, 82%). LCMS (m/z): [M+H]⁺ 759.5

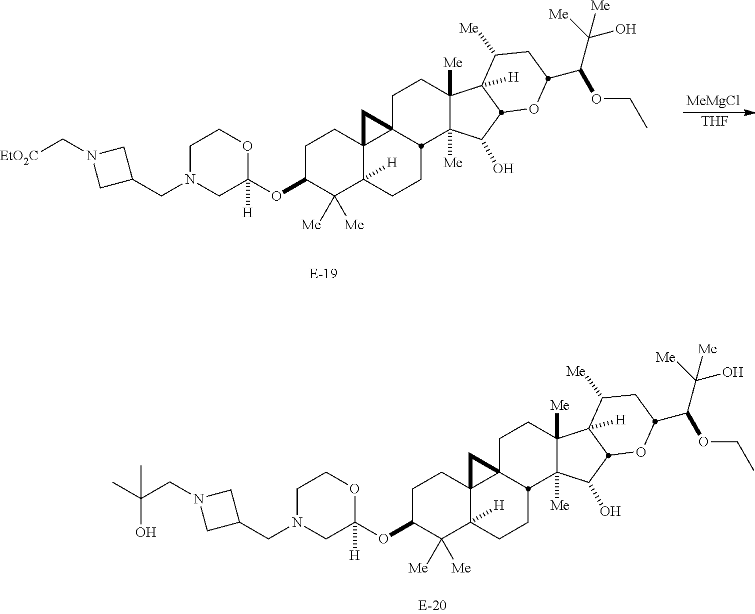

To a solution of E-19 (4.00 g, 5.28 mmol) in THF (80 ml) at 0° C. was added MeMgCl (10.5 ml, 3.0 m in THF, 31.5 mmol). The reaction mixture was stirred at 0° C. for 1 h and then LC/MS showed that all the starting material was consumed. The reaction mixture was quenched by NaHCO₃ (saturated, 30 ml). The desired product was extracted by CH₂Cl₂ (300 mL, 2×200 mL). The combined extracts were dried over Na₂SO₄ and then concentrated under reduced pressure. The resulting residue was subjected to reverse phase flash chromatography (Biotage, 120 g C18 coated column), eluting with a solvent gradient from H₂O/CH₃CN (95:5) to H₂O/CH₃CN (50:50) to provide the desired product as formic acid salts. The solid was dissolved in CH₂Cl₂ (100 ml) and NaOH (1N, 25 mL) was added. After vigorous stirring, the organic layer was separated. The aqueous layer was extracted with CH₂Cl₂ (3×100 mL). The combined organic layers were dried over Na₂SO₄ and concentrated under reduced pressure to give 2.52 g (64%) of the free base E-20. (m/z): [M+H]⁺ 745.6

### Example 6

### Step S-3

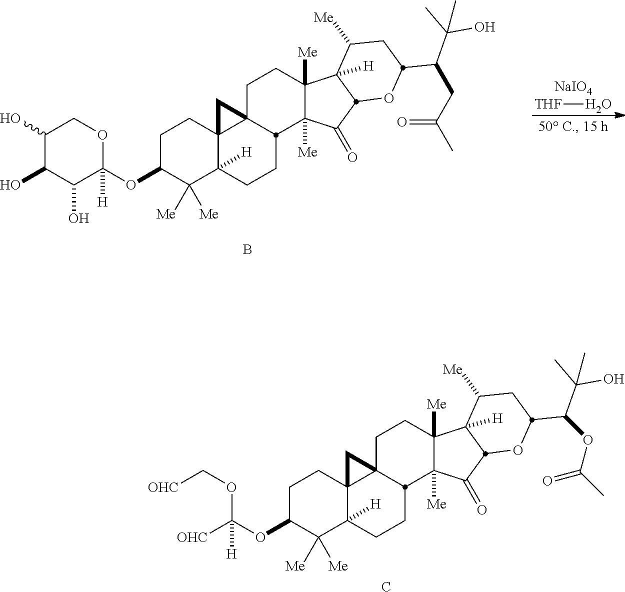

A one-necked, round-bottomed flask was charged with B (1.0 equiv.), THF, water and heated at 50° C. with vigorous stirring until all material dissolved. NaIO₄ (4 equiv.) was added over 60 min. and stirring was continued for 15 h until HPLC indicated the disappearance of starting material. TBME and water were added at 50° C., and the reaction mixture was extracted. The aqueous layer was re-extracted with TBME. The organic layers were washed with sat. NaCO₃, then sat. brine solution. The organic layer was concentrated, and toluene was added, then stripped off. The resulting suspension was diluted with EtOH and THF. The organic phase solution provided crude dialdehyde C as a beige solution with solids, that was used without further purification, assuming quantitative yield.

### Step S-4

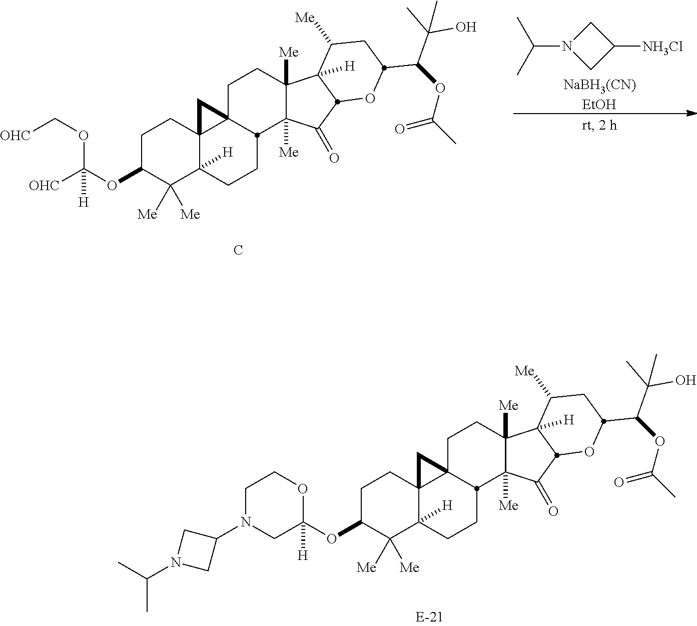

To the reaction mixture above was added 1-isopropylazetidin-3-amine dihydrochloride (1.2 equiv.) and ethanol (0.33 M). The mixture was stirred for 1 h at 20° C., and NaBH₃(CN) (1.2 equiv.) was added over 60 min, and stirring was continued for another 50 min. After 15 hrs, HPLC showed complete conversion of starting material, to provide E-21.

### Step S-5

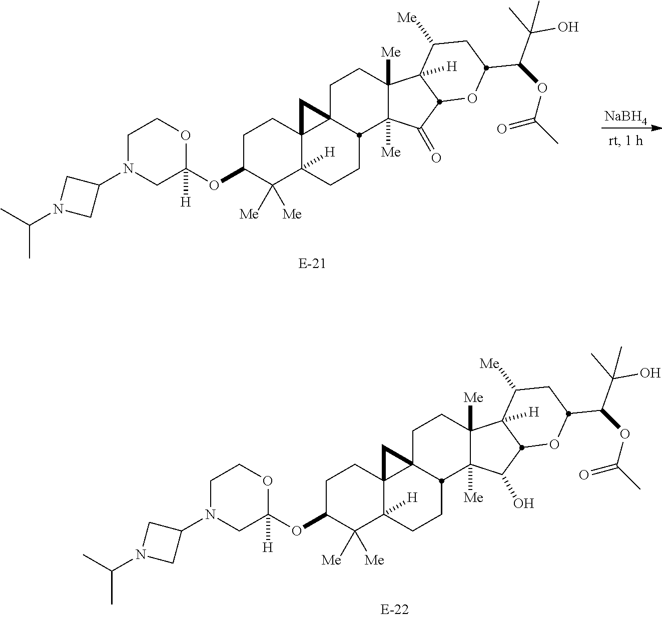

To the reaction mixture above was added NaBH₄ (2.0 equiv.) over 60 min at 20° C., and stirring was continued for another 70 min. Additional NaBH₄ (1.0 equiv.) was added over 15 min at 20° C., and stirring was continued for another 45 min. Additional NaBH₄ (0.91 equiv.) was added over 25 min at 20° C., and stirring was continued for 13 hrs.

### Step S-6

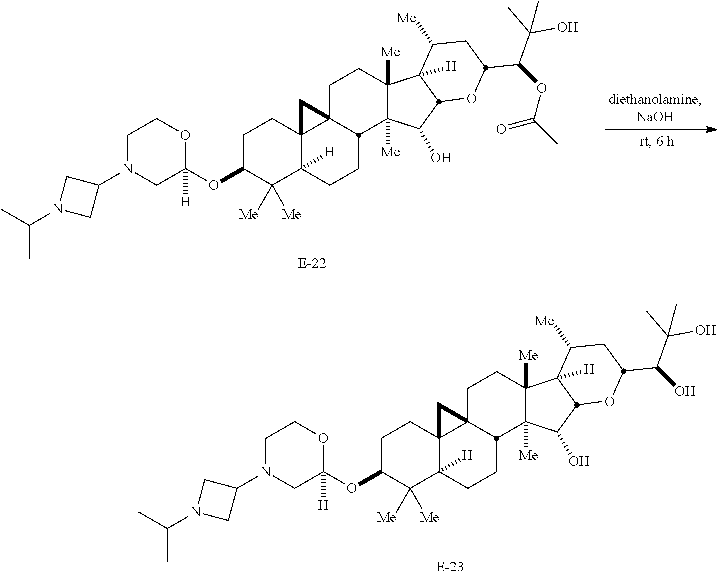

To the reaction mixture above was added diethanolamine (5.0 equiv.) over 5 min, and after stirring for 1 h, NaOH (30% aq. NaOH, 5.3 equiv.) was added over 10 min. Stirring was continued for 5 hrs at 21° C. Water and TBME were added to the reaction and extracted. The aqueous phase was re-extracted with TBME. The combined organic layers were washed with water, then washed again with the previously extracted aqueous phase. The combined organic phases were washed with 5.6% NaCl, and the resulting organic layer was concentrated under vac at 50° C. The residue was rinsed with DCM and evaporated under reduced pressure to provide crude E-23.

The crude product was purified by plug chromatography [DCM/(MeOH/25% aq. NH₃ 9:1) 95:5], then purified again using [DCM/(MeOH/25% aq. NH₃ 9:1) 90:10]. The fractions were concentrated under vac at 40-60° C./600-33 mbar to afford E-23 which was then dissolved in acetone and heated to 50° C. Water was added over 40 min at 50° C. The suspension was cooled to 18° C. over 3 hrs, and stirred. After another 11 hrs, the solid was filtered, and the filter cake was washed with acetone:water 2:1 and dried under vac to provide pure E-23.

### Step S-7

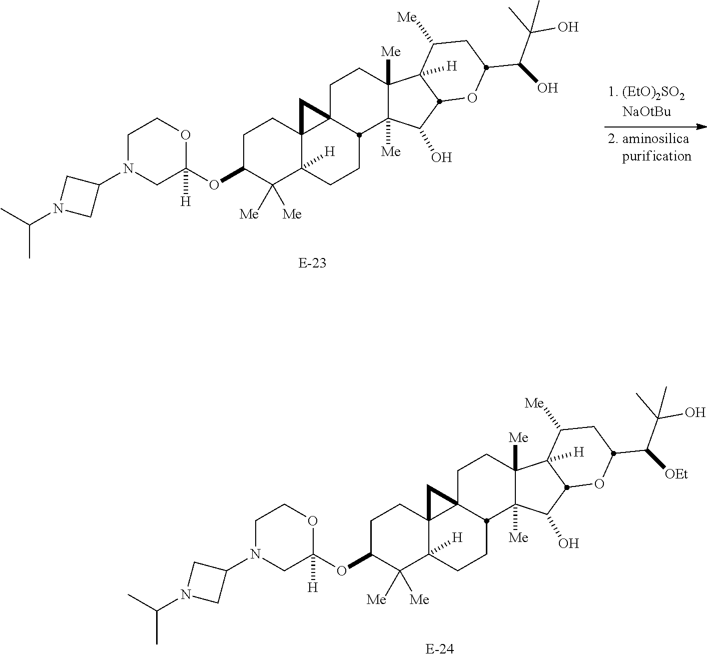

E-23 was azeotropically dried by concentrating from toluene under reduced pressure. This process was repeated twice, then the material was taken up in 5:1 toluene:DMF (0.2 M). The reaction was cooled to −1° C. and NaOtBu (5 equiv.) was added. The reaction was cooled to −20° C. and diethylsulphate (2 equiv.) was added over 15 min. The reaction was stirred for 3 h 15 min, and quenched with water over 15 min from −20 to 3° C. TBME was added and the mixture was warmed to 40° C. The aq. layer was re-extracted with toluene (3×) at 40° C., and the organic layer was washed with sat. brine (3×) at 40° C. The organic layer was concentrated under vac (44-60° C.) to provide crude E-24.

The crude E-24 was purified by plug chromatography [n-heptane/EtOAc 7:3], then [n-heptane/EtOAc 6:4], then [n-heptane/EtOAc 4:6], then EtOAc. The fractions were concentrated under vac at 60° C. to afford purified E-24. E-24 was then dissolved in TBME (10 volumes) and toluene (2.7 volumes) and heated to reflux. Water (0.05 volumes) was added, and the solution was seeded with crystals, followed by cooling to 10° C. over 120 min. The suspension was stirred for 12 hrs at 10° C., and filtered. The filter cake was washed with TBME (2 vol), and dried at 60° C. under vac (5 mbar) for 24 hrs, followed by drying at 70° C. under vac (5 mbar) for 24 hrs, followed by drying at 20° C. under vac (5 mbar) for 36 hrs to provide pure E-24 (79.9%).

### Example 7

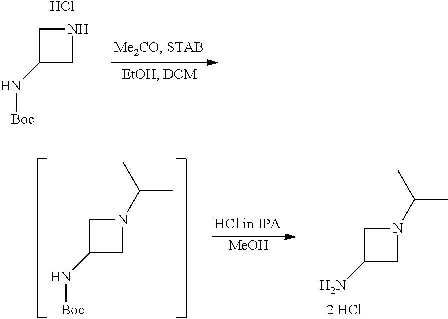

A flask was charged with 1-Boc-3-(amino)azetidine (1 equiv.), ethanol (0.4 M), DCM (1.6 M), acetone (3 equiv.), and NaBH(OAc)₃ (3 equiv.), and stirred for 18 hrs at 20° C. To the reaction was added acetone (0.5 equiv.) and NaBH(OAc)₃ (0.5 equiv.), and stirred for 2 hrs. The reaction mixture was extracted and distilled to remove some ethanol. To the organic layer was added 5M HCl in iPrOH (7 equiv.) at 50° C. TBME was added at 48° C. over 45 min, and cooled to 20° C. over 60 min. The reaction stirred for another 60 min, filtered, and the filter cake was washed with TBME. The organic layer was dried to provide 1-isopropylazetidin-3-amine dihydrochloride.

### Example 8

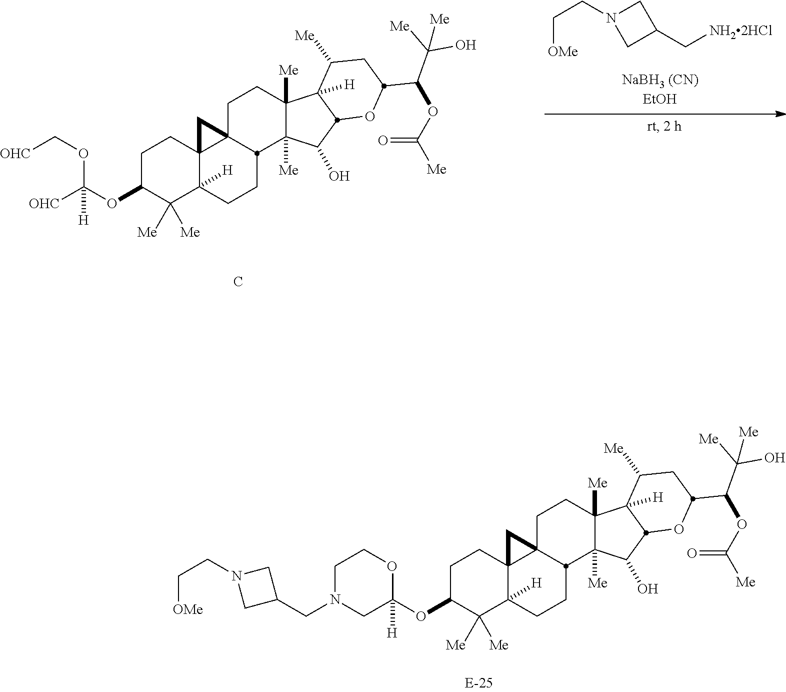

To the reaction mixture above was added 1-(2-methoxyethyl)-3-(methylamino)-azetidine dihydrochloride (1.2 equiv.) and ethanol (0.33 M). The mixture was stirred for 1 h at 20° C., and NaBH₃(CN) (1.2 equiv.) was added over 60 min, and stirring was continued for another 50 min. After 15 hrs, HPLC showed complete conversion of starting material, to provide E-25.

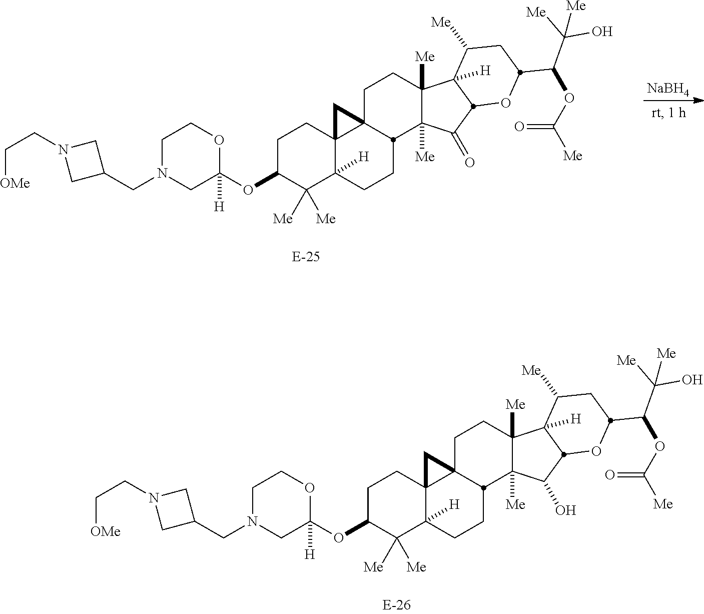

To the reaction mixture above was added NaBH₄ (2.0 equiv.) over 60 min at 20° C., and stirring was continued for another 70 min. Additional NaBH₄ (1.0 equiv.) was added over 15 min at 20° C., and stirring was continued for another 45 min. Additional NaBH₄ (0.91 equiv.) was added over 25 min at 20° C., and stirring was continued for 13 hrs.

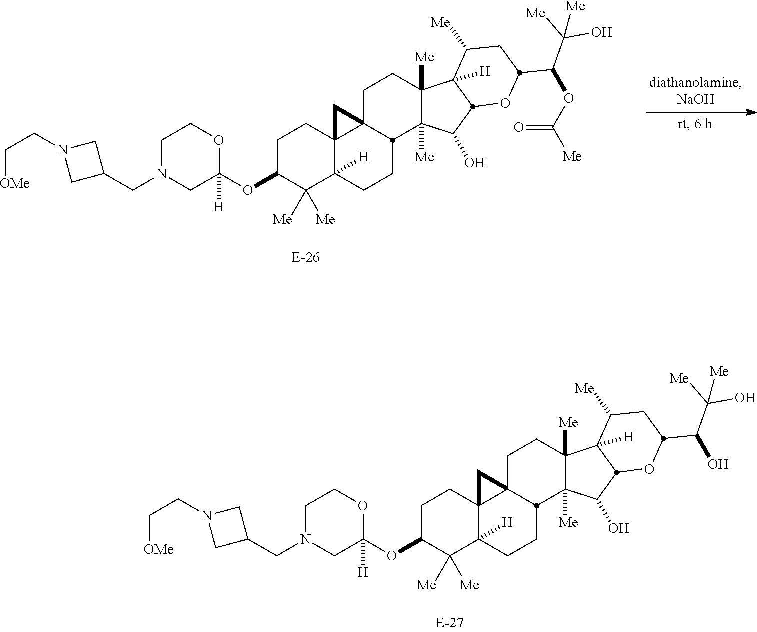

To the reaction mixture above was added diethanolamine (5.0 equiv.) over 5 min, and after stirring for 1 h, NaOH (30% aq. NaOH, 5.3 equiv.) was added over 10 min. Stirring was continued for 5 hrs at 21° C. Water and TBME were added to the reaction and extracted. The aqueous phase was re-extracted with TBME. The combined organic layers were washed with water, then washed again washed with again with aqueous phase 1. The combined organic phases were washed with 5.6% NaCl, and the resulting organic layer was concentrated under vac at 50° C. The residue was rinsed with DCM and evaporated under reduced pressure to provide crude E-27.

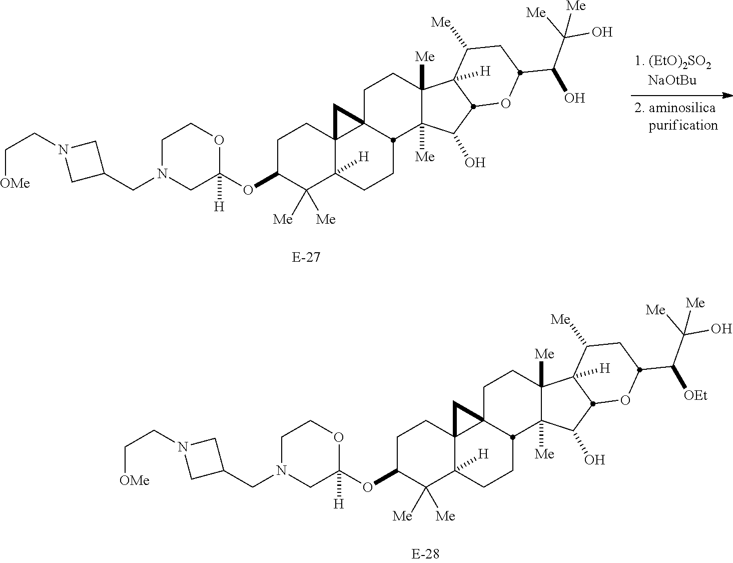

E-27 was azeotropically dried by concentrating from toluene under reduced pressure. This process was repeated twice, then the material was taken up in 5:1 toluene:DMF (0.2 M). The reaction was cooled to −1° C. and NaOtBu (5 equiv.) was added. The reaction was cooled to −20° C. and diethylsulphate (2 equiv.) was added over 15 min. The reaction was stirred for 3 h 15 min, and quenched with water over 15 min from −20 to 3° C. TBME was added and the mixture was warmed to 40° C. The aq. layer was re-extracted with toluene (3×) at 40° C., and the organic layer was washed with sat. brine (3×) at 40° C. The organic layer was concentrated under vac (44-60° C.) to provide crude E-28.

The crude E-28 was purified by plug chromatography [n-heptane:EtOAc 7:3], then [n-heptane/EtOAc 6:4], then [n-heptane/EtOAc 4:6], then EtOAc. The fractions were concentrated under vac at 60° C. to afford purified E-28. E-28 was then dissolved in TBME (10 volumes) and toluene (2.7 volumes) and heated to reflux. Water (0.05 volumes) was added, and the solution was seeded with crystals, followed by cooling to 10° C. over 120 min. The suspension was stirred for 12 hrs at 10° C., and filtered. The filter cake was washed with TBME (2 vol), and dried at 60° C. under vac (5 mbar) for 24 hrs, followed by drying at 70° C. under vac (5 mbar) for 24 hrs, followed by drying at 20° C. under vac (5 mbar) for 36 hrs to provide pure E-28 (79.9%).

### Example 9

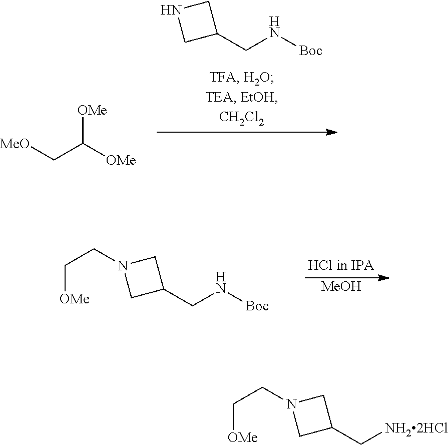

A flask was charged with methoxyacetaldehyde dimethylacetal (1.2 equiv.), trifluoroacetic acid (1.3 equiv.), and water (equal volume to TFA), and the mixture was stirred at 50° C. for 10 min. The reaction mixture was then removed from the heating bath and TEA (1.3 equiv.) was added followed by a solution of 3-(N-Boc-aminomethyl)-azetidine (1 equiv.) in EtOH and DCM, and then NaBH(OAc)₃ (3 equiv.). The reaction was stirred for 12 hrs at 20° C. The reaction mixture was extracted and distilled to remove some ethanol. To the organic layer was added 5M HCl in iPrOH (7 equiv.) at 50° C. TBME was added at 48° C. over 45 min, and cooled to 20° C. over 60 min. The reaction stirred for another 60 min, filtered, and the filter cake was washed with TBME. The organic layer was dried to provide 1-(2-methoxyethyl)-3-(methylamino)-azetidine dihydrochloride.

### Example 10

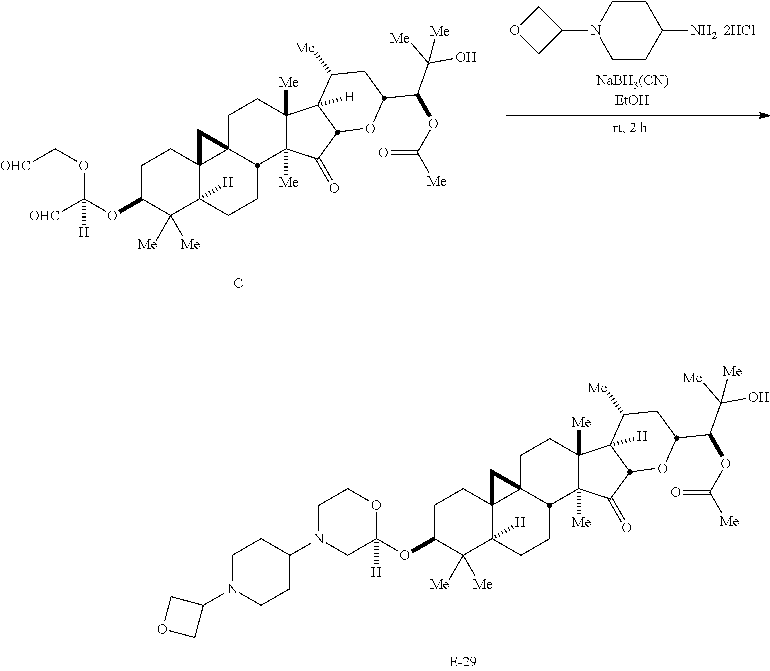

To the reaction mixture above was added 1-oxetane-4-amino-piperidine dihydrochloride (1.2 equiv.) and ethanol (0.33 M). The mixture was stirred for 1 h at 20° C., and NaBH₃(CN) (1.2 equiv.) was added over 60 min, and stirring was continued for another 50 min. After 15 hrs, HPLC showed complete conversion of starting material, to provide E-29.

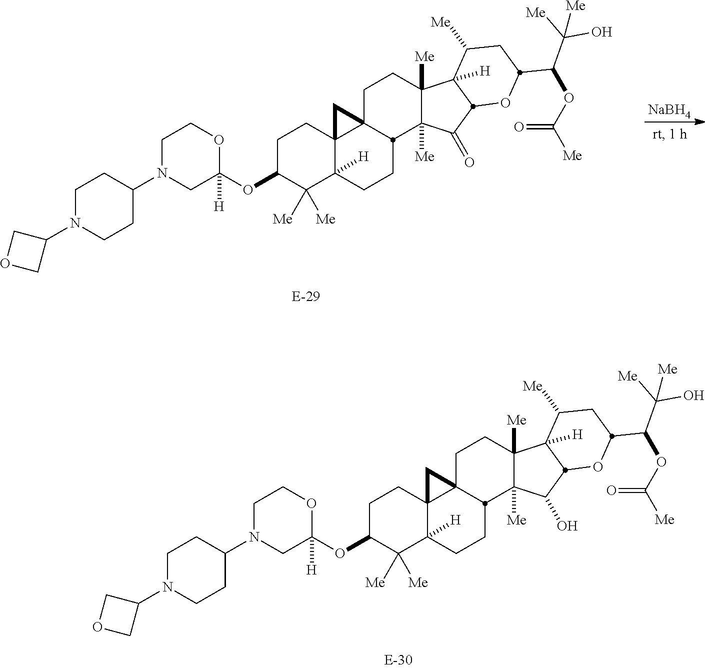

To the reaction mixture above was added NaBH₄ (2.0 equiv.) over 60 min at 20° C., and stirring was continued for another 70 min. Additional NaBH₄ (1.0 equiv.) was added over 15 min at 20° C., and stirring was continued for another 45 min. Additional NaBH₄ (0.91 equiv.) was added over 25 min at 20° C., and stirring was continued for 13 hrs.

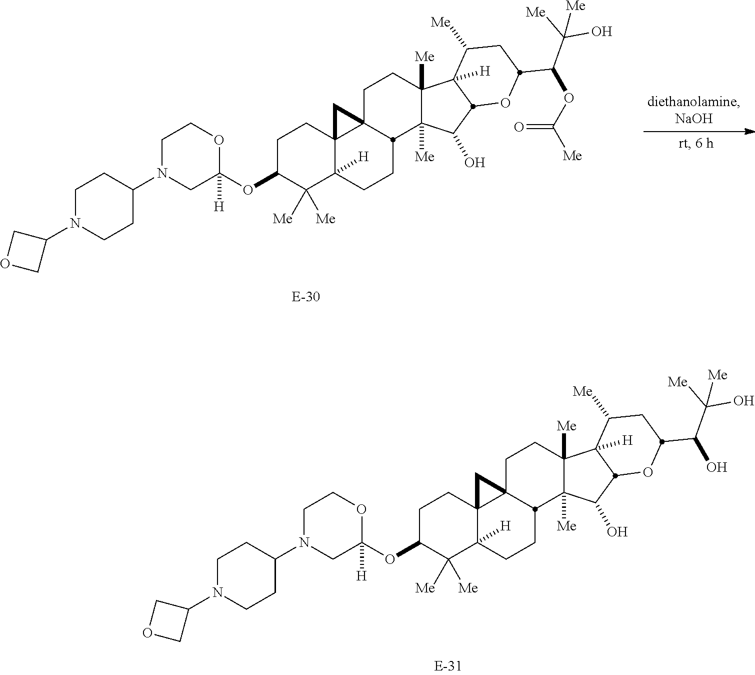

To the reaction mixture above was added diethanolamine (5.0 equiv.) over 5 min, and after stirring for 1 h, NaOH (30% aq. NaOH, 5.3 equiv.) was added over 10 min. Stirring was continued for 5 hrs at 21° C. Water and TBME were added to the reaction and extracted. The aqueous phase was re-extracted with TBME. The combined organic layers were washed with water, then washed again with the previously extracted aqueous phase. The combined organic phases were washed with 5.6% NaCl, and the resulting organic layer was concentrated under vac at 50° C. The residue was rinsed with DCM and evaporated under reduced pressure to provide crude E-31.

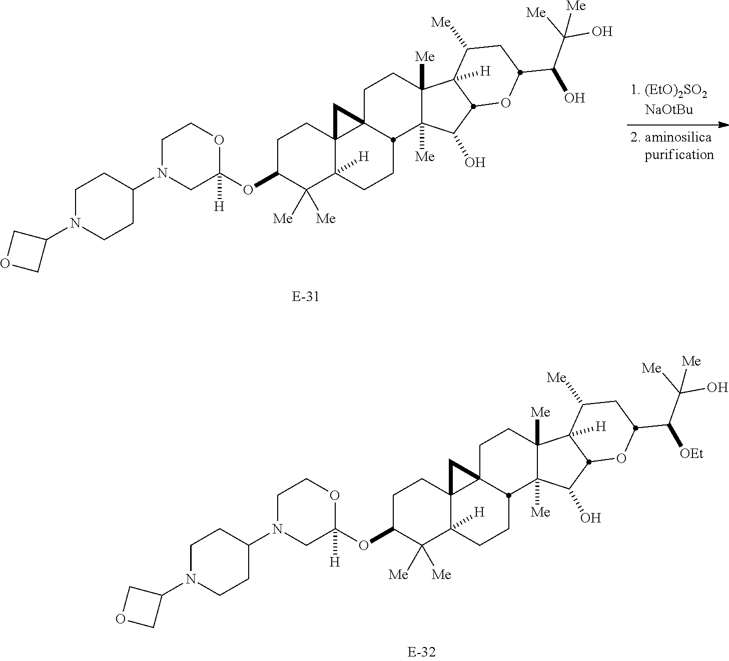

E-31 was azeotropically dried by concentrating from toluene under reduced pressure. This process was repeated twice, then the material was taken up in 5:1 toluene:DMF (0.2 M). The reaction was cooled to −1° C. and NaOtBu (5 equiv.) was added. The reaction was cooled to −20° C. and diethylsulphate (2 equiv.) was added over 15 min. The reaction was stirred for 3 h 15 min, and quenched with water over 15 min from −20 to 3° C. TBME was added and the mixture was warmed to 40° C. The aq. layer was re-extracted with toluene (3×) at 40° C., and the organic layer was washed with sat. brine (3×) at 40° C. The organic layer was concentrated under vac (44-60° C.) to provide crude E-32.

The crude E-32 was purified by plug chromatography [n-heptane/EtOAc 7:3], then [n-heptane/EtOAc 6:4], then [n-heptane/EtOAc 4:6], then EtOAc. The fractions were concentrated under vac at 60° C. to afford purified E-32. E-32 was then dissolved in TBME (10 volumes) and toluene (2.7 volumes) and heated to reflux. Water (0.05 volumes) was added, and the solution was seeded with crystals, followed by cooling to 10° C. over 120 min. The suspension was stirred for 12 hrs at 10° C., and filtered. The filter cake was washed with TBME (2 vol), and dried at 60° C. under vac (5 mbar) for 24 hrs, followed by drying at 70° C. under vac (5 mbar) for 24 hrs, followed by drying at 20° C. under vac (5 mbar) for 36 hrs to provide pure E-32 (79.9%).

### Example 11

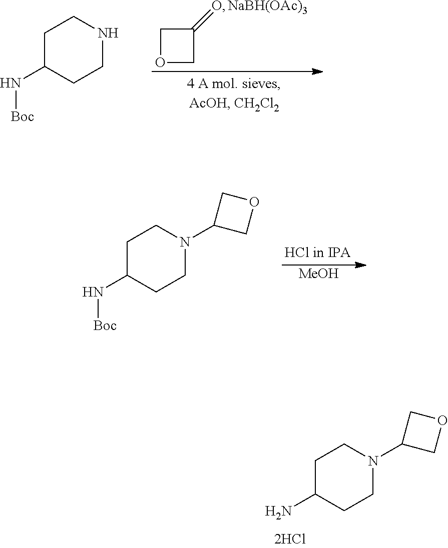 [SMILES: "MeOH" ; CC(C)(C)OC(=O)NC1CCNCC1 ; O=C1COC1 ; CC(C)(C)OC(=O)NC1CCN(C2COC2)CC1 ; NC1CCN(C2COC2)CC1 ; "NaBH(OAc)3"]

A solution of 4-N-Boc-amino-piperidine (1 equiv.) in DCM was treated with activated 4 Å molecular sieves, followed by AcOH (2 equiv.) and 3-oxetanone (2 equiv.). The reaction was stirred for 10 min, then NaBH(OAc)₃ (3.5 equiv.) was added. Stirring was continued at RT for 16 h, whereupon the reaction was filtered to remove sieves, and then partitioned between DCM and saturated aqueous NaHCO₃, and the layers were separated. Extracted with DCM, then concentrated under reduced pressure. To the residue was added 5M HCl in iPrOH (7 equiv.) at 50° C. TBME was added at 48° C. over 45 min, and cooled to 20° C. over 60 min. The reaction stirred for another 60 min, filtered, and the filter cake was washed with TBME. The organic layer was dried to provide 1-oxetane-4-amino-piperidine dihydrochloride.

Other compounds synthesized by the above examples include the compounds of Table 1.

Those skilled in the art will recognize, or be able to ascertain using no more than routine experimentation, many equivalents of the specific embodiments of the invention described herein. Such equivalents are intended with be encompassed by the following claims.

1. A method for preparing a compound of formula II: 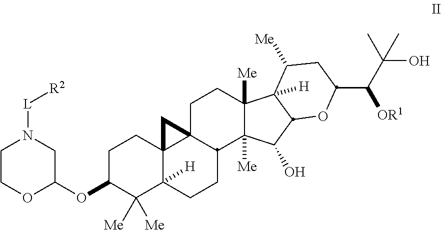
or a pharmaceutically acceptable salt thereof, wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R.
comprising the steps of:
(a) extracting from biomass a compound of formula A: 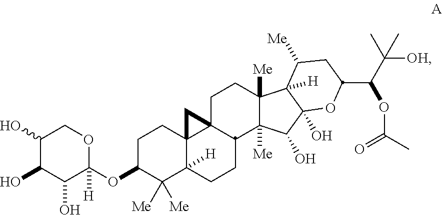
(b) treating said compound of formula A with a suitable acid to form a compound of formula B: 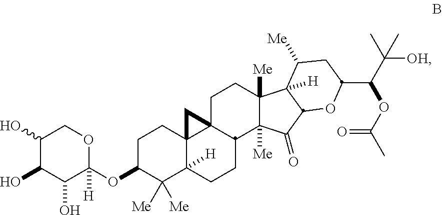
(c) treating said compound of formula B with a suitable oxidant to provide a compound of formula C: 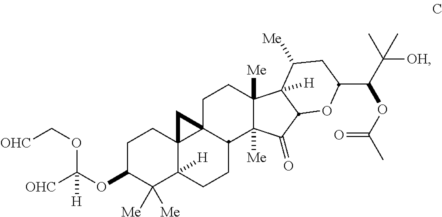
(d) treating said compound of formula C with a suitable amine, or salt thereof, in the presence of a base to provide a compound of formula D: 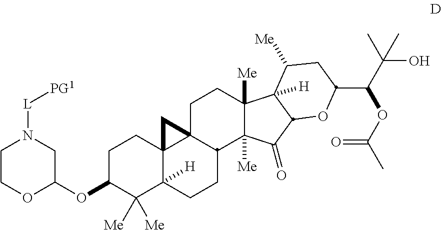
wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group;
(e) reducing the carbonyl component of said compound of formula D to form a compound of formula E: 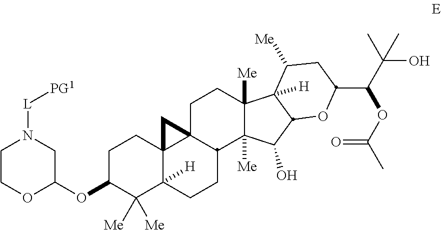
wherein:
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group;
(f) protecting said compound of formula E to form a compound of formula F:
wherein: 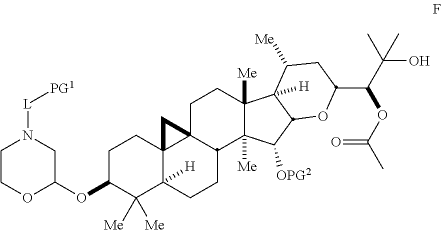
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(g) deacetylating said compound of formula F to form a compound of formula G:
wherein: 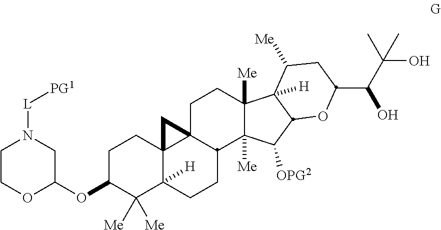
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(h) reacting said compound of formula G to form a compound of formula H: 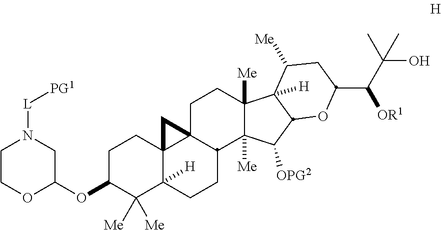
wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(i) deprotecting said compound of formula H to form a compound of formula J: 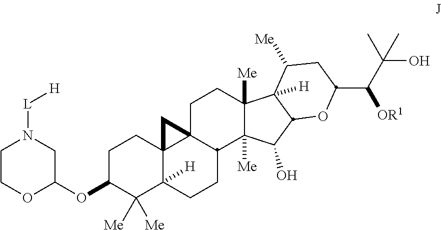
wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
(j) reacting said compound of formula J under suitable conditions to form a compound of formula II.

2. The method of claim 1, wherein L is a valence bond.

3. The method of claim 1, wherein L is an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-.

4. The method of claim 3, wherein each -Cy- is independently a bivalent optionally substituted saturated, partially unsaturated, or aromatic monocyclic or bicyclic ring selected from a 5-10 membered heteroarylene having 1-4 heteroatoms independently selected from oxygen, nitrogen, or sulfur, or a 3-10 membered heterocyclylene having 1-4 heteroatoms independently selected from oxygen, nitrogen, or sulfur.

5. The method of claim 3, wherein L is of either of the following formulae:

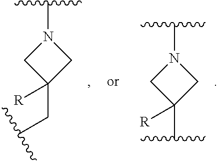

6. The method of claim 1, wherein PG¹ is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, or benzoyl.

7. The method of claim 1, wherein R² is R.

8. The method of claim 7, wherein R² is an optionally substituted C₁₋₆ heteroaliphatic group or optionally substituted C_{—-6} aliphatic group.

9. The method of claim 8, wherein R² is

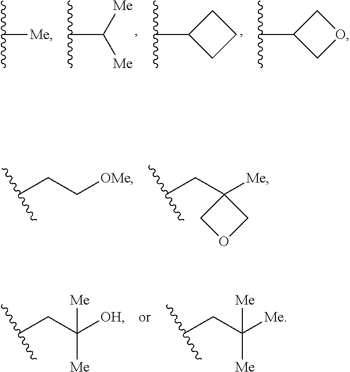

10. The method of claim 1, wherein step (h) is an alkylation reaction.

11. The method of claim 1, wherein the deprotection of step (i) occurs in a single step.

12. The method of claim 1, wherein step (j) is an N-alkylation reaction.

13. A method for preparing a compound of formula II: 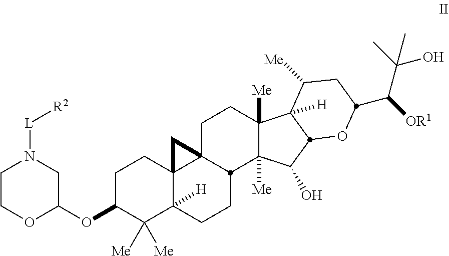
or a pharmaceutically acceptable salt thereof, wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
R² is halogen or R.
comprising the steps of:
(a) extracting from biomass a compound of formula A: 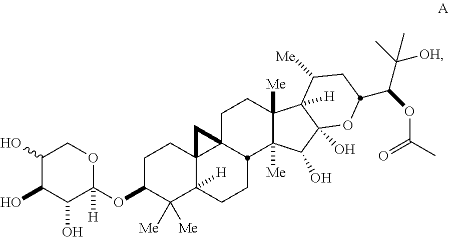
(b) treating said compound of formula A with a suitable acid to form a compound of formula B: 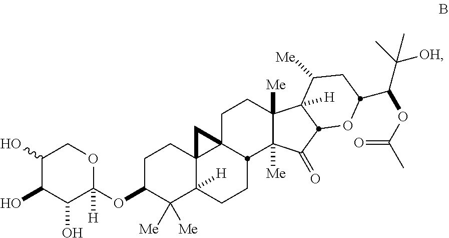
(c) treating said compound of formula B with a suitable oxidant to provide a compound of formula C: 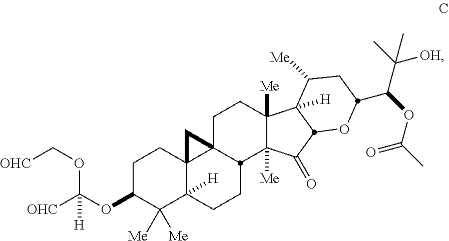
(d) treating said compound of formula C with a suitable amine, or salt thereof, in the presence of a base to provide a compound of formula D-1: 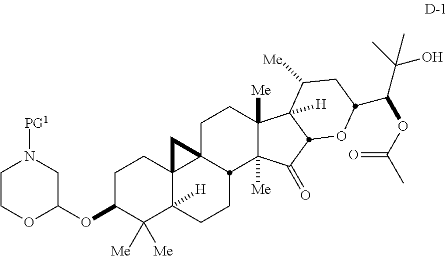
wherein:
PG¹ is a suitable amino protecting group;
(e) reducing the carbonyl component of said compound of formula D-1 to form a compound of formula E-1: 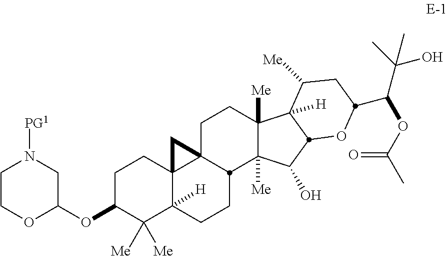
wherein:
PG¹ is a suitable amino protecting group;
(f) protecting said compound of formula E-1 to form a compound of formula F-1: 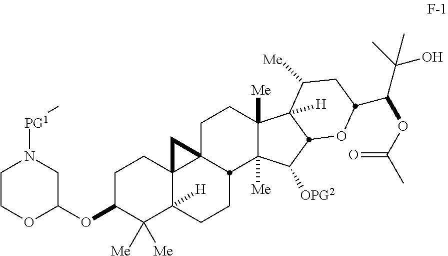
wherein:
PG¹ is a suitable amino protecting group; and
PG² is a suitable oxygen protecting group;
(g) deacetylating said compound of formula F-1 to form a compound of formula G-1: 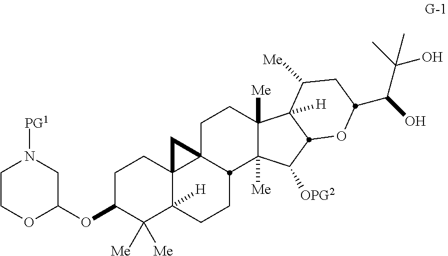
wherein:
PG¹ is a suitable amino protecting group; and
PG² is a suitable oxygen protecting group;
(h) reacting said compound of formula G-1 to form a compound of formula H-1: 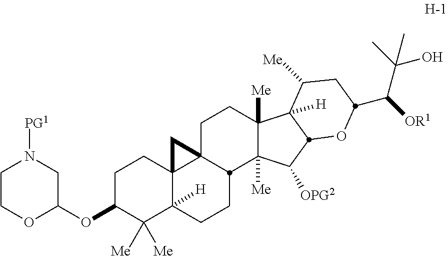
wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
PG¹ is a suitable amino protecting group; and
PG² is a suitable oxygen protecting group;
(i) deprotecting said compound of formula H-1 to form a compound of formula I: 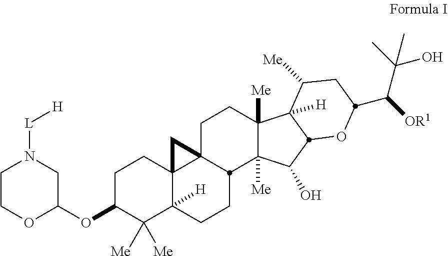
wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
(j) reacting said compound of formula I under suitable conditions to form a compound of formula II.

14. The method of claim 13, wherein L is a valence bond.

15. The method of claim 13, wherein L is an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-.

16. The method of claim 15, wherein each -Cy- is independently a bivalent optionally substituted saturated, partially unsaturated, or aromatic monocyclic or bicyclic ring selected from a 5-10 membered heteroarylene having 1-4 heteroatoms independently selected from oxygen, nitrogen, or sulfur, or a 3-10 membered heterocyclylene having 1-4 heteroatoms independently selected from oxygen, nitrogen, or sulfur.

17. The method of claim 16, wherein L is of either of the following formulae:

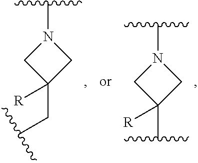

18. The method of claim 13, wherein PG¹ is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, or benzoyl.

19. The method of claim 13, wherein the deprotection at step (i) comprises a first step of deprotecting amino protecting group PG¹ and a second step of deprotecting oxygen protecting group PG².

20. The method of claim 13, wherein the formation of a compound of formula II at step (j) from a compound of formula I comprises steps of:
(i) reacting a compound of formula I under suitable conditions to provide a compound of formula K: 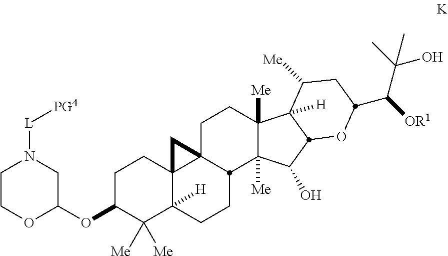
wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
and PG⁴ is an amino protecting group;
(ii) deprotecting a compound of formula K to provide a compound of formula J: 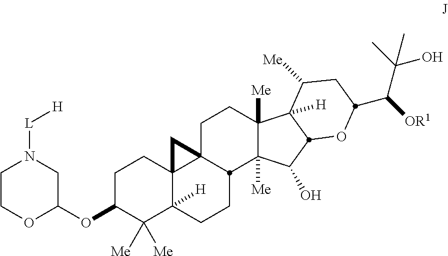
wherein:
R¹ is independently R, S(O)R, SO₂R, C(O)R, CO₂R, or C(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;
L is a valence bond or an optionally substituted C₁₋₁₀ alkylene chain wherein one, two, or three methylene units of L are optionally and independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or -Cy-, wherein:
each -Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur; and
each R is independently deuterium, hydrogen, an optionally substituted C₁₋₆ aliphatic group, an optionally substituted C₁₋₆ heteroaliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur; and
(iii) reacting a compound of formula J to provide a compound of formula II.

21-47. (canceled)
